SEQUENCE LISTING (46 sequences)

SEQ ID NO: 1 - DNA, 20 bp, Artificial sequence
  Synthetic sequence
gagactgagctggggtagct

SEQ ID NO: 2 - DNA, 18 bp, Artificial sequence
  Synthetic sequence
ccctaaccctaaccctaa

SEQ ID NO: 3 - DNA, 18 bp, Artificial sequence
  Synthetic sequence
gggattgggattgggatt

SEQ ID NO: 4 - DNA, 2166 bp, homo sapiens
aaaagaccacgtggggacgcggtggggcgggtctggcgggggcggggcacctctgtgcagggttcccagtcaccgcgacgctcctcgggaagccatagggcgcctcccagcccgtctccccgctccagtttagaacctaattcccaattcccggaccgggcccagccctgggctcttactgtccgcttttgctgggacctgttccacaaatgggcgtcttctgccttgggccgtgggggttgggccggaagctgcggacgcctgggaaggggccgctgcagctcttgagccgcctctgcggggaccacttgcaggccatcccagccaagaaggccccggctgggcaggaggagcctgggacgccgccctcctcgccgctgagtgccgagcagttggaccggatccagaggaacaaggccgcggccctgctcagactcgcggcccgcaacgtgcccgtgggctttggagagagctggaagaagcacctcagcggggagttcgggaaaccgtattttatcaagctaatgggatttgttgcagaagaaagaaagcattacactgtttatccacccccacaccaagtcttcacctggacccagatgtgtgacataaaagatgtgaaggttgtcatcctgggacaggatccatatcatggacctaatcaagctcacgggctctgctttagtgttcaaaggcctgttccgcctccgcccagtttggagaacatttataaagagttgtctacagacatagaggattttgttcatcctggccatggagatttatctgggtgggccaagcaaggtgttctccttctcaacgctgtcctcacggttcgtgcccatcaagccaactctcataaggagcgaggctgggagcagttcactgatgcagttgtgtcctggctaaatcagaactcgaatggccttgttttcttgctctggggctcttatgctcagaagaagggcagtgccattgataggaagcggcaccatgtactacagacggctcatccctcccctttgtcagtgtatagagggttctttggatgtagacacttttcaaagaccaatgagctgctgcagaagtctggcaagaagcccattgactggaaggagctgtgatcatcagctgaggggtggcctttgagaagctgctgttaacgtatttgccagttacgaagttccactgaaaattttcctattaattcttaagtactctgcataagggggaaaagcttccagaaagcagccatgaaccaggctgtccaggaatggcagctgtatccaaccacaaacaacaaaggctaccctttgaccaaatgtctttctctgcaacatggcttcggcctaaaatatgcagaagacagatgaggtcaaatactcagttggctctctttatctcccttgcctttatggtgaaacaggggagatgtgcacctttcaggcacagccctagtttggcgcctgctgctccttggttttgcctggttagactttcagtgacagatgttggggtgtttttgcttagaaaggtccccttgtctcagccttgcagggcaggcatgccagtctctgccagttccactgcccccttgatctttgaaggagtcctcaggcccctcgcagcataaggatgttttgcaactttccagaatctggcccagaaattagggctcaatttcctgattgtagtagaggttaagattgctgtgagctttatcagataagagaccgagagaagtaagctgggtcttgttattccttgggtgttggtggaataagcagtggaatttgaacaaggaagaggagaaaagggaattttgtctttatggggtggggtgattttctcctagggttatgtccagttggggtttttaaggcagcacagactgccaagtactgttttttttaaccgactgaaatcactttgggatattttttcctgcaacactggaaagttttagttttttaagaagtactcatgcagatatatatatatatatttttcccagtcctttttttaagagacggtctttattgggtctgcacctccatccttgatcttgttagcaatgctgtttttgctgttagtcgggttagagttggctctacgcgaggtttgttaataaaagtttgttaaaagtttaaaaaaaaaaaaaaaaaa

SEQ ID NO: 5 - protein, 304 aa, homo sapiens
MGVFCLGPWGLGRKLRTPGKGPLQLLSRLCGDHLQAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 6 - DNA, 2070 bp, homo sapiens
gccaattgctgaccgccacagccacagccagggctagcctcgccggttcccgggtggcgcgcgttcgctgcctcctcagctccaggatgatcggccagaagacgctctactcctttttctcccccagccccgccaggaagcgacacgcccccagccccgagccggccgtccaggggaccggcgtggctggggtgcctgaggaaagcggagatgcggcggccatcccagccaagaaggccccggctgggcaggaggagcctgggacgccgccctcctcgccgctgagtgccgagcagttggaccggatccagaggaacaaggccgcggccctgctcagactcgcggcccgcaacgtgcccgtgggctttggagagagctggaagaagcacctcagcggggagttcgggaaaccgtattttatcaagctaatgggatttgttgcagaagaaagaaagcattacactgtttatccacccccacaccaagtcttcacctggacccagatgtgtgacataaaagatgtgaaggttgtcatcctgggacaggatccatatcatggacctaatcaagctcacgggctctgctttagtgttcaaaggcctgttccgcctccgcccagtttggagaacatttataaagagttgtctacagacatagaggattttgttcatcctggccatggagatttatctgggtgggccaagcaaggtgttctccttctcaacgctgtcctcacggttcgtgcccatcaagccaactctcataaggagcgaggctgggagcagttcactgatgcagttgtgtcctggctaaatcagaactcgaatggccttgttttcttgctctggggctcttatgctcagaagaagggcagtgccattgataggaagcggcaccatgtactacagacggctcatccctcccctttgtcagtgtatagagggttctttggatgtagacacttttcaaagaccaatgagctgctgcagaagtctggcaagaagcccattgactggaaggagctgtgatcatcagctgaggggtggcctttgagaagctgctgttaacgtatttgccagttacgaagttccactgaaaattttcctattaattcttaagtactctgcataagggggaaaagcttccagaaagcagccatgaaccaggctgtccaggaatggcagctgtatccaaccacaaacaacaaaggctaccctttgaccaaatgtctttctctgcaacatggcttcggcctaaaatatgcagaagacagatgaggtcaaatactcagttggctctctttatctcccttgcctttatggtgaaacaggggagatgtgcacctttcaggcacagccctagtttggcgcctgctgctccttggttttgcctggttagactttcagtgacagatgttggggtgtttttgcttagaaaggtccccttgtctcagccttgcagggcaggcatgccagtctctgccagttccactgcccccttgatctttgaaggagtcctcaggcccctcgcagcataaggatgttttgcaactttccagaatctggcccagaaattagggctcaatttcctgattgtagtagaggttaagattgctgtgagctttatcagataagagaccgagagaagtaagctgggtcttgttattccttgggtgttggtggaataagcagtggaatttgaacaaggaagaggagaaaagggaattttgtctttatggggtggggtgattttctcctagggttatgtccagttggggtttttaaggcagcacagactgccaagtactgttttttttaaccgactgaaatcactttgggatattttttcctgcaacactggaaagttttagttttttaagaagtactcatgcagatatatatatatatatttttcccagtcctttttttaagagacggtctttattgggtctgcacctccatccttgatcttgttagcaatgctgtttttgctgttagtcgggttagagttggctctacgcgaggtttgttaataaaagtttgttaaaagtttaaaaaaaaaaaaaaaaaa

SEQ ID NO: 7 - protein, 313 aa, homo sapiens
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 8 - protein, 316 aa, Artificial sequence
  Synthetic construct
  misc_feature (1)..(7): Xaa can be any naturally occurring amino acid or absent
  misc_feature (8)..(10): Xaa can be any naturally occurring amino acid
  misc_feature (12)..(14): Xaa can be any naturally occurring amino acid
  misc_feature (16)..(18): Xaa can be any naturally occurring amino acid or absent
  misc_feature (19)..(19): Xaa can be any naturally occurring amino acid
  misc_feature (22)..(24): Xaa can be any naturally occurring amino acid
  misc_feature (26)..(28): Xaa can be any naturally occurring amino acid
  misc_feature (30)..(44): Xaa can be any naturally occurring amino acid
  misc_feature (35)..(39): Xaa can be any naturally occurring amino acid or absent
  misc_feature (40)..(47): Xaa can be any naturally occurring amino acid
XXXXXXXXXXFXXXPXXXXRKXXXPXXXPXXXXXXXXXXXXXXXXXXAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 9 - DNA, 597 bp, homo sapiens
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 10 - protein, 198 aa, homo sapiens [sequence omitted]

SEQ ID NO: 11 - protein, 198 aa, homo sapiens [sequence omitted]

SEQ ID NO: 12 - DNA, 2794 bp, homo sapiens
agagaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

SEQ ID NO: 13 - DNA, 3226 bp, homo sapiens
aagctgattgggtgtggtcgccgtggccggacgccgctcgggggacgtgggaggggaggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtggaatttgaaaaaaaaaaaaaaaaaa

SEQ ID NO: 14 - protein, 934 aa, homo sapiens [sequence omitted]

SEQ ID NO: 15 - DNA, 3042 bp, homo sapiens
aagctgattgggtgtggtcgccgtggccggacgccgctcgggggacgtgggaggggaggcgggaaacagcttagtgggtgtggggtcgcgcattttcttcaaccaggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtggaatttgaaaaaaaaaaaaaaaaaa

SEQ ID NO: 16 - protein, 868 aa, homo sapiens
MGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 17 - DNA, 4472 bp, homo sapiens
ccgcagacgcctgggaactgcggccgcgggctcgcgctcctcgccaggccctgccgccgggctgccatccttgccctgccatgtctcgccggaagcctgcgtcgggcggcctcgctgcctccagctcagcccctgcgaggcaagcggttttgagccgattcttccagtctacgggaagcctgaaatccacctcctcctccacaggtgcagccgaccaggtggaccctggcgctgcagcggctgcagcggccgcagcggccgcagcgcccccagcgcccccagctcccgccttcccgccccagctgccgccgcacatagctacagaaattgacagaagaaagaagagaccattggaaaatgatgggcctgttaaaaagaaagtaaagaaagtccaacaaaaggaaggaggaagtgatctgggaatgtctggcaactctgagccaaagaaatgtctgaggaccaggaatgtttcaaagtctctggaaaaattgaaagaattctgctgcgattctgcccttcctcaaagtagagtccagacagaatctctgcaggagagatttgcagttctgccaaaatgtactgattttgatgatatcagtcttctacacgcaaagaatgcagtttcttctgaagattcgaaacgtcaaattaatcaaaaggacacaacactttttgatctcagtcagtttggatcatcaaatacaagtcatgaaaatttacagaaaactgcttccaaatcagctaacaaacggtccaaaagcatctatacgccgctagaattacaatacatagaaatgaagcagcagcacaaagatgcagttttgtgtgtggaatgtggatataagtatagattctttggggaagatgcagagattgcagcccgagagctcaatatttattgccatttagatcacaactttatgacagcaagtatacctactcacagactgtttgttcatgtacgccgcctggtggcaaaaggatataaggtgggagttgtgaagcaaactgaaactgcagcattaaaggccattggagacaacagaagttcactcttttcccggaaattgactgccctttatacaaaatctacacttattggagaagatgtgaatcccctaatcaagctggatgatgctgtaaatgttgatgagataatgactgatacttctaccagctatcttctgtgcatctctgaaaataaggaaaatgttagggacaaaaaaaagggcaacatttttattggcattgtgggagtgcagcctgccacaggcgaggttgtgtttgatagtttccaggactctgcttctcgttcagagctagaaacccggatgtcaagcctgcagccagtagagctgctgcttccttcggccttgtccgagcaaacagaggcgctcatccacagagccacatctgttagtgtgcaggatgacagaattcgagtcgaaaggatggataacatttattttgaatacagccatgctttccaggcagttacagagttttatgcaaaagatacagttgacatcaaaggttctcaaattatttctggcattgttaacttagagaagcctgtgatttgctctttggctgccatcataaaatacctcaaagaattcaacttggaaaagatgctctccaaacctgagaattttaaacagctatcaagtaaaatggaatttatgacaattaatggaacaacattaaggaatctggaaatcctacagaatcagactgatatgaaaaccaaaggaagtttgctgtgggttttagaccacactaaaacttcatttgggagacggaagttaaagaagtgggtgacccagccactccttaaattaagggaaataaatgcccggcttgatgctgtatcggaagttctccattcagaatctagtgtgtttggtcagatagaaaatcatctacgtaaattgcccgacatagagaggggactctgtagcatttatcacaaaaaatgttctacccaagagttcttcttgattgtcaaaactttatatcacctaaagtcagaatttcaagcaataatacctgctgttaattcccacattcagtcagacttgctccggaccgttattttagaaattcctgaactcctcagtccagtggagcattacttaaagatactcaatgaacaagctgccaaagttggggataaaactgaattatttaaagacctttctgacttccctttaataaaaaagaggaaggatgaaattcaaggtgttattgacgagatccgaatgcatttgcaagaaatacgaaaaatactaaaaaatccttctgcacaatatgtgacagtatcaggacaggagtttatgatagaaataaagaactctgctgtatcttgtataccaactgattgggtaaaggttggaagcacaaaagctgtgagccgctttcactctccttttattgtagaaaattacagacatctgaatcagctccgggagcagctagtccttgactgcagtgctgaatggcttgattttctagagaaattcagtgaacattatcactccttgtgtaaagcagtgcatcacctagcaactgttgactgcattttctccctggccaaggtcgctaagcaaggagattactgcagaccaactgtacaagaagaaagaaaaattgtaataaaaaatggaaggcaccctgtgattgatgtgttgctgggagaacaggatcaatatgtcccaaataatacagatttatcagaggactcagagagagtaatgataattaccggaccaaacatgggtggaaagagctcctacataaaacaagttgcattgattaccatcatggctcagattggctcctatgttcctgcagaagaagcgacaattgggattgtggatggcattttcacaaggatgggtgctgcagacaatatatataaaggacagagtacatttatggaagaactgactgacacagcagaaataatcagaaaagcaacatcacagtccttggttatcttggatgaactaggaagagggacgagcactcatgatggaattgccattgcctatgctacacttgagtatttcatcagagatgtgaaatccttaaccctgtttgtcacccattatccgccagtttgtgaactagaaaaaaattactcacaccaggtggggaattaccacatgggattcttggtcagtgaggatgaaagcaaactggatccaggcgcagcagaacaagtccctgattttgtcaccttcctttaccaaataactagaggaattgcagcaaggagttatggattaaatgtggctaaactagcagatgttcctggagaaattttgaagaaagcagctcacaagtcaaaagagctggaaggattaataaatacgaaaagaaagagactcaagtattttgcaaagttatggacgatgcataatgcacaagacctgcagaagtggacagaggagttcaacatggaagaaacacagacttctcttcttcattaaaatgaagactacatttgtgaacaaaaaatggagaattaaaaataccaactgtacaaaataactctccagtaacagcctatctttgtgtgacatgtgagcataaaattatgaccatggtatattcctattggaaacagagaggtttttctgaagacagtctttttcaagtttctgtcttcctaacttttctacgtataaacactcttgaatagacttccactttgtaattagaaaattttatggacagtaagtccagtaaagccttaagtggcagaatataattcccaagcttttggagggtgatataaaaatttacttgatatttttatttgtttcagttcagataattggcaactgggtgaatctggcaggaatctatccattgaactaaaataattttattatgcaaccagtttatccaccaagaacataagaattttttataagtagaaagaattggccaggcatggtggctcatgcctgtaatcccagcactttgggaggccaaggtaggcagatcacctgaggtcaggagttcaagaccagcctggccaacatggcaaaaccccatctttactaaaaatataaagtacatctctactaaaaatacgaaaaaattagctgggcatggtggcgcacacctgtagtcccagctactccggaggctgaggcaggagaatctcttgaacctgggaggcggaggttgcaatgagccgagatcacgtcactgcactccagcttgggcaacagagcaagactccatctcaaaaaaaaaaaaagaaaaaagaaaagaaatagaattatcaagcttttaaaaactagagcacagaaggaataaggtcatgaaatttaaaaggttaaatattgtcataggattaagcagtttaaagattgttggatgaaattatttgtcattcattcaagtaataaatatttaatgaatacttgctataaaaaaaaaaaaaaaaaaaaaaaaaa

SEQ ID NO: 18 - protein, 1137 aa, homo sapiens
MSRRKPASGGLAASSSAPARQAVLSRFFQSTGSLKSTSSSTGAADQVDPGAAAAAAAAAAAAPPAPPAPAFPPQLPPHIATEIDRRKKRPLENDGPVKKKVKKVQQKEGGSDLGMSGNSEPKKCLRTRNVSKSLEKLKEFCCDSALPQSRVQTESLQERFAVLPKCTDFDDISLLHAKNAVSSEDSKRQINQKDTTLFDLSQFGSSNTSHENLQKTASKSANKRSKSIYTPLELQYIEMKQQHKDAVLCVECGYKYRFFGEDAEIAARELNIYCHLDHNFMTASIPTHRLFVHVRRLVAKGYKVGVVKQTETAALKAIGDNRSSLFSRKLTALYTKSTLIGEDVNPLIKLDDAVNVDEIMTDTSTSYLLCISENKENVRDKKKGNIFIGIVGVQPATGEVVFDSFQDSASRSELETRMSSLQPVELLLPSALSEQTEALIHRATSVSVQDDRIRVERMDNIYFEYSHAFQAVTEFYAKDTVDIKGSQIISGIVNLEKPVICSLAAIIKYLKEFNLEKMLSKPENFKQLSSKMEFMTINGTTLRNLEILQNQTDMKTKGSLLWVLDHTKTSFGRRKLKKWVTQPLLKLREINARLDAVSEVLHSESSVFGQIENHLRKLPDIERGLCSIYHKKCSTQEFFLIVKTLYHLKSEFQAIIPAVNSHIQSDLLRTVILEIPELLSPVEHYLKILNEQAAKVGDKTELFKDLSDFPLIKKRKDEIQGVIDEIRMHLQEIRKILKNPSAQYVTVSGQEFMIEIKNSAVSCIPTDWVKVGSTKAVSRFHSPFIVENYRHLNQLREQLVLDCSAEWLDFLEKFSEHYHSLCKAVHHLATVDCIFSLAKVAKQGDYCRPTVQEERKIVIKNGRHPVIDVLLGEQDQYVPNNTDLSEDSERVMIITGPNMGGKSSYIKQVALITIMAQIGSYVPAEEATIGIVDGIFTRMGAADNIYKGQSTFMEELTDTAEIIRKATSQSLVILDELGRGTSTHDGIAIAYATLEYFIRDVKSLTLFVTHYPPVCELEKNYSHQVGNYHMGFLVSEDESKLDPGAAEQVPDFVTFLYQITRGIAARSYGLNVAKLADVPGEILKKAAHKSKELEGLINTKRKRLKYFAKLWTMHNAQDLQKWTEEFNMEETQTSLLH

SEQ ID NO: 19 - DNA, 4435 bp, homo sapiens
ggcgaggcgcctgttgattggccactggggcccgggttcctccggcggagcgcgcctccccccagatttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgctccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgacaaaggtggtaaattcagacaacattatgatctaataaactttattttttaaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - protein, 1360 aa, homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 21 - DNA, 2662 bp, homo sapiens
gaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacataa

SEQ ID NO: 22 - protein, 756 aa, homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 23 - DNA, 5156 bp, homo sapiens
agccaatgggagttcaggaggcggagcgcctgtgggagccctggagggaactttcccagtccccgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttccgatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctaaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaacctgctacttaaaaaaaatacacatcacacccatttaaaagtgatcttgagaaccttttcaaaccagatggagcattgcttgcaaattttttttctctatgtttgcatgcgctcgtgtgtgtgtgtccaggcaagaacacattttataaaaataagaacacttgggctgggcatggtggctcatgcctgtgatcgcagcactttgggaggccgaggccggcggatcacctgagatcagaagttcgagaccagcctgaccaacatggagaaaccctgcctctactaaaaatacaaaattagccaggtgtgctggcgcatgcctgtaatccccgctacccaggaggctgaggcaggagaatcgcttgaacccgggagacggaggttgcagtgaaccgagattgcgccactgcgctccagcctgggtgagatagaacaagactgtgtctcaaaaaacaaaacaaaacaaaacaaaaaaaaaaaaaccaaaccactttggaagttactcaggcctctgctctggctggacatagtttagtctataactttcaacccttaatgataattaaattcatctttgtttaatttcataaatttaaaagtagggtccttttcagttagtgattctcagccctgattcacattaaatttttaaacacgggggattctctgcccggctggaagaaaatgactggatgggacaggggtcactatttgaaacattcctctgtgcggccaaggtcgcaaaatgctgtcctcgcaggggaacaaaaagagtttgatttcccataatttgatgctgtgatttggtttcctcaggatgtgaactgtagaacattccagttactggccttgaatggttctgggaatataagaatccctgtctgtcttttcaaatagttttcatggaaccttgtcctgtttgaacttggctgaaaatggaagtaaagatgccctcttgggggcccagagatgacagatgtggctccccctgctgcccccaccccttctccagactgtgggcggctccccttcctgctttagaatccctcagatggaggaggcagtacagtagtcactgtgccatcgtgtctggcactgtgctggcgtggtctgcaggatcccacttatgaactctccagattgggagctgtggcaggataacagcccccaagacagctgtgtcctaatccccagaacctgtgaccacgctgcctcacgtggcagaagggactcggcaggtgtgattgagtgaaggatcttttttttttttttctttgagatgaagtttcgctcttgttgcccaggctggagttcaatagcatgatctcagctcactgcagcctctgcctcccaggttcaagtgattctcccacctcagcctcccgagtagctgggattacaggtgtccagaaccatactggctaatttttgtatttttagtagagacagggtttcaccatgttgaccaggctggtctcgaactcctgacctcaggtgatccgaccgcctcggcctcccaaagtgctgggattacaggtgtgagccatcatgcctggctgagttaaggatcttgcaacagagagattatcctggattgtctgggtgggcccagtccattgggtgagtccttcaaaggtggagacctttccctgctggccagagagaggctgtcttgctggttttggagatggaaggaggtaccactagtcaaggattgcaagcagtctctagaacagggattccaacactccggacacagaccagtagtggtccatggcctattaggaagtggggtgcacagcaggttaggggccggcaagccagcgaagcttcatctgtatttatagccactccccgtcgctggcgttaccacccgagctccgcctcctgtcacatcagcggtgggcattagattctcatagcagcacgagccctattgtgaactgcacacacgagggatgtaggttgcacgctccttatgagaatctgatgcctgatgatctgtcactgtctcccgtcacccccagatggggctgtctagttgcaggaaaacaagctcagggctcccactgagtctctgtgatggtgagttgtagaattatttaattatatgttacaatgtaataatagtagaaataaagtgcacaataaatgcaatgcacttgaatcgtcctgaaaccatccctccccgaccccaatccatggaaaaattgtgttccgcgaaaccggtctctggtgccaaaaaggttggggaccgcttctggaaaagctggaaaaggcaagaaaacgcattctctccctcagcctctggaaggaaccagcactgtgggactaatttacatactgtagggtaataaatttgtgttgcttcgaaccactaaaaaaaaa

SEQ ID NO: 24 - protein, 862 aa, homo sapiens
MERAESSSTEPAKAIKPIDRKSVHQICSGQVVLSLSTAVKELVENSLDAGATNIDLKLKDYGVDLIEVSDNGCGVEEENFEGLTLKHHTSKIQEFADLTQVETFGFRGEALSSLCALSDVTISTCHASAKVGTRLMFDHNGKIIQKTPYPRPRGTTVSVQQLFSTLPVRHKEFQRNIKKEYAKMVQVLHAYCIISAGIRVSCTNQLGQGKRQPVVCTGGSPSIKENIGSVFGQKQLQSLIPFVQLPPSDSVCEEYGLSCSDALHNLFYISGFISQCTHGVGRSSTDRQFFFINRRPCDPAKVCRLVNEVYHMYNRHQYPFVVLNISVDSECVDINVTPDKRQILLQEEKLLLAVLKTSLIGMFDSDVNKLNVSQQPLLDVEGNLIKMHAADLEKPMVEKQDQSPSLRTGEEKKDVSISRLREAFSLRHTTENKPHSPKTPEPRRSPLGQKRGMLSSSTSGAISDKGVLRPQKEAVSSSHGPSDPTDRAEVEKDSGHGSTSVDSEGFSIPDTGSHCSSEYAASSPGDRGSQEHVDSQEKAPKTDDSFSDVDCHSNQEDTGCKFRVLPQPTNLATPNTKRFKKEEILSSSDICQKLVNTQDMSASQVDVAVKINKKVVPLDFSMSSLAKRIKQLHHEAQQSEGEQNYRKFRAKICPGENQAAEDELRKEISKTMFAEMEIIGQFNLGFIITKLNEDIFIVDQHATDEKYNFEMLQQHTVLQGQRLIAPQTLNLTAVNEAVLIENLEIFRKNGFDFVIDENAPVTERAKLISLPTSKNWTFGPQDVDELIFMLSDSPGVMCRPSRVKQMFASRACRKSVMIGTALNTSEMKKLITHMGEMDHPWNCPHGRPTMRHIANLGVISQN

SEQ ID NO: 25 - DNA, 3208 bp, homo sapiens
gcctaaggaaacgtgtcgtctggaatgggcttgggggccacgcctgcacatctccgcgagacagagggataaagtgaagatggtgctgttattgttacctcgagtgccacatgcgacctctgagatatgtacacagtcattcttactatcgcactcagccattcttactacgctaaagaagaaataattattcgaggatatttgcctggcccagaagaaacttatgtaaatttcatgaactattatatccgttttcctcggagtgagagaaaactctttttagatatcatctgagaggtagttaatttggcaccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaattctgaaaagcttcctccttgtaagaaacccctgtccccagtcagagataacatccaactaactccagaagcggaagaggatatatttaacaaacctgaatgtggccgtgttcaaagagcaatattccagtaaatgcagactgctgcaaagcttttgcctgcaagagaatctgatcaatttgaagtccctgtttgggaatgaggcacttatcagcatgaagaattttttctcattctgtgccattttaaaaatagaatacattttgtatattaactttataattgggttgtggtttttttgctcagctttttatatttttataagaagctaaatagaagaataattgtatctctgacaggtttttggaggttttagtgttaattgggaaaatcctctggagtttataaaagtctactctaaatatttctgtaatgttgtcaagtagaaagatagtaaatggagaaactacaaaaaaaaaaaaaaaaaa

SEQ ID NO: 26 - protein, 803 aa, homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 27 - DNA, 3370 bp, homo sapiens
aaattgaaaggtcagcctttcgcgcgctgtgtaggcaagttacccgtgttctgcgttgccggccgtgggtgctctggccacagtgagttaggggcgtcggagcgggtttctccaaccgcaatcggctccgctcaaggggaggaggagagtcccttctcggaaggcctaaggaaacgtgtcgtctggaatgggcttgggggccacgcctgcacatctccgcgagacagagggataaagtgaagatggtgctgttattgttacctcgagtgccacatgcgacctctgagatatgtacacagtcattcttactatcgcactcagccattcttactacgctaaagaagaaataattattcgaggatatttgcctggcccagaagaaacttatgtaaatttcatgaactattatatccgttttcctcggagtgagagaaaactctttttagatatcatctgagaggtagttaatttggcaccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaaatgcagactgctgcaaagcttttgcctgcaagagaatctgatcaatttgaagtccctgtttgggaatgaggcacttatcagcatgaagaattttttctcattctgtgccattttaaaaatagaatacattttgtatattaactttataattgggttgtggtttttttgctcagctttttatatttttataagaagctaaatagaagaataattgtatctctgacaggtttttggaggttttagtgttaattgggaaaatcctctggagtttataaaagtctactctaaatatttctgtaatgttgtcaagtagaaagatagtaaatggagaaactacaaaaaaaaaaaaaaaaaa

SEQ ID NO: 28 - protein, 845 aa, homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 29 - protein, 84 aa, bacillus subtilis
MTNLSDIIEKETGKQLVIQESILMLPEEVEEVIGNKPESDILVHTAYDESTDENVMLLTSDAPEYKPWALVIQDSNGENKIKML

SEQ ID NO: 30 - DNA, 21 bp, Artificial sequence
  Synthetic sequence
gtgccacctcctgctcactgg

SEQ ID NO: 31 - DNA, 21 bp, Artificial sequence
  Synthetic sequence
ttcatgtagcccttcccaggc

SEQ ID NO: 32 - DNA, 21 bp, Artificial sequence
  Synthetic sequence
actccactcacggcaaattca

SEQ ID NO: 33 - DNA, 20 bp, Artificial sequence
  Synthetic sequence
gcctcaccccatttgatgtt

SEQ ID NO: 34 - DNA, 21 bp, mus musculus
gcgagatgcatttcgtatgtt

SEQ ID NO: 35 - DNA, 21 bp, mus musculus
gaagtcgatgacttgcgagat

SEQ ID NO: 36 - DNA, 21 bp, homo sapiens
ctttggttatcttcgcaataa

SEQ ID NO: 37 - DNA, 21 bp, homo sapiens
gccatcatgaccttcaaagat

SEQ ID NO: 38 - DNA, 21 bp, mus musculus
ccatttattgagaccagacat

SEQ ID NO: 39 - DNA, 21 bp, homo sapiens
gccttgctgaataagtgtaaa

SEQ ID NO: 40 - DNA, 21 bp, homo sapiens
cctggcaatctctctcagttt

SEQ ID NO: 41 - DNA, 21 bp, mus musculus
cgagatcatttcacggataaa

SEQ ID NO: 42 - DNA, 21 bp, mus musculus
gctcacttagacgccattgtt

SEQ ID NO: 43 - DNA, 22 bp, Artificial sequence
  Synthetic sequence
gcaagctgaccctgaagttcat

SEQ ID NO: 44 - DNA, 20 bp, Artificial sequence
  Synthetic sequence
ctctgactcgaccccatcga

SEQ ID NO: 45 - DNA, 42 bp, Artificial sequence
  Synthetic sequence
  misc_feature (16)..(16): n is u
attattattattccgnggatttatttatttatttatttattt

SEQ ID NO: 46 - DNA, 42 bp, Artificial sequence
  Synthetic sequence
aaataaataaataaataaataaatccgcggaataataataat

(19) United States
(12) Patent Application Publication
(10) Pub. No.: US 2018/0221438 A1
(43) Pub. Date: Aug. 9, 2018

(54) MODULATING URACIL-DNA GLYCOSYLASE AND USES THEREOF

(71) Applicants: Adaerata Limited Partnership, Montreal (CA); University of Miami, Miami, FL (US)

(72) Inventors: Javier M. DI NOIA, Montreal (CA); Ramiro E. VERDUN, Miami Beach, FL (US); Stephen Patrick METHOT, Montreal (CA)

(21) Appl. No.: 15/569,916

(22) PCT Filed: Apr. 27, 2016
(86) PCT No.: PCT/CA2016/050486

§ 371 Date: Oct. 27, 2017

Related U.S. Application Data
(60) Provisional application No. 62/153,072, filed on Apr. 27, 2015.

(51) Int. Cl. A61K 38/16; A61K 35/76; A61K 38/15; A61P 35/02; C07K 14/32; C07K 11/02; A61K 31/513; G01N 33/574

(52) U.S. Cl. CPC A61K 38/16; A61K 35/76; A61K 38/15; A61P 35/02; A61K 2300/00; C07K 11/02; A61K 31/513; G01N 33/57426; G01N 2333/924; C07K 14/32

(57) ABSTRACT

The present invention concerns a method for the prevention and/or treatment of an activation-induced deaminase (AID)-associated disease in a subject in need thereof, said method comprising administering an effective amount of an uracil-DNA glycosylase (UNG) inhibitor, or a composition comprising the inhibitor, and a pharmaceutically acceptable carrier, to a subject having pathogenic cells expressing AID, uracil-DNA glycosylase (UNG) and mismatch repair pathway (MMR). Also provided are kits comprising an UNG inhibitor, methods of stratifying a subject having an AID-associated disease, uses and compositions for use of the UNG inhibitor.

### CROSS REFERENCE TO RELATED APPLICATIONS

This application is a PCT application Ser. No. PCT/CA2016/* filed on Apr. 27, 2016 and published in English under PCT Article 21(2), which itself claims benefit of U.S. provisional application Ser. No. 62/153,072, filed on Apr. 27, 2015. All documents above are incorporated herein in their entirety by reference.

### STATEMENT REGARDING FEDERALLY SPONSORED RESEARCH OR DEVELOPMENT

N.A.

### FIELD OF THE INVENTION

The present invention relates to modulating uracil-DNA glycosylase (UNG) and uses thereof. More particularly, it relates to modulating UNG in a subject having activation-induced deaminase (AID)-expressing pathogenic (e.g., neoplastic) cells.

### REFERENCE TO SEQUENCE LISTING

Pursuant to 37 C.F.R. 1.821(c), a sequence listing is submitted herewith as an ASCII compliant text file named 12810578_ST25.txt, that was created on Apr. 27, 2016 and having a size of 129 kilobytes. The content of the aforementioned file named 12810578_ST25.txt is hereby incorporated by reference in its entirety.

### BACKGROUND OF THE INVENTION

The germinal center (GC) reaction of antigen-activated B-cells is the hallmark of antibody-mediated immune responses to T-cell-dependent antigens ¹. During the GC reaction, B-cells undergo not only clonal expansion but also somatic hypermutation and class switching of their antibodies. Antibody class switch recombination (CSR) is a chromosomal rearrangement within the immunoglobulin (Ig) locus that involves the replacement of the IgM constant region (Cμ) with a downstream CH exon (δ, γ, or α), thereby switching the antibody isotype expressed without changing the receptor specificity². CSR involves large repetitive switch (S)-region sequences located upstream of each CH exon. These S-regions are G-rich tandem repetitive DNA up to 12 kb in length and are located up to ˜200 kb apart. CSR is initiated by activation-induced deaminase (AID), which converts the cytosine (C) base of deoxycytidine into uracil (U) over the S-regions. AID is active in single-stranded DNA (ssDNA) and preferentially deaminates dC present in the 5′-WRCY-3′ (W=A/T, R=A/G, Y=C/T) motif, which are present at high density in the S-regions of the Ig locus ².

Although the molecular mechanism used by AID to associate with the Ig locus is still under study, it is accepted that transcription-linked events play an essential role in the recruitment of AID most likely by exposing ssDNA ². The link between AID localization and transcription is also supported by reports showing that during CSR AID interacts with RPII ³, the transcription factor SPT5 ⁴, and the RNA exosome ⁵, among others. AID is expressed at high levels in germinal center B-cells with a very stringent temporal and spatial regulation ^{6,7}. However GC-derived B-cell lymphomas such as Diffuse Large B-cell Lymphoma (DLBCL) or Burkitt's lymphoma, among others, can also show expression of AID ^{8,9}.

At least two different DNA repair pathways can recognize uracil moieties in the S-regions. The most prominent pathway for this is base excision repair (BER) initiated by the nuclear isoform of the uracil-DNA glycosylase UNG (UNG1 refers to the mitochondrial and UNG2 to the nuclear isoform, the term “UNG” is used hereafter to refer to the nuclear isoform), which excises U to create an abasic site. Processing of this abasic site produces single-stranded breaks (SSBs) that act as intermediates for generating DNA double-stranded breaks (DSBs) needed for CSR. The other pathway is mismatch repair (MMR), which uses the heterodimeric recognition complex MSH2/MSH6 to detect G:U mismatches and initiates the formation of the DSBs needed for CSR. These DSBs induce a DNA damage response that involves proteins such as the PI3-like protein kinases, Ataxia Telangiectasia Mutated (ATM), DNA-dependent protein kinase catalytic subunit (DNAPkc), the MRE11-RAD50-NBS1 (MRN) complex, the heterodimer Ku70/80, and p53 Binding Protein 1 (53BP1)^{2,10}.

These DNA damage response proteins are essential for end processing of DSBs and for enabling non-homologous end-joining pathways activity. These pathways induce the recombination step where the intervening region between recombining segments is circularized and lost, and the chromosome rejoined ^{11,12}. An unavoidable side effect of using AID for shaping an efficient immune response is that it also mutates other regions of the genome, thereby creating a predisposition for B-cell lymphomas and leukemia.

More particularly, AID produces off-target deaminations and DNA damage, which unless faithfully repaired can be oncogenic. UNG and MSH2/MSH6 can modulate the mutagenic capacity of AID by either initiating error free excision repair (BER) and mismatch DNA repair (MMR), respectively, or by triggering mutagenic repair.

Indeed, AID mutates proto-oncogenes like Bcl6, Pim1, Pax5, CD95 and c-Myc in human B-cell lymphomas and normal B-cells ¹³⁻²⁰. Thus, any of the intermediate DNA lesions formed during antibody diversification (U, abasic sites, DSBs) can be tumorigenic if they happen off-target and are not properly resolved. More dramatically, DSBs can lead to deleterious chromosomal translocations, which are a hallmark of B-cell malignancies. In addition, these lesions can also be cytotoxic if not repaired, and it is presumably through this effect that AID controls the size of the GC ²¹. The full extent of off-target AID activity and the repair mechanisms that control it are not yet known

Neoplastic disease

The transformation of a normal cell into a malignant cell results, among other things, in the uncontrolled proliferation of the progeny cells, which exhibit immature, undifferentiated morphology, exaggerated survival and pro-angiogenic properties. Once a tumor has formed, cancer cells can leave the original tumor site and migrate to other parts of the body via the bloodstream and/or the lymphatic system by a process called metastasis. In this way, the disease may spread from one organ or part to another non-contiguous organ or part.

The increased number of cancer cases reported around the world is a major concern. Currently there are only a handful of treatments available for specific types of cancer and these treatments provide only limited efficacy and are often associated with toxicity. In addition, one of the biggest concerns of all cancer treatments is the development of chemotherapy resistance.

All steps of cancer progression as well as the development of drug resistance arise as a result of the acquisition of a series of fixed DNA sequence abnormalities, mutations, many of which ultimately confer a growth advantage upon the cells in which they have occurred. Some mutations lead, for example, to the overexpression or constitutive activation of oncogenes not normally expressed by normal mature cells.

Tumor profiling

Although the understanding of the molecular pathogenesis of cancer has advanced in the last two decades, risk assessment continues to be solely based on a few clinical parameters. Many studies conducted in recent years support the concept that the prognostic assessment of cancer should routinely include the investigation of molecular biomarkers. Also, because side effects of many treatments are severe, there is a need for targeted therapy. In cancer therapy, the quest for better treatment modalities includes better stratification of patients into populations of likely responders to a proposed therapy using small molecules capable of inhibiting hyperactive pathways without adverse effects. In addition, supplementing conventional diagnostics with molecular information should help to identify patients with pre-malignant lesions, patients at risk of developing drug resistance, patients with aggressive tumors for whom maximal therapy is appropriate and others who might survive with less toxic adjuvant therapy of reduced intensity (and thus suffer from fewer, less severe side-effects). Therefore, the development of robust and sensitive assays based on biomarkers linked to appropriate chemotherapeutic agents is certainly a need in cancer.

More specifically, there is a need for alternative targeted anti-neoplastic or immune diseases preventions and/or treatments adapted to specific tumor characteristics.

There is also a need for identifying UNG inhibitors to treat AID-associated diseases.

### SUMMARY OF THE INVENTION

The present invention is based in part on the inventors' discovery that failure to faithfully repair off-target DNA deaminations made by the antibody gene diversification enzyme activation-induced deaminase (AID) during an immune response can lead to B cell lymphoma. The inventors have identified a mechanism whereby AID-induced DNA damage and repair at the telomeres can act as a sensor to eliminate B cells at risk of genomic instability. The inventors show that telomeres are off-target substrates of AID and that B cell proliferation depends on protective repair by UNG. In contrast, in the absence of UNG activity, deleterious processing by MMR leads to telomere loss and defective cell proliferation. Indeed we show that UNG-deficiency reduces B cell clonal expansion in the germinal center in mice and blocks the proliferation of tumor B cells expressing AID.

The inventors also show that AID targets and damages telomeric C-rich strand of B-cells and that UNG protects telomeres against this activity in AID-expressing cells, including neoplastic cells. Results disclosed herein indeed show that inhibition of UNG (the glycosylase initiating the most prominent uracil-excision repair pathway) results in the recruitment of MMR proteins to the telomeres, which leads to irreversible acute telomere loss and, in turn, to a cell proliferation defect in normal cells expressing AID and malignant B-cells expressing AID. This reveals that B-cells need UNG to tolerate AID expression. This cellular proliferation defect was also observed in human diffuse large B-cell lymphoma (DLBCL) cells expressing AID. This genetic ablation of UNG increases the incidence of B-cell lymphomas in mice in an AID-independent manner. The present invention takes advantage of a small peptidic UNG inhibitor, namely Ugi, which inhibits UNG by direct binding with high affinity with its active site. The present invention also shows that UNG KO mice and progenies do not display any specific adverse phenotype in normal conditions, save for the small increase in the incidence of lymphoma, which has only a minor effect on the longevity of UNG KO mice compared to WT mice, as shown here. Targeting UNG thus appears to have adverse consequences specifically for AID positive cells (e.g., B-cell lymphomas) and to display low toxicity for AID negative cells (i.e. vast majority of normal body cells except antigen-stimulated B-cells that only transiently express AID during the germinal center reaction). The present invention encompasses the use of UNG inhibitors to promote these AID and/or MMR-related telomere damages in AID expressing pathogenic cells such as B cell lymphoma cells (e.g., DLBCL), leukemia and other AID expressing pathogenic cells.

Without being limited by this hypothesis, the invention submits that while UNG protect cells against mutations by AID, AID's cytotoxic effect on telomeres, and its consequent contribution to AID-associated neoplastic diseases, prevails in UNG's absence in view of data presented herein.

More specifically, in accordance with an aspect of the present invention, there is provided a method for the prevention and/or treatment of an AID-associated disease in a subject in need thereof, said method comprising administering an effective amount of an uracil-DNA glycosylase (UNG) inhibitor to a subject having AID expressing neoplastic cells.

In a specific embodiment, the AID-associated disease is an AID-associated neoplastic disease. In another specific embodiment, the AID-associated neoplastic disease is a B-cell lymphoma or leukemia. In another specific embodiment, the method further comprises detecting the level and/or activity of the AID expressing neoplastic cells in the subject. In another specific embodiment, the UNG inhibitor is an Ugi peptide. In another specific embodiment, the method further comprises administering at least one further therapeutic agent to the subject. In another specific embodiment, the at least one further therapeutic agent comprises at least one compound that favors AID nuclear localization. In another specific embodiment, the compound that favors AID nuclear localization is an eukaryotic elongation factor 1 α (eEF1A) inhibitor. In another specific embodiment, the eEF1A inhibitor is didemnin B (DidB) or cytotrienin A (CytA)). In another specific embodiment, the at least one further therapeutic agent comprises at least one mismatch repair (MMR) pathway stimulator.

In accordance with another aspect of the present invention, there is provided a kit for preventing and/or treating an AID-associated disease in a subject comprising an uracil-DNA glycosylase (UNG) inhibitor and at least one further therapeutic agent.

In a specific embodiment of the method and/or kit, the AID-associated disease is an AID-associated neoplastic disease. In another specific embodiment, the AID-associated neoplastic disease is a B-cell lymphoma or leukemia. In another specific embodiment, the method further comprises detecting the presence, level and/or activity of the AID expressing neoplastic cells in the subject or the kit comprises ligands for detecting the presence, level and/or activity of the AID expressing neoplastic cells. In another specific embodiment, the method further comprises detecting the presence, level and/or activity of UNG expressing neoplastic cells in the subject and the kit comprises ligands for detecting the level and/or activity of UNG expressing neoplastic cells. In another specific embodiment, the method further comprises detecting the presence, level and/or activity of MMR (e.g., MSH2, MSH3, MSH6, MLH1, PMS2, exonuclease 1 (EXO1) or a combination of at least two, at least three, at least four, at least 5, at least 6 thereof) expressing neoplastic cells in the subject and the kit comprises ligands for detecting the presence, level and/or activity of the MMR expressing neoplastic cells.

In another specific embodiment, the method further comprises detecting the presence, level and/or activity of AID in neoplastic cells in the subject and the kit comprises ligands for detecting the presence, level and/or activity of AID in neoplastic cells. In another specific embodiment, the method further comprises detecting the presence, level and/or activity of UNG in neoplastic cells in the subject and the kit comprises ligands for detecting the presence, level and/or activity of UNG in neoplastic cells. In another specific embodiment, the method further comprises detecting the presence, level and/or activity of a functional MMR pathway in neoplastic cells in the subject and the kit comprises ligands for detecting the presence, level and/or activity of a functional MMR pathway in neoplastic cells. In another specific embodiment of the methods and kits, the subject expresses AID in neoplastic cells. In another specific embodiment of the methods and kits, the subject expresses UNG in neoplastic cells. In another specific embodiment of the methods and kits, the subject expresses a functional MMR pathway in neoplastic cells.

In another specific embodiment, the UNG inhibitor is an Ugi peptide. In another specific embodiment, the method further comprises administering at least one further therapeutic agent to the subject and/or the kit further comprises at least one further therapeutic agent. In another specific embodiment, the at least one further therapeutic agent comprises at least one compound that favors AID nuclear localization. In another specific embodiment, the compound that favors AID nuclear localization is an eukaryotic elongation factor 1 α (eEF1A) inhibitor. In another specific embodiment, the eEF1A inhibitor is didemnin B (DidB) or cytotrienin A (CytA)). In another specific embodiment, the at least one further therapeutic agent comprises at least one mismatch repair pathway stimulator.

There is also provided a method for stratifying a subject, said method comprising: measuring the UNG expression and/or activity in a first sample of the subject, and comparing said expression and/or activity to a reference UNG expression and/or activity, wherein an UNG expression and/or activity in the first sample of the subject that is higher than the reference UNG expression and/or activity is indicative that the subject would benefit from a treatment with at least one UNG inhibitor.

There is also provided a method for stratifying a subject, said method comprising: measuring the UNG expression and/or activity in a sample of the subject, and the presence of an UNG expression and/or activity in the sample is indicative that the subject would benefit from a treatment with at least one UNG inhibitor. In a more specific embodiment, the UNG expression and/or activity corresponds to about 10% of a reference UNG expression and/or activity. In a more specific embodiment, it corresponds to about 20%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 160%, about 170%, about 180%, about 190%, about 200%, or more of the a reference UNG expression and/or activity.

In a specific embodiment of the stratification methods, the methods further comprise measuring AID expression and/or activity in a sample of the subject, wherein the presence of an AID expression and/or activity in the sample is indicative that the subject would further benefit from a treatment with at least one UNG inhibitor. In a specific embodiment, the AID expression and/or activity corresponds to about 10% of a reference AID expression and/or activity. In a more specific embodiment, it corresponds to about 20%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 160%, about 170%, about 180%, about 190%, about 200%, or more of the a reference AID expression and/or activity.

In a more specific embodiment of the stratification methods, the methods further comprise measuring AID expression and/or activity in a first sample of the subject, and comparing said expression and/or activity to a reference AID expression and/or activity, wherein an AID expression and/or activity in the first sample of the subject that is substantially similar to or higher than the reference AID expression and/or activity is indicative that the subject would further benefit from a treatment with at least one UNG inhibitor. In a more specific embodiment, substantially similar or higher AID expression and/or activity corresponds to about 90%, about 95%, about 100%, about 105%, about 110%, about 115%, about 120%, about 125%, about 130%, about 135%, about 140%, about 145%, about 150%, about 160%, about 170%, about 180%, about 190%, about 200%, or more of the reference AID expression and/or activity. In a specific embodiment of the stratification methods, when the AID expression in the first sample of the subject is substantially similar to the reference AID expression, the method further comprises the step of: detecting in the first or a second sample of the subject the presence of a loss-of-function mutation, or a deficient expression and/or activity, in at least one gene known to regulate AID mutator activity by controlling or repairing DNA damage, wherein the presence of a mutation in the at least one gene in the first or second sample of the subject is indicative that the subject would further benefit from a treatment with at least one UNG inhibitor.

In another specific embodiment of the stratification methods, the methods further comprise measuring MMR expression and/or activity in a first sample of the subject, and the presence of an MMR expression and/or activity is indicative that the subject would benefit from a treatment with at least one UNG inhibitor. In a more specific embodiment, the MMR expression and/or activity corresponds to about with at least one UNG inhibitor. In a specific embodiment, the MMR expression and/or activity corresponds to about 10% of a reference MMR expression and/or activity. In a more specific embodiment, it corresponds to about 20%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 160%, about 170%, about 180%, about 190%, about 200%, or more of the reference MMR expression and/or activity.

In accordance with another aspect of the present invention, there is provided a method for the prevention and/or treatment of an AID-associated neoplastic disease in a subject in need thereof, said method comprising: measuring the level of AID expression and/or activity and/or UNG expression and/or activity in a first sample from the subject, comparing said expression and/or activity to a reference AID expression and/or activity and/or UNG, wherein, if the AID expression and/or activity and/or UNG is higher in the first sample from the subject than the reference AID expression and/or activity and/or UNG, an effective amount of an UNG inhibitor is administered to the patient.

In accordance with another aspect of the present invention, there is provided a method for the prevention and/or treatment of an AID-associated neoplastic disease in a subject in need thereof, said method comprising: detecting the presence of (i) AID expression and/or activity; and/or (ii) UNG expression and/or activity; and/or (iii) MMR expression and/or activity in a sample from the subject, wherein, when the presence of (i) the AID expression and/or activity; and/or of (ii) the UNG expression and/or activity; and/or of (iii) the MMR expression and/or activity is detected in the sample from the subject, an effective amount of an UNG inhibitor is administered to the patient. In a more specific embodiment, the method comprises detecting the presence of (i) AID expression and/or activity; and (ii) UNG expression and/or activity in the sample from the subject, wherein, when the presence of (i) the AID expression and/or activity; and of (ii) the UNG expression and/or activity is detected, an effective amount of an UNG inhibitor is administered to the patient. In a more specific embodiment, the method comprises detecting the presence of (i) AID expression and/or activity; and (iii) MMR expression and/or activity in the sample from the subject, wherein, when the presence of (i) the AID expression and/or activity; and of (iii) the MMR expression and/or activity is detected, an effective amount of an UNG inhibitor is administered to the patient. In a more specific embodiment, the method comprises detecting the presence of (i) AID expression and/or activity; (ii) UNG expression and/or activity; and (iii) MMR expression and/or activity in the sample from the subject, wherein, when the presence of (i) the AID expression and/or activity; (ii) the UNG expression and/or activity; and of (iii) the MMR expression and/or activity is detected, an effective amount of an UNG inhibitor is administered to the patient.

In a another embodiment of the method, when the AID expression and/or activity; and/or UNG in the first sample of the subject is substantially similar to the reference AID expression and/or activity and/or UNG, the method further comprises the step of: detecting in the first or a second sample of the subject the presence of a loss-of-function mutation, or a deficient expression and/or activity, in at least one gene known to regulate AID mutator activity by controlling or repairing DNA damage, wherein the presence of a mutation in the at least one gene in the first or second sample of the subject is indicative that the subject would benefit from a treatment with at least one UNG inhibitor.

In a another embodiment of the methods, the method further comprises the step of: detecting in the first or a second sample of the subject the presence of a loss-of-function mutation, or a deficient expression and/or activity, in at least one gene known to regulate AID mutator activity by controlling or repairing DNA damage, wherein the presence of a mutation in the at least one gene in the first or second sample of the subject is further ndicative that the subject would benefit from a treatment with at least one UNG inhibitor.

In another specific embodiment, the AID-associated disease is a B-cell lymphoma (e.g., human diffuse large B-cell lymphoma (DLBCL)) or a leukemia (e.g., chronic lymphocytic leukemia (CLL)).

In another specific embodiment, the administration is a monotherapy. In another specific embodiment, the method further comprises administration of at least one other therapy to the subject. In another specific embodiment, the at least one other therapy comprises at least one further antineoplastic therapy.

In another aspect, the present invention provides a method of identifying an agent that may be useful for inhibiting UNG or preventing or treating an AID associated disease, said method comprising contacting a candidate agent with a cell expressing UNG, and determining the UNG expression and/or activity in the presence and in the absence of the agent, wherein a lower or decreased UNG expression and/or activity in the presence of said agent is indicative that said agent may be useful for inhibiting UNG or preventing or treating an AID associated disease. In a specific embodiment, the cell further expresses AID. In another specific embodiment, the cell further expresses mismatch repair pathway enzymes.

The present invention also provides the following:

Item 1. A method for the prevention and/or treatment of an activation-induced deaminase (AID)-associated disease in a subject in need thereof, said method comprising administering an effective amount of an uracil-DNA glycosylase (UNG) inhibitor, or a composition comprising the inhibitor, and a pharmaceutically acceptable carrier, to a subject having pathogenic cells expressing AID, uracil-DNA glycosylase (UNG) and mismatch repair pathway (MMR).
Item 2. The method of item 1, wherein the AID-associated disease is an AID-associated neoplastic disease and the pathogenic cells are neoplastic cells.
Item 3. The method of item 2, wherein the AID-associated neoplastic disease is a B-cell lymphoma or leukemia.
Item 4. The method of any one of items 1 to 3, further comprising detecting (i) AID expression and/or activity; (ii) UNG expression and/or activity; (iii) MMR expression and/or activity; or (iv) a combination of at least two of (i) to (iii) in the pathogenic cells.
Item 5. The method of any one of items 1 to 3, further comprising detecting (i) AID expression and/or activity; (ii) UNG expression and/or activity; and (iii) MMR expression and/or activity in the pathogenic cells.
Item 6. The method of any one of items 1 to 5, wherein the UNG inhibitor is an Ugi peptide.
Item 7. The method of any one of items 1 to 6, further comprising administering at least one further therapeutic agent to the subject.
Item 8. The method of item 7, wherein the at least one further therapeutic agent comprises at least one compound that favors AID nuclear localization.
Item 9. The method of item 8, wherein the compound that favors AID nuclear localization is an eukaryotic elongation factor 1 α (eEF1A) inhibitor.
Item 10. The method of item 9, wherein the eEF1A inhibitor is didemnin B (DidB) or cytotrienin A (CytA)).
Item 11. A method for stratifying a subject having an activation-induced deaminase (AID)-associated disease comprising:
Item 12. detecting AID expression and/or activity;
Item 13. detecting uracil-DNA glycosylase (UNG) expression and/or activity;
Item 14. detecting mismatch repair pathway (MMR) expression and/or activity; or
Item 15. detecting a combination of at least two of (i) to (iii),
Item 16. in the pathogenic cells, wherein said detecting enables the stratification of the subject.
Item 17. The method of item 11, further comprising administering an effective amount of an uracil-DNA glycosylase (UNG) inhibitor to the subject.
Item 18. The method of item 11 or 12, further comprising administering at least one further therapeutic agent to the subject.
Item 19. The method of item 13, wherein the at least one further therapeutic agent comprises at least one compound that favors AID nuclear localization.
Item 20. The method of item 14, wherein the compound that favors AID nuclear localization is an eukaryotic elongation factor 1 α (eEF1A) inhibitor.
Item 21. The method of item 15, wherein the eEF1A inhibitor is didemnin B (DidB) or cytotrienin A (CytA)).
Item 22. A use of an uracil-DNA glycosylase (UNG) inhibitor or of a composition comprising the inhibitor, and a pharmaceutically acceptable carrier, for the prevention and/or treatment of an activation-induced deaminase (AID)-associated disease in a subject in need thereof, wherein the subject has pathogenic cells expressing AID, uracil-DNA glycosylase (UNG) and mismatch repair pathway (MMR).
Item 23. A use of an uracil-DNA glycosylase (UNG) inhibitor or a composition comprising the inhibitor, and a pharmaceutically acceptable carrier, in the manufacture of a medicament for the prevention and/or treatment of an activation-induced deaminase (AID)-associated disease in a subject in need thereof, wherein the subject has pathogenic cells expressing AID, uracil-DNA glycosylase (UNG) and mismatch repair pathway (MMR).
Item 24. The use of item 17 or 18, wherein the AID-associated disease is an AID-associated neoplastic disease and the pathogenic cells are neoplastic cells.
Item 25. The use of item 19, wherein the AID-associated neoplastic disease is a B-cell lymphoma or leukemia.
Item 26. The use of any one of items 17 to 20, wherein (i) AID expression and/or activity; (ii) UNG expression and/or activity; (iii) MMR expression and/or activity; or (iv) a combination of at least two of (i) to (iii) was detected in the pathogenic cells of the subject.
Item 27. The use of any one of items 17 to 20, wherein (i) AID expression and/or activity; (ii) UNG expression and/or activity; and (iii) MMR expression and/or activity were detected in the pathogenic cells of the subjec.
Item 28. The use of any one of items 17 to 22, wherein the UNG inhibitor is an Ugi peptide.
Item 29. The use of any one of items 17 to 23, further comprising the use of at least one further therapeutic agent to the subject.
Item 30. The use of item 24, wherein the at least one further therapeutic agent comprises at least one compound that favors AID nuclear localization.
Item 31. The use of item 25, wherein the compound that favors AID nuclear localization is an eukaryotic elongation factor 1 α (eEF1A) inhibitor.
Item 32. The use of item 28, wherein the eEF1A inhibitor is didemnin B (DidB) or cytotrienin A (CytA)).
Item 33. A composition comprising an uracil-DNA glycosylase (UNG) inhibitor for use in the prevention and/or treatment of an activation-induced deaminase (AID)-associated disease in a subject in need thereof, wherein the subject has pathogenic cells expressing AID, uracil-DNA glycosylase (UNG) and mismatch repair pathway (MMR).
Item 34. The composition for use of item 28, wherein the AID-associated disease is an AID-associated neoplastic disease and the pathogenic cells are neoplastic cells.
Item 35. The composition for use of item 29, wherein the AID-associated neoplastic disease is a B-cell lymphoma or leukemia.
Item 36. The composition for use of any one of items 28 to 30, wherein (i) AID expression and/or activity; (ii) UNG expression and/or activity; (iii) MMR expression and/or activity; or (iv) a combination of at least two of (i) to (iii) was detected in the pathogenic cells of the subject.
Item 37. The composition for use of any one of items 28 to 30, wherein (i) AID expression and/or activity; (ii) UNG expression and/or activity; and (iii) MMR expression and/or activity were detected in the pathogenic cells of the subject.
Item 38. The composition for use of any one of items 28 to 32, wherein the UNG inhibitor is an Ugi peptide.
Item 39. The composition for use of any one of items 28 to 33, wherein the composition further comprises at least one further therapeutic agent.
Item 40. The composition for use of item 34, wherein the at least one further therapeutic agent comprises at least one compound that favors AID nuclear localization.
Item 41. The composition for use of item 35, wherein the compound that favors AID nuclear localization is an eukaryotic elongation factor 1 α (eEF1A) inhibitor.
Item 42. The composition for use of item 36, wherein the eEF1A inhibitor is didemnin B (DidB) or cytotrienin A (CPA)).
Item 43. The composition for use of any one of items 28 to 37, further comprising a pharmaceutically acceptable carrier.
Item 44. A kit for preventing and/or treating an activation-induced deaminase (AID)-associated disease in a subject, comprising an uracil-DNA glycosylase (UNG) inhibitor or a composition comprising the inhibitor, and a pharmaceutically acceptable carrier, and at least one further therapeutic agent.
Item 45. The kit of item 39, wherein the AID-associated disease is an AID-associated neoplastic disease.
Item 46. The kit of item 39 or 40, wherein the UNG inhibitor is an Ugi peptide.
Item 47. The kit of any one of items 39 to 41, wherein the at least one further therapeutic agent comprises at least one compound that favors AID nuclear localization.
Item 48. The kit of item 42, wherein the compound that favors AID nuclear localization is an eukaryotic elongation factor 1 α (eEF1A) inhibitor.
Item 49. The kit of item 43, wherein the eEF1A inhibitor is didemnin B (DidB) or cytotrienin A (CytA)).

### BRIEF DESCRIPTION OF THE DRAWINGS

In the appended drawings:

FIGS. 1A-E. Telomeric association of AID in primary mouse B-cells during CSR. FIG. 1A. Schematic depiction of similarities between telomeres and Ig S-regions and location of AID's preferred sequences (WRCY). Note that WRCY motifs are present in both S-region strands but exclusively in the C-rich strand in telomeres. (SEQ ID NOs: 1, 44 (Sμ repeat) and 2-3 (telomeric repeat). FIG. 1B Bottom, representative dot-blot analysis of ChIP assays using anti-AID and IgG control in stimulated splenic B-cells purified from Aicda+/+ (WT) or Aicda−/− mice and stimulated with LPS and IL-4 for 24 h. ChIP for the telomeric protein TRF1 was included as positive control. Dot blots with the immunoprecipitates were analyzed via Southern blot with telomeric (Tel) or ALU (Alu) repeat probes. Right, Quantification of the ChIPs with antibodies against AID. FIG. 1C Quantification of AID accumulation by ChIP at telomeres or Alu repeats (by dot-blot, panel C) as well as Sμ and Cμ regions of the Igh locus (by Q-PCR) in CH12F3 cells stimulated for CSR. Average+SD values obtained at each time point from three independent experiments are represented. FIG. 1D ChIPs in WT splenic B-cells with the indicated antibodies. Quantification of the Dot-blot signals after Southern blot with a telomeric probe in shown at the right. Fig. 1E Northern blot with a telomeric probe showing the concentration of TERRAs in WT splenic B-cells before and after stimulation of CSR. Right, Quantification of Northern signals. Error bars represent standard error. Average+SD values obtained at each time point from three independent experiments are represented in the graph. ChIPs in CH12F3 B cells with the indicated antibodies. Quantification of the Dot-blot signals after Southern blot with a telomeric probe in shown at the right.

FIGS. 2A-F. Association of AID with the telomer of CH12F3 B-cells stimulated for CSR to IgA. FIG. 2A Western blot analysis of AID expression levels in CH12F3 cells after CSR stimulation. FIG. 2B AID recruitment to the Ig locus during CSR analyzed by ChIPs in CH12F3 B-cells. The position of the different amplicons used for the ChIP assays is indicated in the graph. FIG. 2C Representative dot-blot analysis of ChIP assays using anti-AID and IgG control in stimulated CH12F3B cells. Dot blots with 5% of the input or the immunoprecipitates were analyzed via Southern blot with telomeric or Alu repeat probes. Right, Quantification of AID signals in the dot blots after Southern analysis. FIG. 2D Left, Western blot analysis of AID expression levels in CH12F3 cells expressing the indicated shRNAs. Right, As in FIG. 2C but in CH12F3 cells with the indicated antibodies. Error bars represent standard error. Co-immunoprecipitated telomeric DNA was detected via Southern blot with a telomeric probe in Dot-blots Average+SD values obtained at each time point from three independent experiments are represented in the graph. FIG. 2E ChIPs in CH12F3 B cells with the indicated antibodies. Quantification of the Dot-blot signals after Southern blot with a telomeric probe in shown at the right. FIG. 2F Northern blot with a telomeric probe showing the concentration of TERRAs in wt splenic B cells before and after stimulation of CSR. Right, Quantification of Northern signals. Average+SD values obtained at each time point from three independent experiments are represented in the graph.

FIGS. 3A-F. AID-dependent telomeres loss in UNG-deficient B-cells. FIG. 3A Possible outcomes after processing of AID-dependent DNA deaminations by UNG in B cells. FIG. 3B Left, illustration of typical FISH staining with a telomere-specific probe in metaphase chromosomes from normal cells and cells with sister telomere loss (STL). Right, effect of UNG inhibition on the number of metaphases with STL in different CH12F3 lines expressing scrambled (scr) control or two different shRNAs that deplete AID, before and after stimulation of CSR to IgA. FIG. 3C Top, representative pictures of metaphase chromosomes FISH in wt, Ung−/−, and Ung−/− Aicda−/− mouse splenic B cells stimulated for CSR to IgG1. Telomeres were hybridized with a Alexa488-[TTAGGG]₄ probe and are visualized as bright dots at the end of the chromosome arms; total DNA was stained with 4,6-diamidino-2-phenylindole (DAPI). Arrows indicate missing telomere staining from single sister chromatids. Bottom, Quantification of STL per metaphase and percentage of metaphases with STL. FIG. 3D Schematic of CO-FISH. Leading strand (C-rich) telomeres are shown in darker grey and lagging strand (G-rich) telomeres in paler grey at the end of the chromosome arms. FIG. 3E Left, FISH analysis of metaphase chromosomes of CH12F3 cells expressing Ugi and full-length AID or deaminase-dead AID mutant (AIDE58A) as indicated. Right, quantification of STL in the different CH12F3 cell lines without stimulation of class switching. FIG. 3F Left, illustration of COFISH staining. Leading-strand telomeres are darker and lagging-strand telomeres paler. Center. representative pictures of COFISH in wt, Ung−/−, and Ung−/− Aicda−/− splenic B-cells at 4 d post-stimulation with LPS and IL-4. Right, quantification of STL per metaphases after CO-FISH analysis in different cell lines. In (B), (D) and (E) bars indicate average+SD from three independent experiments.

FIGS. 4A-C. 4A Representative FISH analysis of metaphase chromosomes of wt, Ung−/−, and Ung−/− Aicda−/− splenic B-cells stimulated for CSR to IgG1. Telomeres were hybridized with a Alexa488-[TTAGGG]₄ probe and are shown as bright dots at the end of the chromosome arms; total DNA was stained with 4,6-diamidino-2-phenylindole (DAPI). 4B Examples of CO-FISH analysis of metaphase chromosomes of wt, Ung−/−, and Ung−/− Aicda−/− splenic B cells stimulated during days for CSR to IgG1. In FIGS. 5A-B, arrows indicate STL. FIG. 4C Left, Western analysis of wt AID or a deaminase-dead mutant version of AID (AIDE58A) levels in CH12F3 Ugi cells. Right, Quantification of metaphases with STL from CH12F3 Ugi cells expressing GFP, AID or AIDE58.

FIGS. 5A-B. UNG-deficiency does not affect the average telomere length. FIG. 5A Diagram showing the terminal restriction fragment (TRF) analysis of TTAGGG repeats via Southern blot in native or denatured condition TRF analysis of genomic DNA from wt or Ung^{-/-} splenic B cells before and after stimulation of CSR to IgG1. FIG. 5B Ratio between native and denatured signals from telomeric Southern analysis. The mean and standard deviation of two experiments is shown.

FIGS. 6A-B. FIG. 6A Quantification of STL levels in CH12F3 and CH12F3 Ugi cells expressing the indicated shRNAs before and after stimulation of CSR to IgA. FIG. 6B COFISH in metaphase chromosomes from CH12F3 and CH12F3 Ugi cells expressing the indicated shRNAs. Right, Quantification of STL levels in the indicated cell lines.

FIGS. 7A-H. MMR proteins mediate AID-Induced STL in in UNG-deficient B-cells expressing AID. FIG. 7A Possible outcomes of MSH2/MSH6-initiated repair of AID-induced DNA deaminations in B cells. FIG. 7B Western analysis of MSH2 knockdown in CH12F3 cells expressing the indicated shRNAs. FIG. 7C Quantification of metaphase chromosomes with STL in different CH12F3 cell lines expressing or not Ugi and scrambled (scr) control or or two different shRNAs that deplete MSH2, before and after stimulation of CSR to IgA. STL average+SD values obtained at each time point from three independent experiments are represented in the graph. FIG. 7D A fluorescein-labeled oligonucleotide containing a single dU residue was incubated with cell extracts (10 [symbol] g protein) from CH12F3 lines expressing or not Ugi and scrambled (scr) control or two different shRNAs that deplete MSH2. The reaction products were resolved on 15% TBE-urea polyacrylamide gels and are indicated at the left. Western analysis of γ-Tubulin level was used as loading control. FIG. 7E Left, representative ChIP performed with the indicated antibodies in wt, Ung−/−, and Ung−/− Aicda−/− splenic B-cells stimulated for CSR to IgG1 and analyzed by Southern dot-blot using telomeric or Alu-probes. Right, Plot of the average+SD dot-blot signals for the telomeric probe from three independent experiments. FIG. 7F Terminal restriction fragment (TRF) analysis of TTAGGG repeats in stimulated CH12F3 and CH12F3-Ugi cells expressing the indicated shRNAs via Southern blot in native or denatured conditions. FIG. 7G Diagram showing the expected outcomes after treatment of genomic DNA with Exonuclease I before the TRF analysis of TTAGGG repeats. The 3′-5′ single-strand exonuclease activity of Exol will remove the telomeric 3′ G-rich overhang, therefore the signal for single-stranded TTAGGG repeats will be lost in a TRF analysis in native conditions. However in telomeres with short gaps or nicks in the C-rich strand Exol activity will expose G-rich single-stranded gaps that can be detected in a TRF analysis in native conditions. FIG. 7H Left, representative southern blots of TRF after Exol treatment in native and denatured conditions. Right, quantification of telomeric ssDNA/dsDNA ratio in Exol-treated genomic DNA from 3 experiments.

FIG. 8. P53 deletion increases the number of metaphases with STL in activated UNG-null B-cells. Quantification of STL levels in p53−/−, Ung−/−, or Ung−/− p53−/− after stimulation of class switching to IgG1. Error bars represent standard error from two independent experiments.

FIGS. 9A-C. Compromised Proliferation of UNG-Deficient B Cells Expressing AID. FIG. 9A Left, representative microscopy pictures of anaphases from CH12F3 cells expressing the HPV16 E6 and E7 oncoproteins and GFP control, Ugi, or Ugi shAID. Total DNA was stained with DAPI (shown pale). Right, Quantification of anaphases showing chromosome bridges in CH12F3 GFP and CH12F3 Ugi cells. FIG. 9B Cell cycle profile analysis by bromodeoxyuridine (BrdU) incorporation and propidium iodide (PI) staining in the indicated CH12F3 cells 24 h after stimulating CSR. FIG. 9C Cell number (left) and CFSE staining (right) were used to evaluate cellular proliferation of CH12F3 and CH12F3 Ugi cells expressing the indicated shRNAs after stimulation for CSR. Bars in FIG. 12B and lines in FIG. 12C represent averages+SD from at least three independent experiments.

FIGS. 10A-B. Compromised clonal expansion of Ung deficient GC B-cells expressing AID in vivo and in vitro. FIG. 10A Top, absolute number and, Bottom, proportion of AID-GFP+ cells in the spleen of AID-GFPtg and Ung−/− AID-GFPtg mice 8 days after immunization with 50 ug NP-CGG. Results from 3 independent experiments are compiled. P values from unpaired two-tailed t-test are shown. FIG. 10B Clonal proliferation of CH12F3, a mouse lymphoma B cell line, and CH12F3 Ugi cells, CH12F3 cells expressing Ugi,and expressing the indicated shRNAs after stimulation of class switching to IgA. Error bars represent SD from at least three independent experiments. t test *P=0.001, **P=0.02. FIG. 10C Representative confocal images at 40× magnification of AID-GFP₊ GC B cells in the spleen of AID-GFPtg and Ung^{-/-} AID-GFPtg mice. Bars are 50 [symbol] m. FIG. 10D Left, each symbol represents the median area in square inches (sq in) of all GC observed in splenic sections from 4 wt and 5 Ung^{-/-} AID-GFPtg mice coming from two of the experiments represented in FIG. 10A. P values using unpaired two-tailed t-test. Right, the number of GC per spleen were counted in the same mice as in the left panel. Horizontal bars indicate median values.

FIGS. 11A-E. The Proliferation of Malignant B Cells Expressing AID Depends on UNG. FIG. 11A Kaplan-Meier survival curve of C57BL6/J WT (Ctrl) (n=23), Ung−/−(n=29), Aicda−/−(n=30,) and Ung−/− Aicda−/− (n=39) mice (WT vs Aicda−/−, p=0.0011; WT vs Ung−/− Aicda−/−, p=0.0002 by Log-rank test, α=0.05, all other comparisons are not significant). FIG. 11B Median lifespan of the mice in A. Statistically significant differences by one-way ANOVA with Dunnett's post-test are indicated. FIG. 11C Incidence of spontaneous lymphoma or other cancers in the indicated mouse cohorts. Statistical significant differences in the proportions by Fisher exact test, α=0.05, are indicated. FIG. 11D Lymphoma spectrum and median lifespan of mice diagnosed with lymphomas from the cohorts in B. BCL, B-cell lymphoma; TCL, T-cell lymphoma. FIG. 11E Incidence of spontaneous lymphoma, lymphoid hyperplasia, or any other tumor in wt and Ung^{-/-} mice. The total number of mice analyzed is shown at the center of each pie.

FIGS. 12A-C. Characterization of lymphomas from UNG-deficient mice. FIG. 12A Representative pictures of H&E staining and IHC with anti-B220 and anti-AID of secondary lymphoid organs from Ung−/− mice that were diagnosed with lymphoma. Pictures magnification, 20×. Flow cytometry analyses of the lymphocytes in that organ using the indicated surface markers are shown below each mouse, with proportion of each gate indicated. LN: lymph nodes. FIG. 12B Top, representative IHC pictures of AID staining in B cell lymphomas found in Ung^{-/-} mice. Positive and negative staining controls are shown using spleen from immunized wt and Aicda^{-/-} mice, respectively. Bottom, AID status of the lymphoma cells, as judged from IHC, for each of the mice in FIG.. 1E diagnosed with B cell lymphoma. FIG. 12C Western blot analysis indicating the AID expression levels in activated (Act) and control (CTL) mouse splenic B cell lines and a B cell lymphoma (BCL) from Ung^{-/-} mouse.

FIGS. 13A-D. UNG and MSH2 control the proliferation of human lymphoma cells expressing AID. FIG. 13A Western blot analysis indicating of AID levels in lymphoma human DLBCL cell lines and control fibroblasts (IMR90). FIG. 13B The proliferation of the human DLBCL cell lines from FIG. 12A expressing GFP control, AID or Ugi and the indicated shRNAs was measured by growth curves. FIG. 13C Quantification of metaphases with STL in different DLBCL lines expressing GFP or Ugi, and AID or shAID as indicated. FIG. 13D A fluorescein-labeled oligonucleotide containing a single dU residue was incubated with cell extracts (10 μg protein) from the indicated DLBCL cell lines. The reaction products were resolved on 15% TBE-urea polyacrylamide gels and are indicated at the left. Western analysis of γ-Tubulin was used as loading control.

FIG. 14 AID Expression In Ung^{-/-} Lymphoma. Representative pictures of histological sections of lymphomas found in Ung^{+/+} and Ung^{-/-} mice, stained by IHC with anti-B220 and anti-AID. Splenic GC from an immunized wt mouse that were stained in parallel are shown as comparison.

FIG. 15 Proposed Molecular Mechanism of the Alternative Processing of Telomeric Damage Induced by AID in B Cells. AID is induced in activated B cells and targets the telomeres concomitantly with CSR. UNG initiates error-free BER of the C-rich telomeres deaminated by AID. In UNG-deficient B cells stimulated for CSR the dG:dU mismatches are recognized by mismatch repair (MMR) and processed into a nick or short gap that could stall the leading strand synthesis and produce a very short telomere in one sister chromatid. Therefore the activity of MMR could limit the proliferation of B cells expressing AID provided UNG activity is eliminated.

FIGS. 16A-D shows the effects of combination DidB treatment and UNG-deficiency on AID toxicity in primary B cells. FIG. 16A Primary B cells were harvested from AID UNG double deficient mice and cultured with anti-CD180, IL-4 and LPS. 24 h later cells were infected with either GFP alone, AID-ires-GFP or AIDE58A-ires-GFP (a catalytically inactive AID variant). 24 h post infection, cells were treated with either DMSO vehicle or 1 nM DidB. Toxicity was measured after 72 h as the % difference of GFP+ cells in the DidB-treated culture compared to DMSO control. Data from two mice are averaged, bars are SEM. The experiment shows that UNG-deficient B cells are substantially more sensitive to DidB when AID is expressed. FIGS. 16B-D B cells were purified from wt or Ung^{-/-} mice, and cultured directly (FIG. 16B) with LPS and IL-4 or FIGS. 16C and D after loading with the CFSE dye. 24 h later, cells were treated with either DMSO or DidB. (FIG. 16B) Cells were counted 48 h post-treatment in order to measure B cell expansion. FIGS. 16C-D Cell divisions for treated cells were measured by analyzing the dilution of CFSE dye at 24 h post-treatment. Dilution of CFSE in cells is proportional to proliferation.

FIGS. 17A-L show nucleotide and amino acid sequences: 17A. Homo sapiens UNG, transcript variant 1, mRNA (NM_003362.3) (SEQ ID NO: 4); 17B. Homo sapiens UNG, transcript variant 1, encoded protein (NP_003353.1) (SEQ ID NO : 5); 17C. Homo sapiens UNG, transcript variant 2, mRNA (NM_080911.2)(SEQ ID NO : 6); 17D. Homo sapiens UNG, transcript variant 2, encoded protein (NP_550433.1)(SEQ ID NO : 7); 17E. Alignment of encoded protein of UNG transcript variants 1 and 2 shown in FIGS. 17B and D and resulting consensus (SEQ ID NO: 8); 17F. Homo sapiens AID nucleotide sequence (cDNA) (SEQ ID NO: 9, genebank accession NM_020661); 17G. Homo sapiens AID amino acid sequence (SEQ ID NO: 10, UniProtKB/Swiss-Prot Q9GZX7-1); 17H. Genebank™ information about Homo sapiens AID providing the amino acid sequence (SEQ ID NO: 11) and full gene sequence (SEQ ID NO: 12); 17I. Homo sapiens mutS homolog 2 (MSH2), transcript variant 1, mRNA (NM_000251) (SEQ ID NO: 13); 17J. Homo sapiens mutS homolog 2 (MSH2), transcript variant 1, encoded protein (NP_000242.1) (SEQ ID NO: 14); 17K. Homo sapiens mutS homolog 2 (MSH2), transcript variant 2, mRNA (NM_001258281) (SEQ ID NO: 15); 17L. Homo sapiens mutS homolog 2 (MSH2), transcript variant 2, encoded protein (NP_001245210.1) (SEQ ID NO: 16); 17M Homo sapiens mutS homolog 3 (MSH3), mRNA, (NM_002439.4) (SEQ ID NO: 17); 17N Homo sapiens mutS homolog 3 (MSH3), mRNA, (NP_002430.3) (SEQ ID NO: 18); 170 Homo sapiens mutS homolog 6 (MSH6), transcript variant 1, (NM_000179.2) (SEQ ID NO: 19); 17P Homo sapiens mutS homolog 6 (MSH6), transcript variant 1, encoded protein (NP_000170.1) (SEQ ID NO: 20); 17Q Homo sapiens mutL homolog 1 (MLH1), transcript variant 1, mRNA (NM_000249) (SEQ ID NO: 21); 17R Homo sapiens mutL homolog 1 (MLH1), transcript variant 1, encoded protein (NM_000240.1) (SEQ ID NO: 22); 17S Homo sapiens PMS1 homolog 2, mismatch repair system component PMS2, transcript variant 1, mRNA (NM_000535.6) (SEQ ID NO: 23); 17T Homo sapiens PMS1 homolog 2, mismatch repair system component PMS2, protein (NP_000526.2) (SEQ ID NO: 24); 17U Homo sapiens exonuclease 1 (EXO1), isoform a, mRNA (NM_003686.4) (SEQ ID NO: 25); 17V Homo sapiens exonuclease 1 (EXO1), isoform a, protein (NP_003677.4) (SEQ ID NO: 26); 17W Homo sapiens exonuclease 1 (EXO1), isoform c, mRNA (NM_001319224.1) (SEQ ID NO: 27); 17X Homo sapiens exonuclease 1 (EXO1), isoform c, protein (NP_001306153.1) (SEQ ID NO: 28).

### DESCRIPTION OF ILLUSTRATIVE EMBODIMENTS

UNG activity/expression inhibition which in turn promotes AID associated telomere damages and tumor cell proliferation are described as well as methods for the stratification of subjects and methods for the prevention and treatment of AID-associated neoplastic diseases.

Neoplastic diseases and AID

The terminology “neoplastic disease” or “invasive disease” is meant herein to refer to a disease associated with new growth of any body tissue. A neoplastic tissue according to the invention is derived from a pre neoplastic tissue and may retain some characteristics of the tissue from which it arises but has biochemical characteristics that are distinct from those of the parent tissue. The tissue formed due to neoplastic growth is a mutant version of the original tissue and appears to serve no physiologic function in the same sense as did the original tissue. It may be benign or malignant (e.g., cancer).

Cancer is defined herein as a disease characterized by the presence of cancer cells which possess two heritable properties: they and their progeny are able (1) to reproduce unrestrained in defiance of the normal restraints (i.e., they are neoplastic) and (2) invade and colonize territories normally reserved for other cells (i.e., they are malignant). Invasiveness of cancer cells usually implies an ability to break loose, enter the bloodstream or lymphatic vessels, and form secondary tumors, or metastases at the other distant sites in the body. The term “cancer cells” refers herein to a cluster of cancer or tumor cells showing over proliferation by non-coordination of the growth and proliferation of cells due to the loss of the differentiation ability of cells. The terms “cancer cell” and “tumor cell” are used interchangeably herein.

AID is not systematically expressed in all cancers nor in all tumors or cells of a defined cancer type. For example, AID expression variations were observed amongst B-cell lymphomas such as DLBCL, gastric adenocarcinomas and cholangiocarcinomas. In another example, CML cells in lymphoid blast crisis (fatal within weeks and months) as opposed to chronic phase (indolent chronic phase standing for years), express AID at high levels. Also, only a fraction of B-chronic lymphocytic leukemia (B-CLL) cells express AID, which is associated with poor prognosis.

Furthermore, an aberrant AID expression and/or activity in a human tissue can be indicative that said tissue may become neoplastic and/or progress to a malignant/cancerous state. It may thus be desirable to inhibit aberrant AID expression and/or activity in subjects having or susceptible to develop a neoplastic disease.

Genes regulating AID mutator activity in B cells by controlling or repairing DNA damage

The AID mutator activity is modulated by several genes known to control/prevent/repair AID-mediated mutations and/or AID-mediated antibody diversification. Amongst them, protein 53 (p53), ataxia telangiectasia mutated (ATM), Nijmegen breakage syndrome 1 (Nbs1) and Alternate-reading-frame tumor suppressor (p19(Arf), p53 upregulated modulator of apoptosis (PUMA), bcl-2 interacting mediator of cell death (Bim) and protein kinase C, delta (PKCdelta) are involved in the control of DNA damage, genomic instability checkpoints and induction of apoptosis. Other genes whose deficiency has been shown to have a synergistic effect with the presence of AID on increasing off-target mutations include the DNA repair enzymes that can recognize uracil in DNA. Examples of DNA repair enzymes include uracil DNA-glycosylase (UNG), which starts uracil excision repair; mutS homolog 2 (MSH2) and mutS homolog 6 (MSH6), a mismatch recognition heterodimer that initiates mismatch repair, as well as downstream components of those pathways, such as the DNA polymerase Beta.

Therefore, the deficient expression and/or activity in a B cell population of a gene regulating AID mutator activity by controlling or repairing DNA damage (e.g., a decrease in the p53 DNA damage controlling activity) may be indicative of a predisposition to B cell pathologies due to an increased activity of AID.

B cell leukemias and lymphomas displaying a level of AID expression similar to that observed in normal B cells, combined or not with deficient expression and/or activity of a gene regulating the AID mutator activity by controlling or repairing DNA damage (e.g., a decrease in p53 DNA damage controlling activity) is also indicative that said cancer may progress to a more malignant state or is susceptible to develop resistance to drug treatment due to an increase activity of AID. It may thus be desirable to inhibit AID in those subjects having B cells in which expression and/or activity of genes regulating AID mutator activity is decreased.

The level of expression of genes (RNA and/or protein) regulating the AID mutator activity can be measured using a variety of assays such as those described below for AID.

Alternatively, the detection of a genomic loss-of-function mutation could be used to measure a decrease in the expression and/or activity of the genes regulating the AID mutator activity by controlling or repairing DNA damage (e.g., loss-of-function mutation at the p53 locus). Genetic loss-of-function mutations are DNA modifications (e.g., deletions, missense substitutions) leading to a decrease in expression and/or activity of a specific gene. For instance, the TP53 (tumor protein 53) gene is the most frequently mutated gene in sporadic cancers. Germline mutations have also been reported in over 500 cancer-prone families. Both somatic and germline mutations are compiled in a worldwide database at the International Agency for Research on Cancer. Most p53 loss-of-function mutations result in missense substitutions that are scattered throughout the gene but are particularly dense in exons 5-8, encoding the DNA binding domain.

Several well-known examples of loss-of-function mutations in genes regulating the AID mutator activity by controlling or repairing DNA damage were reported. Chronic lymphocytic leukaemia (CLL) is a genetically heterogeneous disease. As detected by the interphase cytogenetic fluorescence in situ hybridization (FISH) approach, the most frequent genetic alterations in the prognosis of B-cell chronic lymphocytic leukemia (B-CLL) patients involve deletions in 17p13 (TP53) and 11q22-q23 (ATM). The importance of studying p53 pathway defects in chronic lymphocytic leukemia (CLL) has been promoted by the demonstration of the fundamentally different clinical course of patients with 17p deletion. The observation of resistance to chemotherapy and mutation of the remaining TP53 allele explains the clinical presentation of CLL with 17p deletion.

The most relevant techniques used for detection of genetic alterations in B cells include, amongst others, comparative genomic hybridization (CGH) and FISH, as well as PCR-based techniques coupled with DNA sequencing or multiplex ligation-dependent probe amplification (MLPA) analyses.

AID and cancer progression

Several papers show that in several cancer types (e.g., CML, ALL and B-CLL), AID expression and poor prognosis correlate. One paper also showed AID expression during progression in follicular lymphoma FL suggesting that AID+clones may outgrow the population and that those cases have more advanced states of the disease.

### Autoimmunity and AID

Autoimmunity encompasses a broadly defined area of clinical pathologies that stem from abnormalities in numerous systemic, cellular, and molecular mechanisms, a subset of which are B cell-related autoimmune. In systemic lupus erythematosus, abnormalities in B cell development and the production of autoreactive antibodies play an important pathological role. Overexpression of AID in autoimmune-prone mice induced a more severe systemic lupus erythematosus-like phenotype, whereas breeding AID-deficient mice with autoimmune-prone MRL/Ipr mice significantly reduced the onset and extent of disease, indicating that alterations in AID can change the severity of B cell autoimmunity. Without being so limited, there are several hypotheses on how unregulated AID can affect autoimmunity in addition to overstimulation of SHM and CSR, e.g., debilitating mutations in the signaling pathways, inactivation of tumor suppressors or proapoptotic genes, or alterations that activate oncogenes or antiapoptotic genes.

As used herein, “Autoimmune disease” refers to illnesses that occur when the body tissues are attacked by its own immune system. The immune system is a complex organization within the body that is designed normally to “seek and destroy” invaders of the body, including cancer cells. Patients with autoimmune diseases frequently have unusual antibodies circulating in their blood that target their own body tissues. Examples of autoimmune diseases include Systemic Lupus Erythematosus (SLE), Sjogren syndrome, Hashimoto thyroiditis, Rheumatoid Arthritis (RA), juvenile (type 1) diabetes, polymyositis, scleroderma, Addison disease, vitiligo, pernicious anemia, glomerulonephritis, Multiple Sclerosis (MS), Crohn's disease and pulmonary fibrosis. Autoimmune diseases are more frequent in women than in men. It is believed that the estrogen of females may influence the immune system to predispose some women to autoimmune diseases. Autoimmune diseases that occur more frequently in women than men include RA and SLE. The Relapsing-Remitting and Secondary Progressive forms of MS are nearly twice as common in women as in men although the Primary Progressive form is equally common in men as women.

An AID expression and/or activity in human B cells may be indicative of a predisposition to develop an autoimmune disease.

AID is necessary for CSR to IgE, the immunoglobulin that mediates allergy. It would therefore be useful to administer UNG inhibitors to inhibit AID and in turn reduce production of IgE, thus reducing the severity of atopic allergic reactions.

Normal AID expression in B cells combined or not to a deficient expression and/or activity of genes regulating AID mutator activity by controlling or repairing DNA damage may also be indicative of predisposition to autoimmune disease.

UNG gene and UNG protein

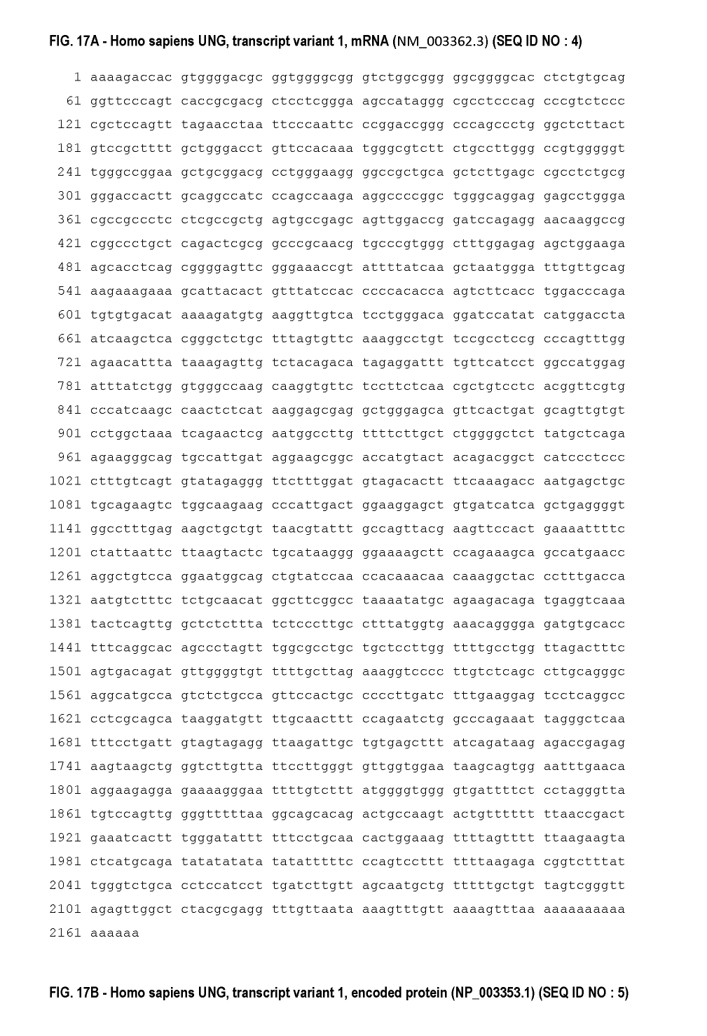

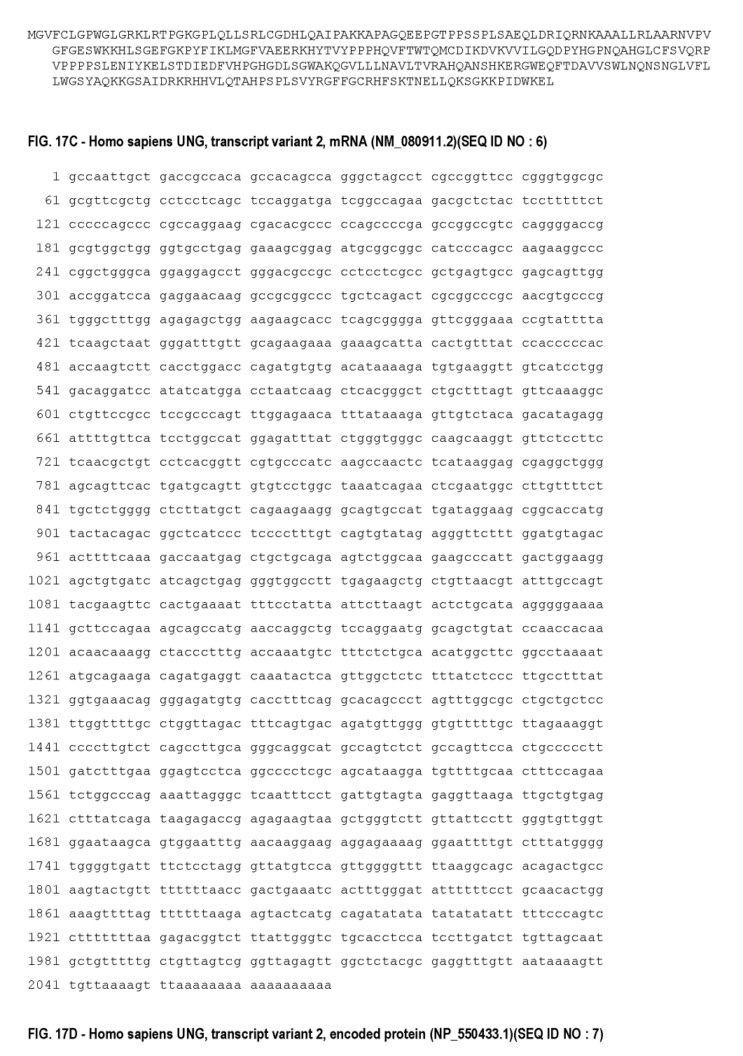

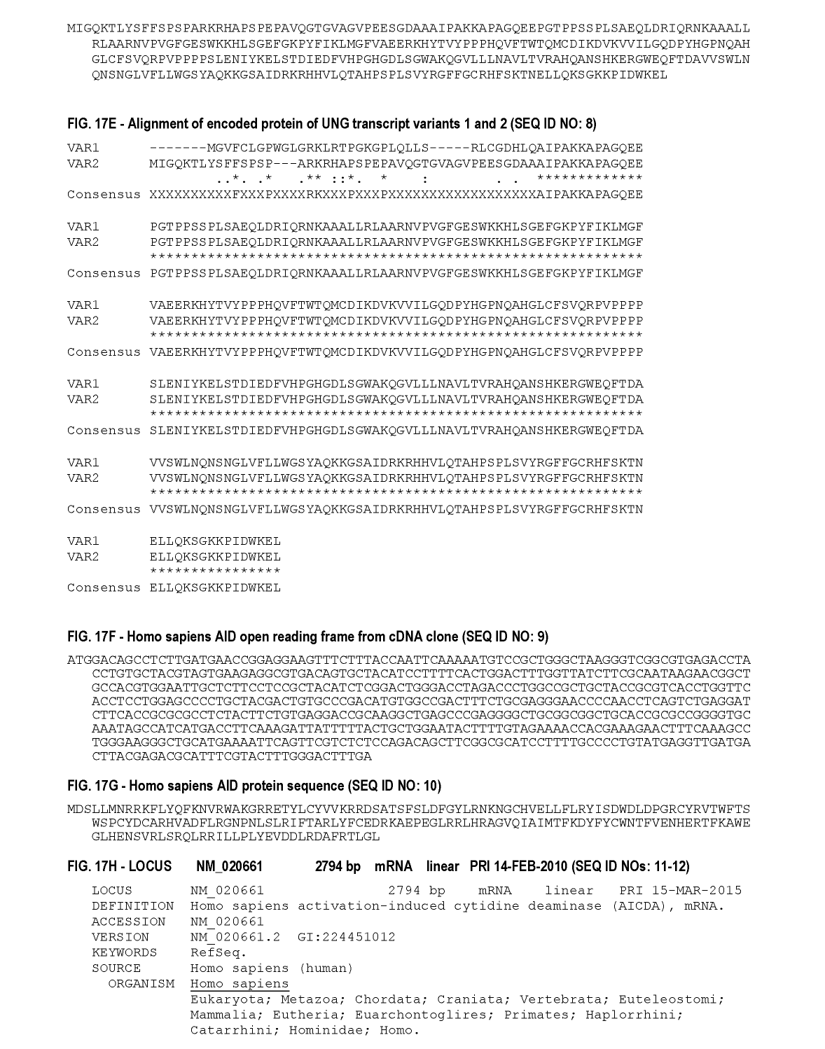

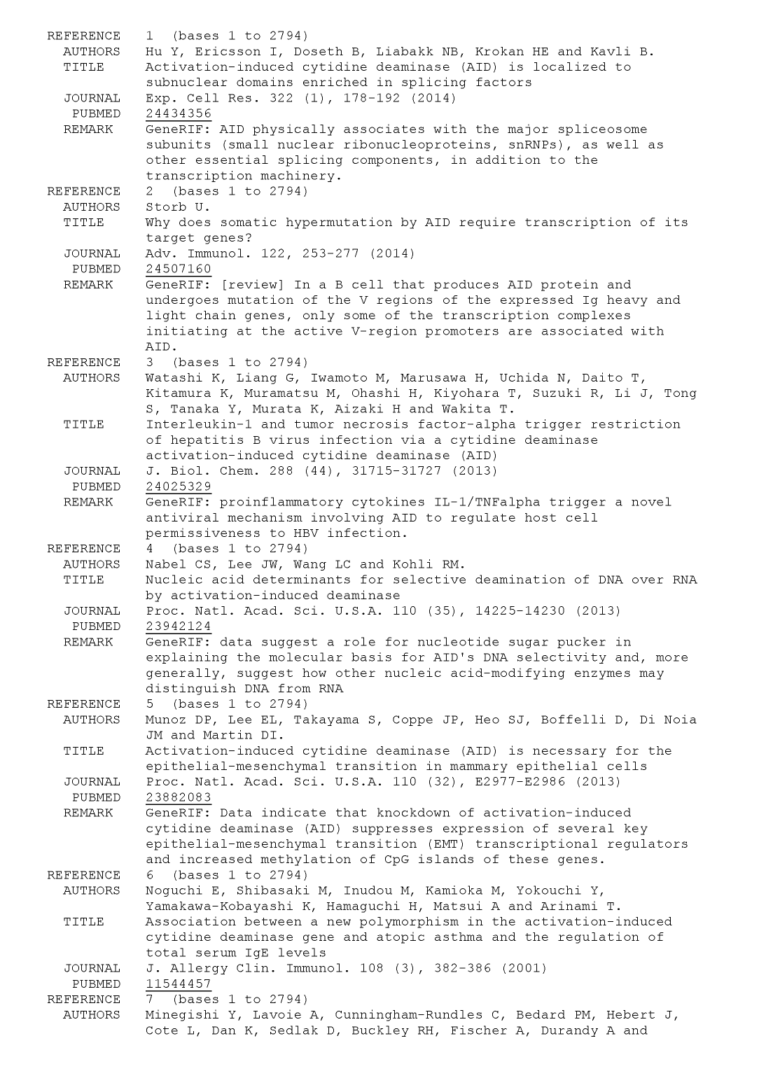

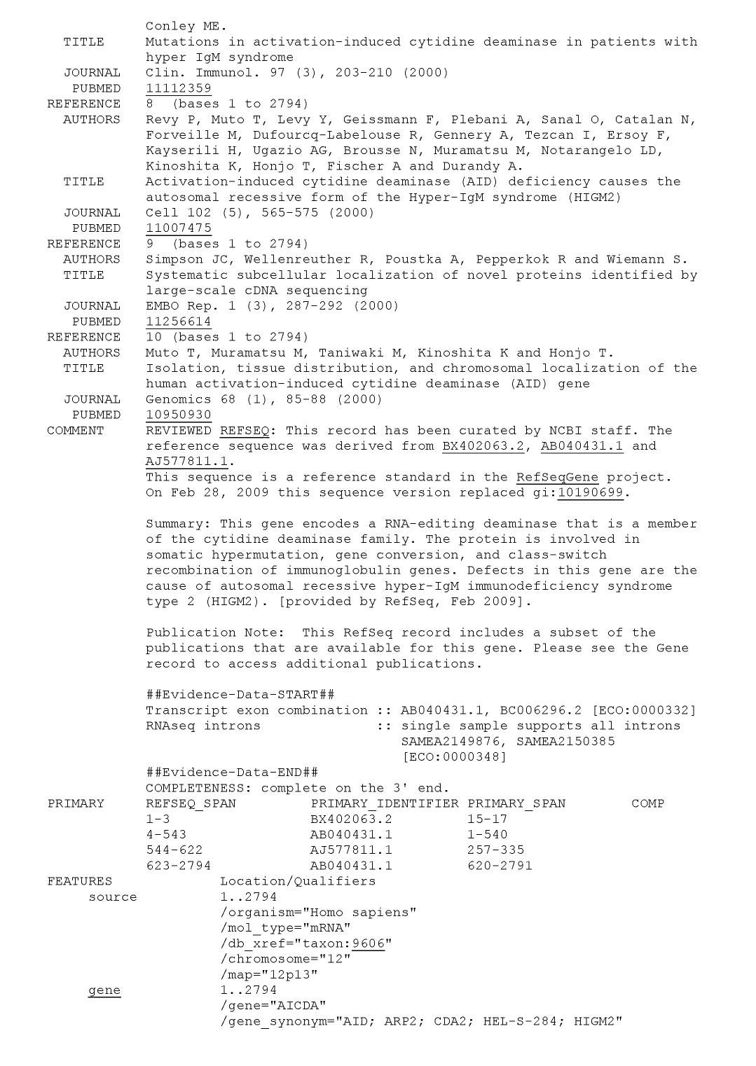

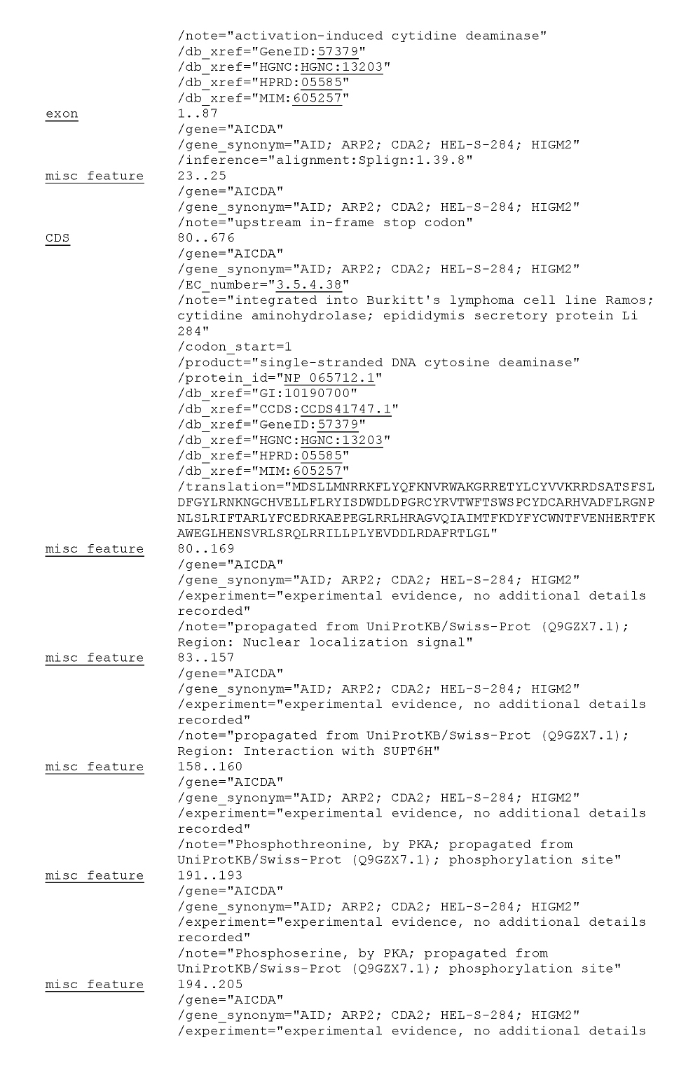

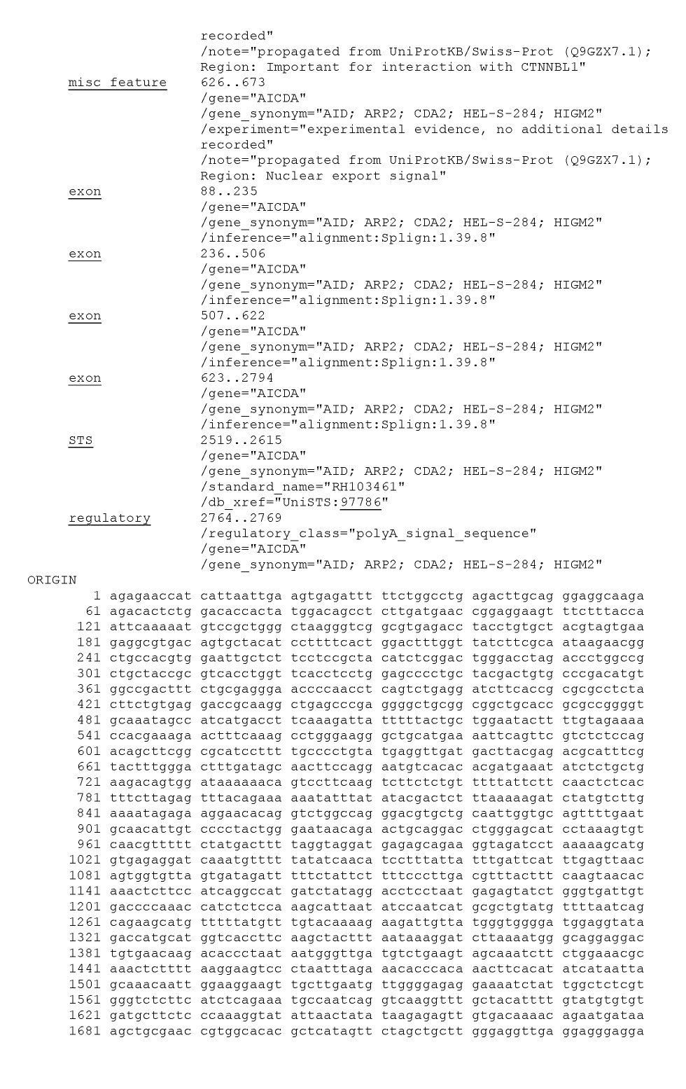

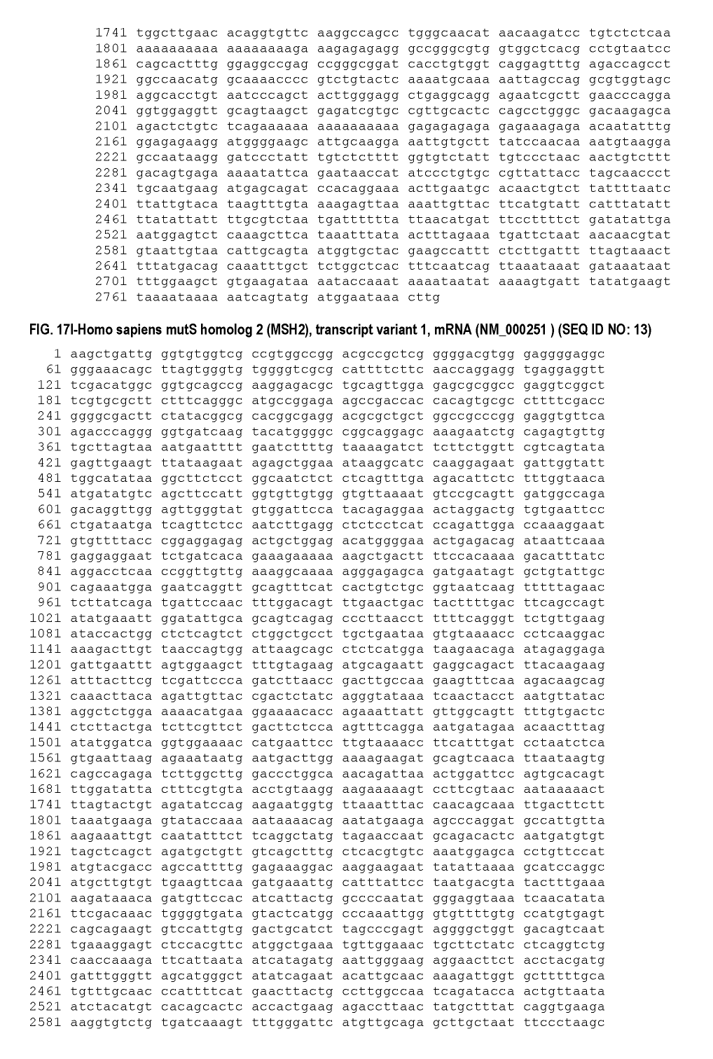

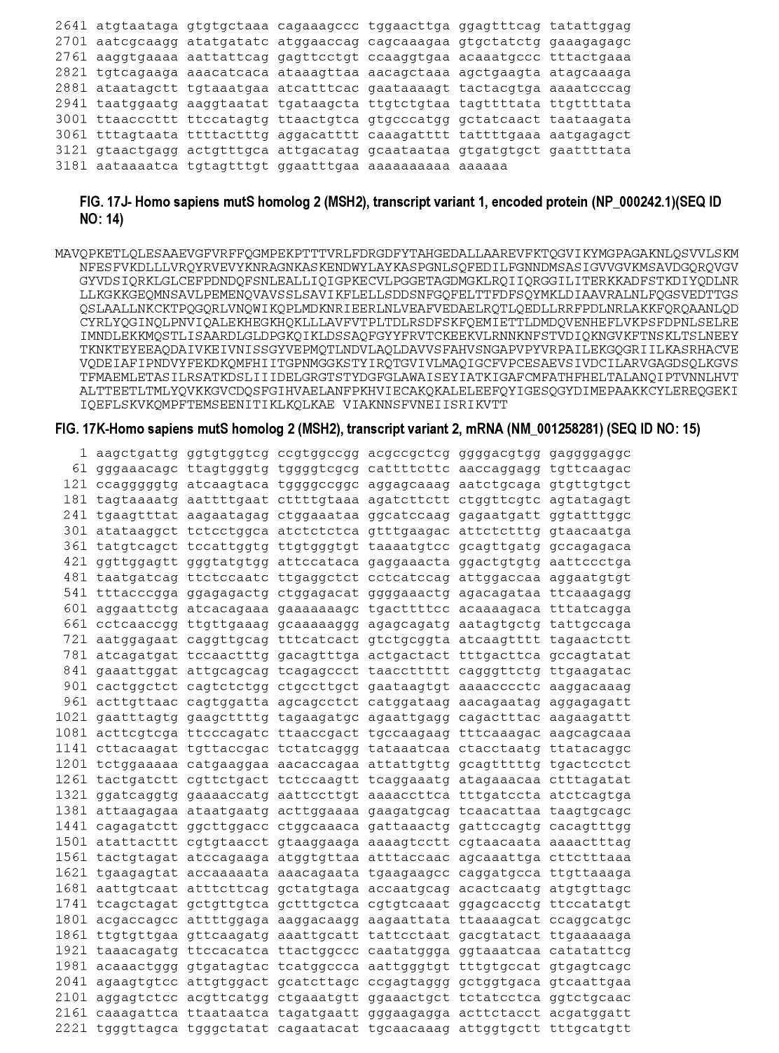

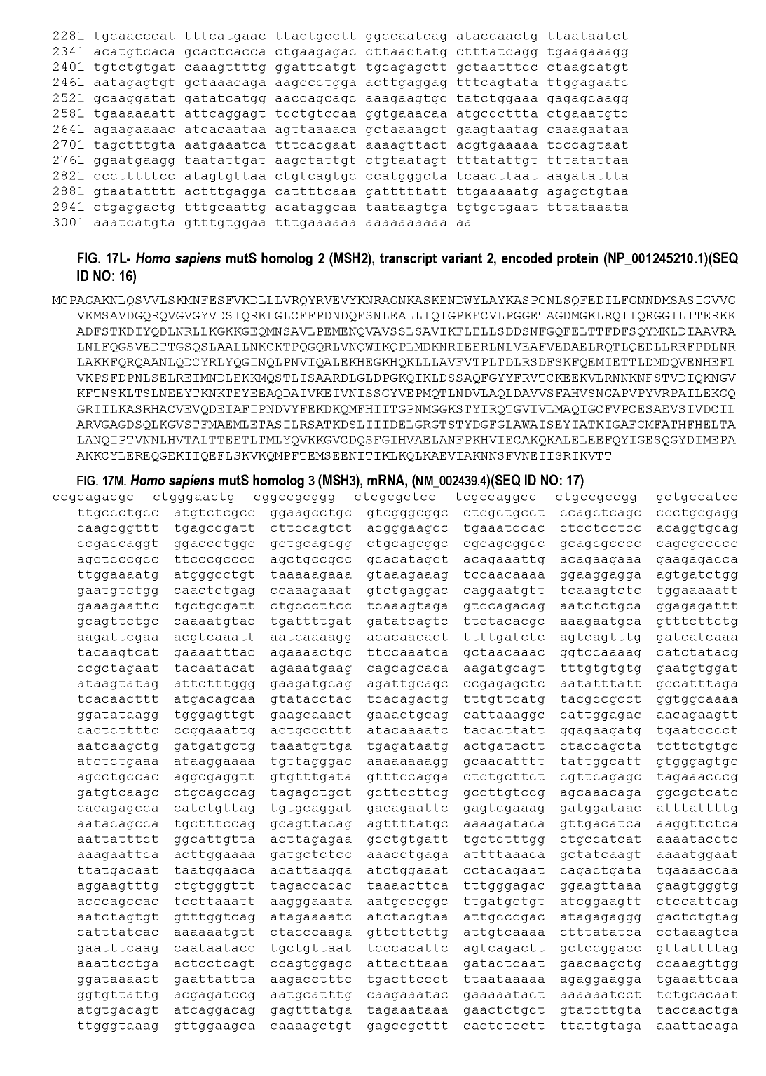

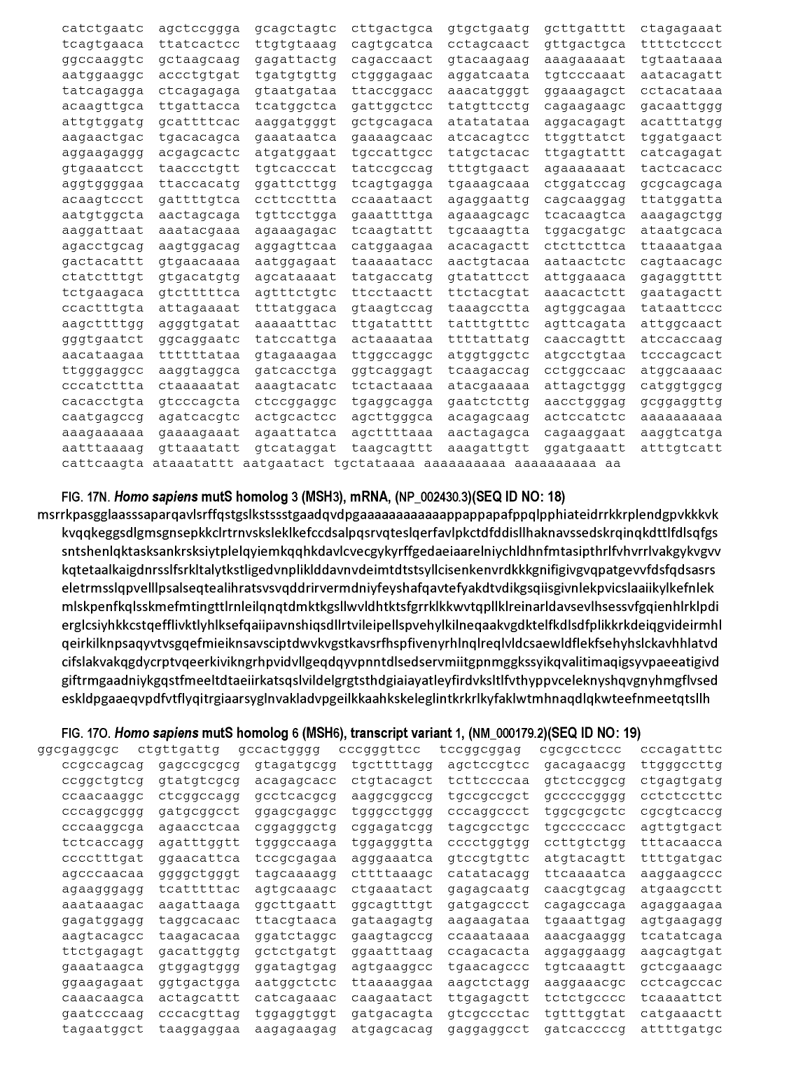

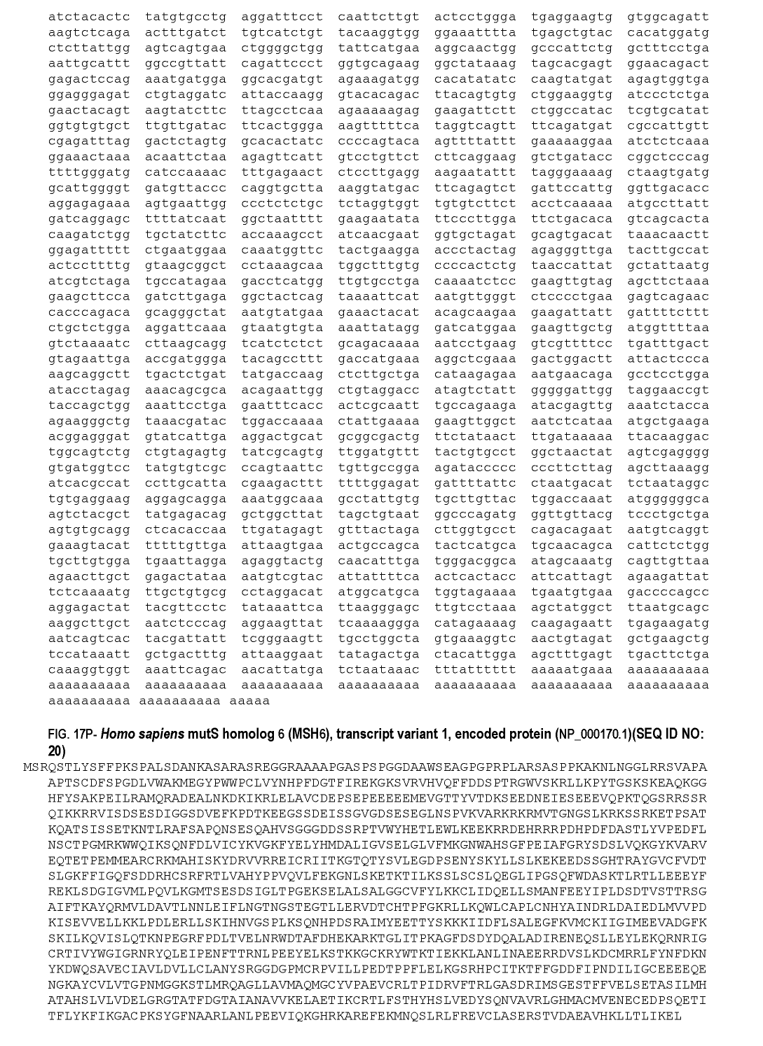

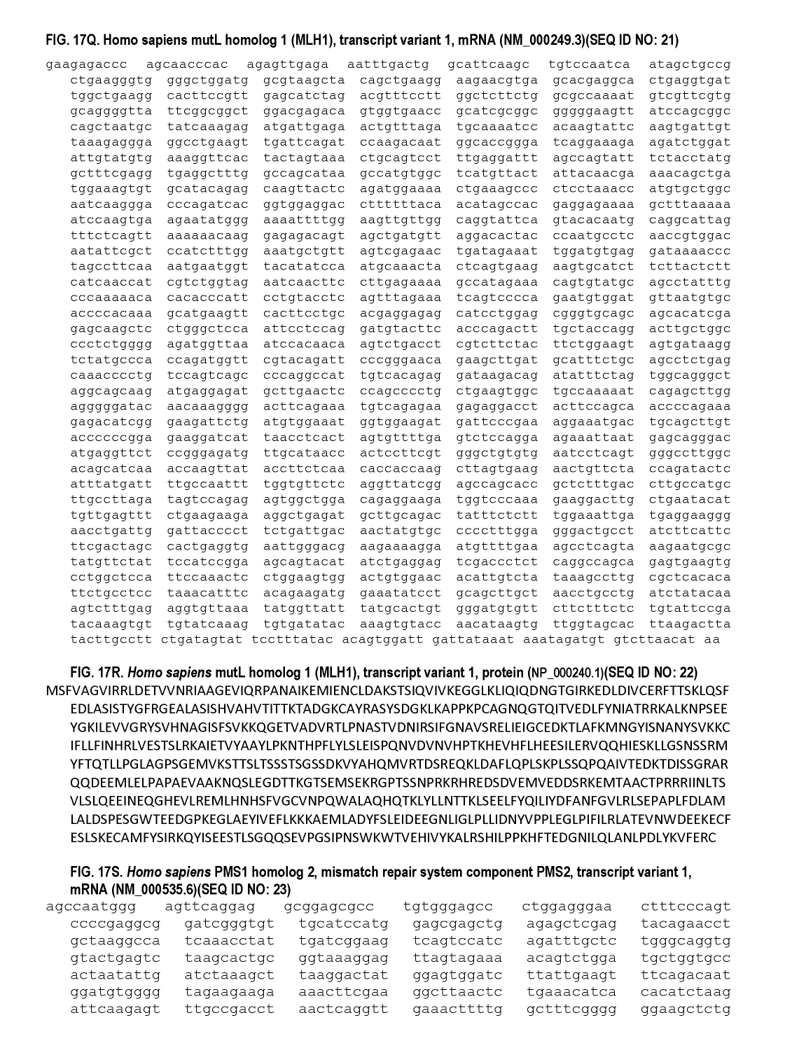

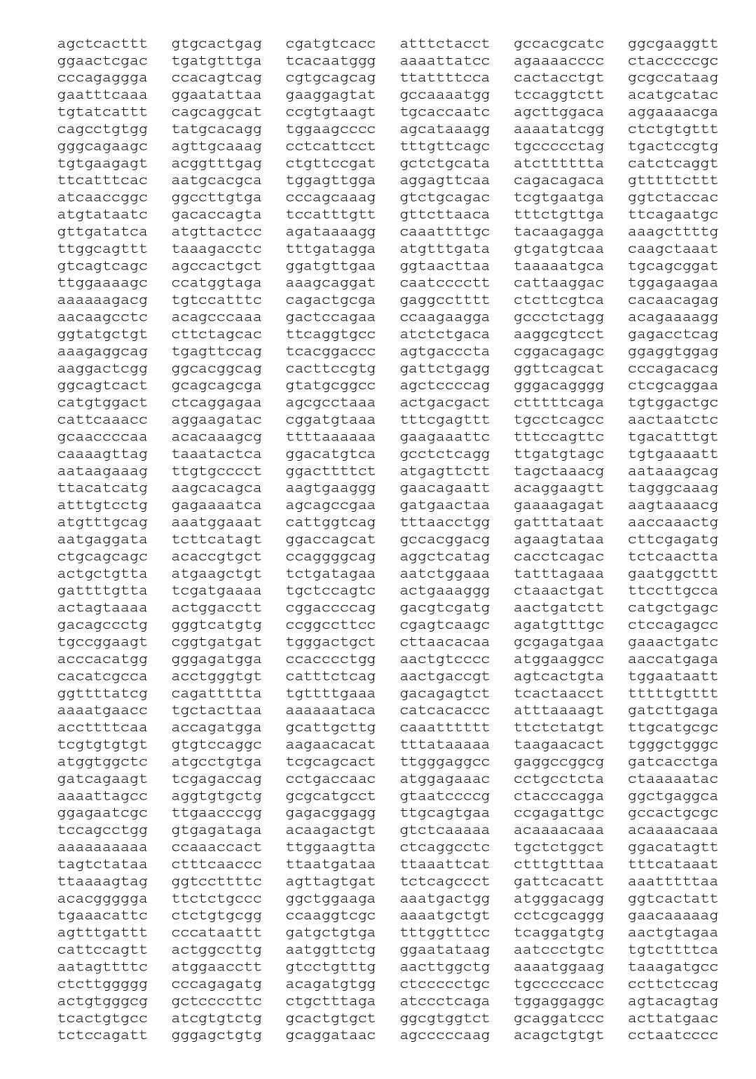

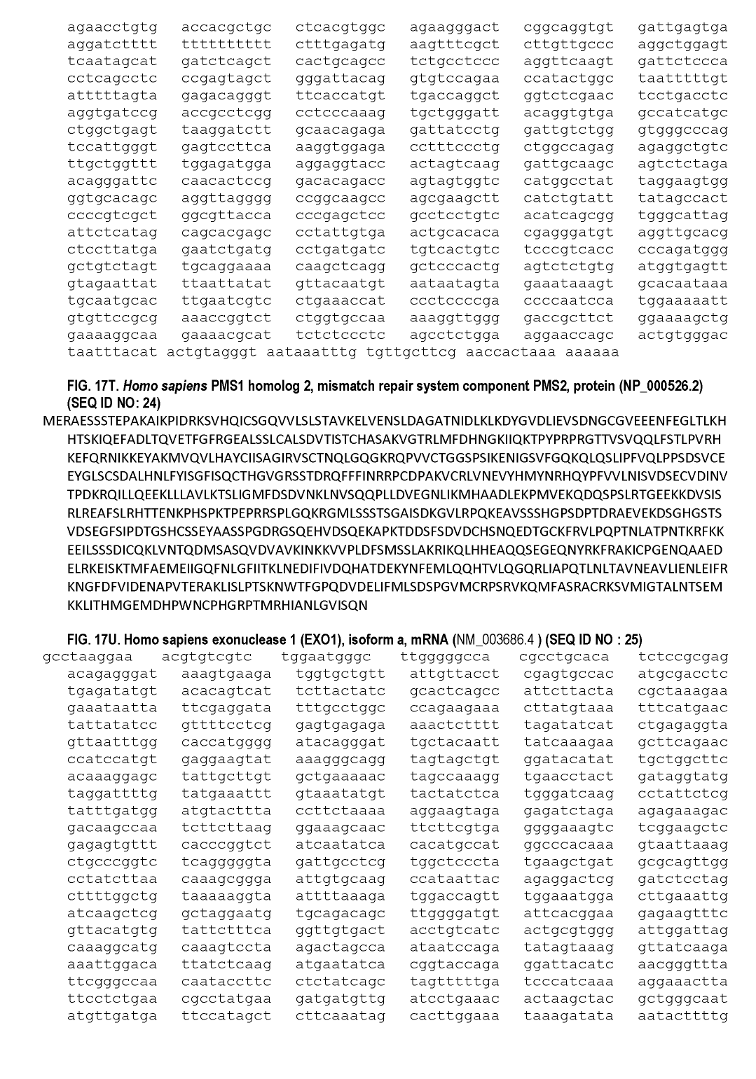

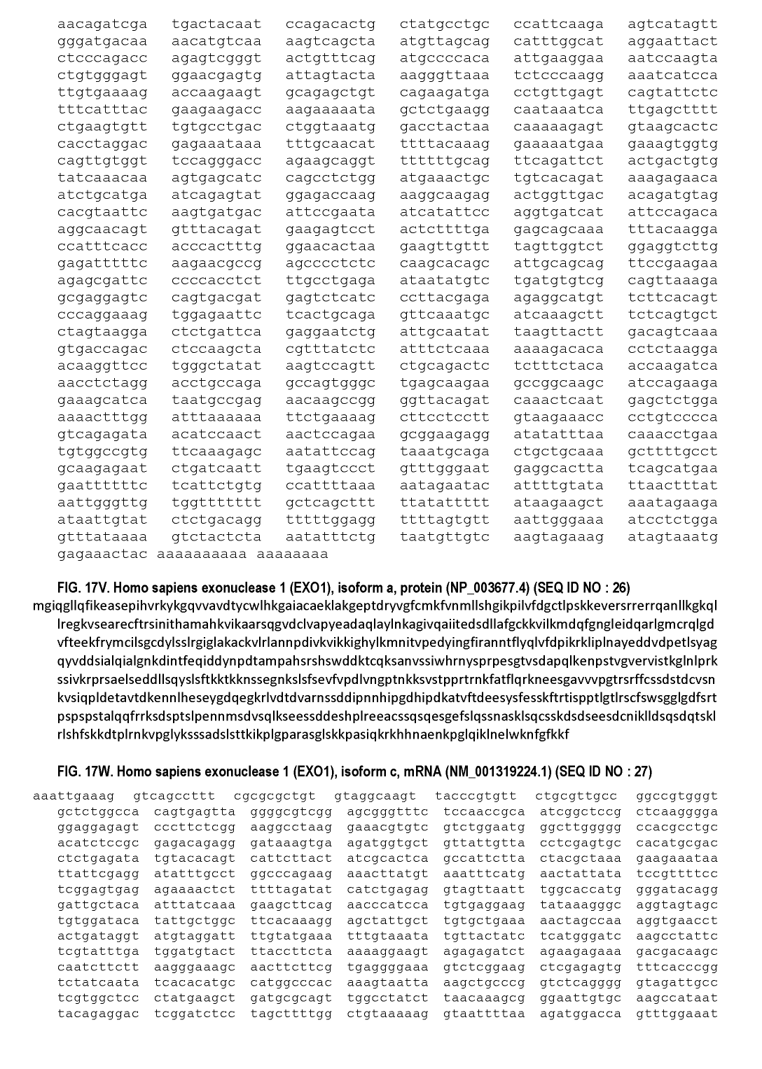

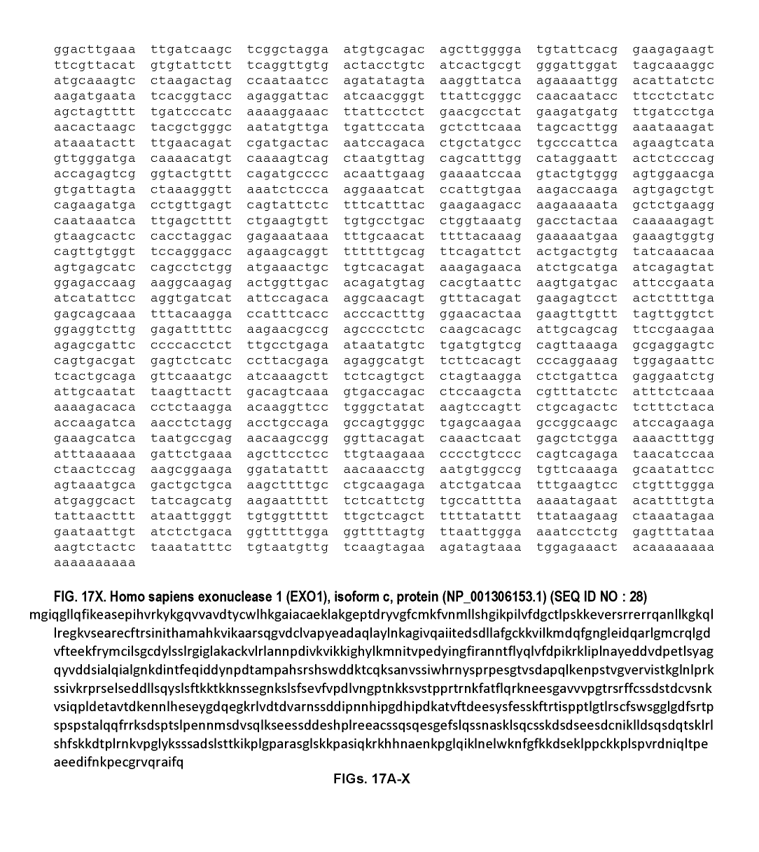

As used herein the terms “UNG gene” refers to nucleic acid (e.g., genomic DNA, cDNA, RNA) encoding the uracil DNA glycosylase (UNG), including UNG1 and/or UNG2, the mitochondrial and nuclear isoforms produced by this gene. The examples described herein used to the UNG2 form. The description of the various aspects and embodiments of the invention is provided with reference to exemplary UNG nucleic acid sequences (SEQ ID NOs: 4 and 6 in FIGS. 17A and C) and amino acid sequence (SEQ ID NOs: 5 and 7 in FIGS. 17B and D). Such reference is meant to be exemplary only and the various aspects and embodiments of the invention are also directed to other UNG nucleic acids and polypeptides (also referred to UNG gene products), such as UNG nucleic acid or polypeptide mutants/variants, splice variants of UNG nucleic acids, UNG variants from species to species or subject to subject.

Consensuses derived from the alignments of certain UNG variants are also presented in FIG. 17E. In specific embodiments of the consensus, each X in the consensus sequence (e.g., consensus in FIG. 17E) is defined as being any amino acid, or absent when this position is absent in one or more of the orthologues (e.g., SEQ ID NOs: 5 and 7) presented in the alignment (SEQ ID NO: 8). In specific embodiment of the consensus, each X in the consensus sequences is defined as being any amino acid that constitutes a conserved or semi-conserved substitution of any of the amino acid in the corresponding position in the orthologues presented in the alignment, or absent when this position is absent in one or more of the orthologues presented in the alignment. In FIG. 17E, conservative substitutions are denoted by the symbol “:” and semi-conservative substitutions are denoted by the symbol “.”. In another embodiment, each X refers to any amino acid belonging to the same class as any of the amino acid residues in the corresponding position in the orthologues presented in the alignment, or absent when this position is absent in one or more of the orthologues presented in the alignment. In another embodiment, each X refers to any amino acid in the corresponding position of the orthologues presented in the alignment, or absent when this position is absent in one or more of the orthologues presented in the alignment. The Table below indicates which amino acid belongs to each amino acid class.

| Class | Name of the amino acids |
|---|---|
| Aliphatic | Glycine, Alanine, Valine, Leucine, Isoleucine |
| Hydroxyl or Sulfur/ | Serine, Cysteine, Selenocysteine, Threonine, |
| Selenium-containing | Methionine |
| Cyclic | Proline |
| Aromatic | Phenylalanine, Tyrosine, Tryptophan |
| Basic | Histidine, Lysine, Arginine |
| Acidic and their | Aspartate, Glutamate, Asparagine, Glutamine |
| Amide | |

MSH2 gene and MSH2 protein

As used herein the terms “MSH2 gene” refers to nucleic acid (e.g., genomic DNA, cDNA, RNA) encoding MSH2. The description of the various aspects and embodiments of the invention is provided with reference to exemplary MSH2 nucleic acid sequences (SEQ ID NOs: 13 and 15 in FIGS. 171 and K) and amino acid sequence (SEQ ID NOs: 14 and 16 in FIGS. 17J and L). Such reference is meant to be exemplary only and the various aspects and embodiments of the invention are also directed to other MSH2 nucleic acids and polypeptides (also referred to MSH2 gene products), such as MSH2 nucleic acid or polypeptide mutants/variants, splice variants of MSH2 nucleic acids, MSH2 variants from species to species or subject to subject.

MMR pathway genes and proteins

As used herein the terms “MMR pathway gene or protein” refer to any gene and encoded protein involved in the MMR pathway. Without being so limited, such genes (e.g., genomic DNA, cDNA, RNA) and proteins include MSH2, MSH3, MSH6, MLH1, PMS2, EXO1, etc. The description of the various aspects and embodiments of the invention is provided with reference to exemplary MSH2 nucleic acid sequences (SEQ ID NOs: 13 and 15 in FIGS. 171 and K) and amino acid sequence (SEQ ID NOs: 14 and 16 in FIGS. 17J and L); MSH3 nucleic acid sequence (SEQ ID NO: 17 in FIG. 17M) and amino acid sequence (SEQ ID NO: 18 in FIG. 17N); MSH6 nucleic acid sequence (SEQ ID NO: 19 in FIG. 170) and amino acid sequence (SEQ ID NO: 20 in FIG. 17P); MLH1 nucleic acid sequence (SEQ ID NO: 21 in FIG. 17Q) and amino acid sequence (SEQ ID NO: 22 in FIG. 17R); PMS2 nucleic acid sequence (SEQ ID NO: 23 in FIG. 17S) and amino acid sequence (SEQ ID NO: 24 in FIG. 17T); EXO1 nucleic acid sequences (SEQ ID NOs: 25 and 27 in FIGS. 17U and W) and amino acid sequences (SEQ ID NOs: 26 and 28 in FIGS. 17V and X);

AID gene and AID protein

As used herein the terms “AID gene” refers to nucleic acid (e.g., genomic DNA, cDNA, RNA) encoding Activation Induced Deaminase (AID) (e.g., sequences comprising those sequences referred to in GenBank by accession number NM_020661 and NG_011588 for the human gene. Although the term AICDA is typically used when designating the gene encoding AID, the expression “AID gene” will sometimes be used herein for convenience and consistency. The description of the various aspects and embodiments of the invention is provided with reference to exemplary AID nucleic acid sequences (SEQ ID NOs: 9, 12) and amino acid sequence (SEQ ID NOs: 10-11) (FIGS. 17f to h). Such reference is meant to be exemplary only and the various aspects and embodiments of the invention are also directed to other AID nucleic acids and polypeptides (also referred to AID gene products), such as AID nucleic acid or polypeptide mutants/variants, splice variants of AID nucleic acids, AID variants from species to species or subject to subject. Without being so limited, those include AID sequences at accession numbers NG_011588 Homo sapiens activation-induced cytidine deaminase (AICDA) on chromosome 12 gi|224994215|ref|NG_011588.1| [224994215; NC_000012 Homo sapiens chromosome 12, GRCh37 primary reference assembly gi|224589803|ref|NC_000012.11||gpp|GPC_000000036.1||gnl|NCBI_{—GENOMES|}12 [224589803; NT_009714 Homo sapiens chromosome 12 genomic contig, GRCh37 reference primary assembly gi|224514867|ref|NT_009714.17||gpp|GPS_000125290.1| [224514867]; NM_020661 Homo sapiens activation-induced cytidine deaminase (AICDA), mRNA gi|224451012|ref|NM_020661.2| [224451012]; 5: AC_000144 Homo sapiens chromosome 12, alternate assembly HuRef, whole genome shotgun sequence gi|157704453|ref|AC_000144.1||gnl|NCBI_{—GENOMES|}21406 [157704453]; NW_001838051 Homo sapiens chromosome 12 genomic contig, alternate assembly (based on HuRef), whole genome shotgun sequence gi|157696928|ref|NW_001838051.1| [157696928]; DQ896237 Synthetic construct Homo sapiens clone IMAGE:100010697; FLH191441.01L; RZPDo839D0467D activation-induced cytidine deaminase (AICDA) gene, encodes complete protein gi|123999319|gb|DQ896237.2| [123999319]; DQ892989 Synthetic construct clone IMAGE:100005619; FLH191445.01X; RZPDo839D0477D activation-induced cytidine deaminase (AICDA) gene, encodes complete protein gi|123990479|gb|DQ892989.2|[123990479]; AM393608 Synthetic construct Homo sapiens clone IMAGE:100002005 for hypothetical protein (AICDA gene)gi|117646033|emb|AM393608.1|[117646033]; DQ431660 Homo sapiens activation-induced cytidine deaminase mRNA, partial cds gi|90200384|gb|DQ431660.1|[90200384]; AC_000055 Homo sapiens chromosome 12, alternate assembly Celera, whole genome shotgun sequence gi|89161189|ref|AC_000055.1||gn|NCBI_{—GENOMES|}18894 [89161189]NW_925295 Homo sapiens chromosome 12 genomic contig, alternate assembly (based on Celera), whole genome shotgun sequence gi|89035948|ref|NW_925295.1| [89035948]; CH471116 Homo sapiens 211000035838052 genomic scaffold, whole genome shotgun sequence gi|74230026|gnl|WGS:AADB|211000035838052|gb|CH471116.2| [74230026]; CS056120 Sequence 39 from Patent W02005023865 gi|62122322|emb|CS056120.1||pat|WO|2005023865|39 [62122322]; AY748364 Homo sapiens activation-induced deaminase (AICDA) mRNA, partial cds gi|53854919|gb|AY748364.1| [53854919]; CR615215 full-length cDNA clone CS0DL012YD18 of B cells (Ramos cell line) Cot 25-normalized of Homo sapiens (human)gi|50496022|emb|CR615215.1| [50496022]; AY541058 Homo sapiens activation-induced cytidine deaminase (AICDA) mRNA, complete cds, alternatively spliced gi|46484694|gb|AY541058.1| [46484694]; AY536517 Homo sapiens activation-induced cytidine deaminase (AICDA) mRNA, complete cds, alternatively spliced gi|46403718|gb|AY536517.1| [46403718]; AY536516 Homo sapiens activation-induced cytidine deaminase (AICDA) mRNA, complete cds, alternatively spliced gi|46403716|gb|AY536516.1| [46403716]; AY534975 Homo sapiens activation-induced cytidine deaminase (AICDA) mRNA, complete cds, alternatively spliced gi|46371948|gb|AY534975.1| [46371948]; BC006296 Homo sapiens activation-induced cytidine deaminase, mRNA (cDNA clone MGC:12911 IMAGE:4054915), complete cds gi|33871601|gb|BC006296.2| [33871601]; AJ577811 Homo sapiens partial mRNA for activation-induced cytidine deaminase (AID gene) gi|33145978|emb|AJ577811.1| [33145978]; BT007402 Homo sapiens activation-induced cytidine deaminase mRNA, complete cds gi|30583642|gnl|clontech|GH00009X1.0|gb|BT007402.1| [30583642]; AB092577 Homo sapiens AID gene for activation-induced cytidine deaminase, partial cds, exon 2 gi|29126042|dbj|AB092577.1| [29126042]; AF529827 Homo sapiens clone Ramos 13 AID (AID) mRNA, partial cds gi|22297241|gb|AF529827.1| [22297241]; AF529826 Homo sapiens clone Ramos 12 AID (AID) mRNA, partial cds gi|22297239|gb|AF529826.1| [22297239]; AF529825 Homo sapiens clone Ramos 11 AID (AID) mRNA, partial cds gi|22297237|gb|AF529825.1| [22297237]; AF529824 Homo sapiens clone Ramos 10 AID (AID) mRNA, partial cds gi|22297235|gb|AF529824.1| [22297235]; AF529823 Homo sapiens clone Ramos 9 AID (AID) mRNA, partial cds gi|22297233|gb|AF529823.1| [22297233]; AF529822 Homo sapiens clone Ramos 8 AID (AID) mRNA, partial cds gi|22297231|gb|AF529822.1| [22297231]; AF529821 Homo sapiens clone Ramos 7 AID (AID) mRNA, partial cds gi|22297229|gb|AF529821.1| [22297229]; AF529820 Homo sapiens clone Ramos 6 AID (AID) mRNA, partial cds gi|22297227|gb|AF529820.1| [22297227]; AF529819 Homo sapiens clone Ramos 5 AID (AID) mRNA, partial cds gi|22297225|gb|AF529819.1| [22297225]; AF529818 Homo sapiens clone Ramos 4 truncated AID (AID) mRNA, complete cds gi|22297223|gb|AF529818.1| [22297223]; AF529817 Homo sapiens clone Ramos 3 AID (AID) mRNA, partial cds gi|22297221|gb|AF529817.1| [22297221]; AF529816 Homo sapiens clone Ramos 2 AID (AID) mRNA, partial cds gi|22297219|gb|AF529816.1| [22297219]; AF529815 Homo sapiens clone Ramos 1 AID (AID) mRNA, partial cds gi|22297217|gb|AF529815.1| [22297217]; AC092184 Homo sapiens 12 BAC RP11-438L7 (Roswell Park Cancer Institute Human BAC Library) complete sequence gi|21206067|gnl|bcmhgsc|project_hdkj.baylor|gb|AC092184.7| [21206067]; AB040431 Homo sapiens AID mRNA for activation-induced cytidine deaminase, complete CDS gi|9988409|dbj|AB040431.1| [9988409]; AB040430 Homo sapiens AID gene for activation-induced cytidine deaminase, complete cds gi|9988407|dbj|AB040430.1| [9988407]. Without being so limited, examples of mutant variants are described in ^{62,63}.

Proteins detected in accordance with the present invention (e.g., AID, UNG, MMR proteins (MSH2, MSH3, MSH6, MLH1, PMS2, EXO1) may be as depicted in FIGS. 17A-X or may be variants thereof. Hence proteins detected in accordance with the present invention include proteins with amino acid sequences having high percent identities (e.g., at least 50%, 55%, 60%, 65%, 70%, 75%, 80%, 85%, 90%, 95%, 96%, 97%, 97%, 98% and 99% identity) with proteins specifically disclosed in FIGS. 17A to X.

UNG expression or AID expression or MMR expression

As used herein the terms “AID expression level” or “AID expression”, or “UNG expression level” or “UNG expression” or “MMR expression level” or “MMR expression”, refer to the measurement in a cell or a tissue of an AID or UNG or MMR gene product, respectively. AID and UNG and MMR expression levels could be evaluated at the polypeptide and/or nucleic acid levels (e.g., DNA or RNA) using any standard methods known in the art. The nucleic acid sequence of a nucleic acid molecule in a sample can be detected by any suitable method or technique of measuring or detecting gene sequence or expression. Such methods include, but are not limited to, polymerase chain reaction (PCR), reverse transcriptase-PCR (RT-PCR), in situ PCR, SAGE, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

In the context of this invention, “hybridization” means hydrogen bonding between complementary nucleoside or nucleotide bases. Terms “specifically hybridizable” and “complementary” are the terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. Such conditions may comprise, for example, 400 mM NaCl, 40 mM PIPES pH 6.4, 1 mM EDTA, at 50 to 70oC for 12 to 16 hours, followed by washing. The skilled person will be able to determine the set of conditions most appropriate for a test of complementarity of two sequences in accordance with the ultimate application of the hybridized nucleotides.

Methods to measure protein expression levels of selected genes of this invention, include, but are not limited to: Western blot, tissue microarray, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), flow cytometry, and assays based on a property of the protein including but not limited to DNA binding, ligand binding, or interaction with other protein partners. In a further embodiment, the AID and/or UNG expression level is measured by immunohistochemical staining, and the percentage and/or the intensity of immunostaining of immunoreactive cells in the sample is determined.

In an embodiment, the level of an UNG and/or AID and/or MMR (e.g., MSH2, MSH3, MSH6, MLH1, PMS2,EXO1) polypeptide is determined using an anti-UNG or anti-AID or an anti-MMR antibody. By “UNG antibody” and “anti-UNG” or “AID antibody” and “anti-AID” or “MMR antibody” and “anti-MMR” (e.g., anti-MSH2, anti-MSH3, anti-MSH6, anti-MLH1, anti-PMS2, anti-EXO1), in the present context is meant an antibody capable of detecting (i.e. binding to) an UNG protein or an UNG protein fragment or an AID protein or an AID protein fragment or an MMR protein or an MMR protein fragment, respectively. Without being limited, UNG antibodies include those listed in Table I below, AID antibodies include those listed in Table II below, and MMR antibodies include those listed in Table III below.

TABLE I
Examples of commercially available UNG antibodies

| Company | Catalog number | Name |
|---|---|---|
| Abcam | ab23926 | Rabbit polyclonal |
| | ab109214 | Rabbit monoclonal |
| | ab62520 | Rabbit polyclonal |
| | ab47680 | Rabbit polyclonal |
| | ab189401 | Rabbit polyclonal |
| | ab89640 | Rabbit polyclonal |
| | ab118977 | Mouse monoclonal |
| | ab58976 | Mouse monoclonal |
| | ab101283 | Rabbit polyclonal |
| | ab101283 | Rabbit polyclonal |

TABLE II
Examples of commercially available AID antibodies

| Company | Catalog number | Name |
|---|---|---|
| Cell signaling technologies | 4959 | EK2 5G9 Rat mAb |
| | 4975 | L7E7 Mouse mAb |
| | 30F12 | Rabbit mAb |
| Abcam | Ab5197 | Rabbit polyclonal |
| | Ab59361 | Rabbit polyclonal |
| | Ab77401 | Goat polyclonal |
| | Ab56147 | Rabbit polyclonal |
| Genway | 18-202-336474 | Rabbit polyclonal |
| | 18-783-313040 | Rabbit polyclonal |
| Active Motif | 39885 | Rat monoclonal |

TABLE III
Examples of commercially available MMR antibodies

| | | |
|---|---|---|
| Company | Catalog number | Name |

MSH2

| | | |
|---|---|---|
| Abcam | sc-376501 | Mouse monoclonal |
| | sc-515356 | Mouse monoclonal |
| | sc-494 | Polyclonal |
| Santa Cruz Biotech | sc-22771 | |

MSH3

| | | |
|---|---|---|
| Abcam | Ab61619 | Rabbit polyclonal |
| | ab111107 | Rabbit polyclonal |

MSH6

| | | |
|---|---|---|
| Abcam | ab92471 | Rabbit monoclonal |
| | ab10340 | Goat polyclonal |
| Cell signaling | 3995 | Rabbit polyclonal |

MHL1

| | | |
|---|---|---|
| Abcam | ab9144 | Rabbit polyclonal |
| Cell signaling | 3515 | Mouse monoclonal |
| Miltenyi | 130-109-408 | Human monoclonal |
| | 130-109-409 | Human monoclonal |
| | 130-109-410 | Human monoclonal |
| | 130-109-411 | Human monoclonal |

PMS2

| | | |
|---|---|---|
| Aviva system | ARP56117_P050 | Rabbit polyclonal |
| | OAAF04086 | Rabbit polyclonal |
| | OAAF03066 | Rabbit polyclonal |
| | OARA02371 | Mouse monoclonal |

EXO1

| | | |
|---|---|---|
| | sc-19941 | goat polyclonal |
| | sc-33194 | rabbit polyclonal |
| | sc-56092 | Mouse polyclonal |
| | sc-56387 | mouse polyclonal |
| Novus Biologicals | NBP1-19709 | |

Methods for normalizing the level of expression of a gene are well known in the art. For example, the expression level of a gene of the present invention can be normalized on the basis of the relative ratio of the mRNA level of this gene to the mRNA level of a housekeeping gene, or the relative ratio of the protein level of the protein encoded by this gene to the protein level of the housekeeping protein, so that variations in the sample extraction efficiency among cells or tissues are reduced in the evaluation of the gene expression level. A “housekeeping gene” is a gene the expression of which is substantially the same from sample to sample or from tissue to tissue, or one that is relatively refractory to change in response to external stimuli. A housekeeping gene can be any RNA molecule other than that encoded by the gene of interest that will allow normalization of sample RNA or any other marker that can be used to normalize for the amount of total RNA added to each reaction. For example, the GAPDH gene, the G6PD gene, the actin gene, ribosomal RNA, 36B4 RNA, PGK1, RPLP0, or the like, may be used as a housekeeping gene.

Methods for calibrating the level of expression of a gene are well known in the art. For example, the expression of a gene can be calibrated using reference samples, which are commercially available. Examples of reference samples include, but are not limited to: Stratagene™ QPCR Human Reference Total RNA, Clontech™ Universal Reference Total RNA, and XpressRef™ Universal Reference Total RNA.

In an embodiment, the above-mentioned method comprises determining the level of an AID and/or UNG and/or MMR (MSH2, MSH3, MSH6, MLH1, PMS2 and EX01) nucleic acid (e.g., nucleic acids as shown in FIGS. 17A-X) in the sample. In another embodiment, the above-mentioned method comprises determining the level of an AID and/or UNG and/or MMR (MSH2, MSH3, MSH6, MLH1, PMS2 and EXO1) polypeptide (e.g., polypeptides as shown in FIGS. 17A-X) in the sample.

UNG activity

As used herein the terms “UNG activity” and “UNG function” are used interchangeably and refer to detectable (direct or indirect) enzymatic (e.g., base excision repair (BER) i.e. uracil excision to create an abasic site), biochemical or cellular activity attributable to UNG. Without being so limited, such activities include uracil excision from single- and/or double-stranded DNA; removing genomic uracil arising from dUMP misincorporation during replication, removing genomic uracil arising from spontaneous or enzymatic deamination of dC, initiating error-prone DNA repair pathways leading to somatic hypermutation and class switch recombination at the immunoglobulin genes, preventing mutations arising from replication over genomic uracil, and removing uracil generated by AID-catalyzed deamination of dC at the C-rich strand of the telomeres and/or elsewhere in the genome. UNG activity could also be indirectly measured by evaluating the level of expression of UNG, or a fragment thereof, in cells as well as in biological samples (e.g., tissue, organ, fluid).

AID activity

As used herein the terms “AID activity” and “AID function” are used interchangeably and refer to detectable (direct or indirect) enzymatic (e.g., deamination of deoxycytidine (dC) to deoxyuridine (dU) for example in telomeric C-rich strand), biochemical or cellular activity attributable to AID. Without being so limited, such activities include deamination of dC to dU for example in telomeric C-rich strand, the binding of AID to CHIP, the effect of AID on cellular genomic plasticity such as a dU-induced DNA break, a DNA translocation, a DNA deletion, a DNA recombination (including region-specific recombination between isotype switch regions, immunoglobulin gene conversion, homologous recombination) or a general or localized mutator effect. Other activities of AID include Ig gene (i.e. encoding antibody) diversification by somatic hypermutation (SHM) and class switch recombination (CSR) (e.g., IgM to IgG, IgE or IgA). In a specific embodiment, AID activity refers to deamination of deoxycytidine (dC) to deoxyuridine (dU) in telomeric C-rich strand. Assays measuring SHM and CSR are described in Example 1 of US-2011-0237560-A1 and results of these assays are presented in Examples 5 and 6 of US-2011-0237560-A1 for example. AID activity could also be indirectly measured by evaluating the level of expression of AID, or a fragment thereof, in cells as well as in biological samples (e.g., tissue, organ, fluid).

MMR pathway activity

As used herein the terms “MMR pathway activity” refer to detectable (direct or indirect) enzymatic (e.g., initiation of the formation of DNA double stranded breaks), biochemical or cellular activity attributable to proteins involved in the MMR pathway such as MSH2, MSH3, MSH6, MLH1, PMS2 and EXO1.

As used therein, a “functional MMR pathway” is meant to refer to a MMR pathway able to perform its activities leading to DNA double stranded breaks i.e. detect G:U mismatches and initiate the formation of DNA double stranded breaks. Without being so limited, a functional MMR pathway expresses active MSH2, MSH3, MSH6, MLH1, PMS2 and EXO1 or a combination of at least two, at least three, at least four, at least 5 or at least 6 thereof) and/or MSH2/MSH6 heterodimer activity and/or MSH2/MSH3 heterodimer activity and/or MLH1/PMS2 heterodimer activity and/or MLH1/PMS2 heterodimer activity and/or EXO1.

Modulation of UNG and/or AID expression or activity

The modulation of UNG expression and/or activity or AID expression and/or activity could be achieved directly or indirectly by various mechanisms, which among others could act at the level of (i) transcription, for example by inhibiting the UNG promoter reducing the UNG messenger RNA expression or AID promoter increasing the AID messenger RNA expression (e.g., by cytokine stimulation, Toll-like receptor stimulation, estrogen-estrogen receptor complex, HCV core protein, EBV LMP2, etc.), (ii) translation, (iii) post-translational modifications, e.g., glycosylation, sulfation, phosphorylation, ubiquitination (e.g., polyubiquitinylation and proteasomal degradation), (iv) cellular localization (e.g., cytoplasmic versus nuclear localization), (v) protein-protein interaction, for example for AID, by modulating expression and/or activity of a protein that binds to and stabilizes AID and/or limits its nuclear access (e.g., eukaryotic elongation factor 1 α (eEF1A)). These regulatory processes occur through different molecular interactions that could be modulated using a variety of compounds or modulators.

An important step regulating AID is subcellular localization. Most of the enzyme is in the cytoplasm in steady state, which is determined by the integration of three mechanisms: nuclear import, nuclear export and cytoplasmic retention. The compartmentalization of AID determines its stability: AID is destabilized in the nucleus by polyubiquitinylation and proteasomal degradation.

In accordance with the present invention, modulation of AID expression and/or activity encompasses favoring AID nuclear localization pursuant to results reported in Methot et al. ⁶⁶, incorporated herein by reference, which show that compounds that inhibit the translation elongation factor eukaryotic elongation factor 1 α (eEF1A), an enzyme that retains AID in the cytoplasm, favors AID nuclear accumulation/localization which in turn increases AID's mutator activity on normal and neoplastic cells. eEF1A inhibitors include didemnin B (DidB) and cytotrienin A (CytA)).

As indicated above, modulation of AID mutator activity can also be achieved by the activity resulting from genes known to control/prevent/repair AID-mediated mutations and/or AID-mediated antibody diversification. These include, amongst others, MMR, p53, ATM, Nbs1, p19(Arf), PUMA, Bim, PKC-delta and UNG2.

In the context of the present invention, a “compound” is a molecule such as, without being so limited, an siRNA, antisense molecule, protein, peptide, small molecule, antibody, etc.

UNG inhibitors

As used herein, “UNG inhibitor” refers to any compound or composition that directly or indirectly inhibits UNG's expression and/or activity. Without being so limited, candidate compounds modulating the UNG expression and/or activity are tested using a variety of methods and assays. It includes molecules such as, without being so limited, siRNA, antisense molecule, protein, peptide, small molecule, antibody, etc.

As used herein, “inhibition” or “decrease” of UNG expression and/or activity refers to a reduction in UNG expression level or activity level of at least 5% as compared to reference UNG expression and/or activity (e.g., a measurement of UNG expression and/or activity in the subject before treatment with an UNG inhibitor). In an embodiment, the reduction in UNG expression level or activity level is of at least 10% lower, in a further embodiment, at least 15% lower, in a further embodiment, at least 20% lower, in a further embodiment of at least 30%, in a further embodiment of at least 40%, in a further embodiment of at least 50% lower, in a further embodiment of at least 60% lower, in a further embodiment of at least 70% lower, in a further embodiment of at least 80%, in a further embodiment of at least 90%, in a further embodiment of 100% (complete inhibition).

Preferably, an UNG inhibitor is a compound having a low level of cellular toxicity and acting in a reversible manner.

Peptides-based UNG inhibitors include the Uracil Glycosylase Inhibitor (UGI) of Bacillus subtilis bacteriophage PBS1. It is a small protein (9.5 kDa) which inhibits E. coli UNG as well as UNG from other species (e.g., New England BioLabs catalog No. M0281S) Ugi Seq. ID P14739.1.

(SEQ ID NO: 29)
(>gi|215789|gb|AAAg1582.1| uracil-DNA glycosylase
inhibitor [Bacillus phage PBS2]
MTNLSDIIEKETGKQLVIQESILMLPEEVEEVIGNKPESDILVHTAYDE
STDENVMLLTSDAPEYKPWALVIQDSNGENKIKML).

Inhibition of UNG occurs by reversible protein binding with a 1:1 UNG:UGI stoichiometry.

Crystallographic structures of Ugi and UNG complexes, including human UNG, are available and can be used for in silico design of small molecule inhibitors (Acharya et al.⁶⁵).

UNG small molecule inhibitors are described in Krosky et al.⁶⁴ which is hereby incorporated by reference. Examples of UNG inhibitors are also described in WO2006135763 which is hereby incorporated by reference. This publication described novel compounds providing an inhibitory action against the enzymes of the uracil base excision repair (UBER) pathway, which includes the enzymes UNG, pUTPase and AP endonuclease I. The inhibitor compounds of the invention can comprise an active-site targeting element, a second binding element, and a linker that links the active-site targeting element and the second binding element as described in WO2006135763. The active site targeting element can be a uracil substrate fragment. In one aspect, the compounds can include a third binding element.

Mismatch repair pathway stimulators

As used herein, “Mismatch repair pathway stimulators” or “MMR stimulator” refer to any compound or composition that directly or indirectly increases the heterodimer MSH2/MSH6 and/or MLH1/PMS2 and/or MSH2/MSH3 and/or EXO1's expression and/or activity resulting in an overall genomic DNA mismatch repair increase. Without being so limited, compounds modulating the MSH2/MSH6, MLH1/PMS2 and/or EXO1 expression and/or activity are encompassed by this definition. It includes molecules such as, without being so limited, siRNA, antisense molecule, protein, peptide, small molecule, antibodies, etc. Candidate compounds are tested using a variety of methods and assays.

As used herein, “increase” of MSH2 expression and/or activity refers to an increase in MSH2 expression level or activity level of at least 5% as compared to reference MSH2 expression and/or activity (e.g., a measurement of MSH2 expression and/or activity in the subject before treatment with a MSH2 stimulator). In an embodiment, the increase in MSH2 expression level or activity level is of at least 10% higher, in a further embodiment, at least 15% higher, in a further embodiment, at least 20% higher, in a further embodiment of at least 30% higher, in a further embodiment of at least 40% higher, in a further embodiment of at least 50% higher, in a further embodiment of at least 60% higher, in a further embodiment of at least 70% higher, in a further embodiment of at least 80% lower, in a further embodiment of at least 90% lower, in a further embodiment of 100% lower.

Results reported herein at e.g., Examples 6 and 8, show that MSH2's presence in UNG deficient AID expressing cells, increases telomeric damage and neoplastic cells proliferation damage.

Without being so limited, mismatch repair pathway stimulators includes a MSH2 gene, RNA or protein such as that shown in FIGS. 17I-L(SEQ ID NOs: 13-16), compounds or genes that produce chromatin modifications (including but not limited to acetylation, methylation, phosphorylation, ubiquitylation or combinations thereof) that enhance the recruitment and/or activity of any MMR pathway protein (e.g., inhibitor of HDAC6 (a histone deacetylase), which was shown to increase MSH2/MSH6 levels ⁶⁷), a MSH3 gene, RNA or protein such as that shown in FIGS. 17M-N(SEQ ID NOs: 17-18) a MSH6 gene, RNA or protein such as that shown in FIGS. 17O-P (SEQ ID NOs: 19-20), MLH1 gene, RNA or protein such as that shown in FIGS. 17Q-R (SEQ ID NOs: 21-22), PMS2 gene, RNA or protein such as that shown in FIGS. 17S-T (SEQ ID NOs: 23-24) or EXO1 Gene, RNA or protein such as that shown in FIGS. 17U-X (SEQ ID NOs: 25-28), etc.

AID-associated diseases

As used herein the terminology “AID-associated diseases” refers to AID-expressing diseases proficient for mismatch repair. As used herein “AID-expressing diseases proficient for mismatch repair” refers to diseases, the pathogenic cells of which display AID and a functional mismatch repair pathway, including a functional MSH2/MSH6 heterodimer activity and/or MSH2/MSH3 heterodimer activity and/or MLH1/PMS2 heterodimer activity and/or MLH1/PMS2 heterodimer activity and/or EXO1 and/or an equivalent or functionally redundant exonuclease activity. Without being so limited, such diseases include AID-expressing neoplastic diseases proficient for mismatch repair including AID-expressing solid tumors (e.g., inflammation-associated cancers) and AID-expressing immune system-derived cancers, and other immune system diseases proficient for mismatch repair including atopic allergies and B cell-related autoimmune diseases (e.g., systemic lupus erythematosus).

Among AID-associated diseases certain are estrogen-driven (e.g., caused by treatment with estrogen) including certain AID-expressing neoplastic diseases such as certain breast and ovarian cancer, and certain B cell-related autoimmune diseases such as Rheumatoid Arthritis, System Lupus Erythematosus and Multiple Sclerosis.

“AID-expressing immune system-derived cancers” include herein but are not restricted to, chronic myeloid leukemia (CML) ²³; acute lymphoblastic leukemia (e.g., BCR-ABL1-positive ALL) ²²; human B cell non-Hodgkin's lymphomas (B-NHLs), such as follicular lymphoma (FL) ^{19,20,33,69}, Burkitt lymphoma ^{19,20}, all subtypes of diffuse large B-cell lymphoma (DLBCL) ^{19,20,44,69} and AIDS-associated B-NHL ⁵⁴ as well as in B-cell chronic lymphocytic leukemia (B-CLL), and its tissue counterpart, small lymphocytic lymphoma (SLL) ^{33,70}.

“AID-expressing solid tumors” include herein but are not restricted to, stomach tumor (e.g., Helicobacter pylori infection-associated stomach tumor), gastric adenocarcinomas ⁶⁸, cholangiocarcinoma ³², lymph node lymphomas ^{19,20,44,69}, lung tumor ¹⁸, liver tumor ⁷¹, colitis-associated colorectal cancers ²⁴, brain tumor, ovary tumor (e.g., ovary carcinoma, endometriosis or adenocarcinoma), breast tumor ⁷² (e.g., breast fibroadenoma or carcinoma), skin tumor (e.g., skin melanoma), prostate carcinoma, bladder tumor (e.g., bladder adenocarcinoma), vascular endothelium hemangioma, kidney carcinoma, thyroid follicular adenoma, relapsed-refractory multiple myeloma. Several data strongly suggest the involvement of AID in inflammation-associated carcinogenesis in humans ³⁴. For instance, aberrant AID expression was revealed in colonic mucosa and cancer tissues of patient with inflammatory bowel disease, but not in normal colonic mucosa.

In one embodiment, the present invention relates to benign neoplastic disease. In another embodiment the present invention relates to malignant neoplastic disease. In specific embodiments, the malignant neoplastic disease is cancer.

In an embodiment, the above-mentioned cancer/tumor is associated with AID expression and/or activity (e.g., aberrant or increased AID expression and/or activity, also referred to as AID-expressing or AID-positive tumor). In one embodiment, the above-mentioned cancer is a cancer of the immune system.

In another embodiment, the above-mentioned cancer/tumor is a solid tumor.

Treatment and prevention

The terms “treat/treating/treatment” and “prevent/preventing/prevention” as used herein, refers to eliciting the desired biological response, i.e., a therapeutic and prophylactic effect, respectively. In accordance with the subject invention, the therapeutic effect comprises one or more of a decrease/reduction in the severity of a human disease (e.g., a reduction or inhibition of cancer progression and/or metastasis development or reduction or inhibition of an autoimmune disease), a decrease/reduction in at least one symptom or disease-related effect, an amelioration of at least one symptom or disease-related effect, a decrease/reduction of the development of the cancer resistance to a drug treatment, and an increased survival time of the affected host animal, following administration of the at least one UNG inhibitor (or of a composition comprising the inhibitor). In accordance with the invention, a prophylactic effect may comprise a complete or partial avoidance/inhibition of cancer or a delay of cancer (e.g., a complete or partial avoidance/inhibition of metastasis development or a delay of metastasis development), of drug resistance, or of autoimmune disease development/progression, and an increased survival time of the affected host animal, following administration of the at least one UNG inhibitor or of a composition comprising the inhibitor).

As such, a “therapeutically effective” or “prophylactically effective” amount of UNG inhibitor affecting UNG expression and/or activity, or a combination of such inhibitors, may be administered to an animal, in the context of the methods of treatment and prevention, respectively, described herein.

Types of samples from the subject and of control samples

As used herein, the term “organism” refers to a living thing which, in at least some form, is capable of responding to stimuli, reproduction, growth or development, or maintenance of homeostasis as a stable whole (e.g., an animal). The organism may be composed of many cells which may be grouped into specialized tissues or organs.

“Sample” or “biological sample” refers to any solid or liquid sample isolated from a live being. In a particular embodiment, it refers to any solid (e.g., tissue sample) or liquid sample isolated from an animal (e.g., human), such as a biopsy material (e.g., solid tissue sample), blood (e.g., plasma, serum or whole blood), saliva, synovial fluid, urine, amniotic fluid and cerebrospinal fluid. Such sample may be, for example, fresh, fixed (e.g., formalin-, alcohol- or acetone-fixed), paraffin-embedded or frozen prior to analysis of AID expression level. In an embodiment, the above-mentioned sample is obtained from a tumor.

As used herein, the term “tissue” or “tissue sample” refers to a group of cells, not necessarily identical, but from the same origin, that together carry out a specific function. A tissue is a cellular organizational level intermediate between cells and a complete organism. Organs are formed by the functional grouping together of multiple tissues. Examples of tissues include dermal, adipose, connective tissue, epithelial, muscle, nervous tissues. Other examples of biological tissues include blood cells populations (e.g., B or T lymphocytes populations), breast or ovarian tissues.

Similarly, the expression “reference gene expression and/or activity of a gene” refers to the expression and/or activity of that gene used as a control for the measure performed in a sample from a subject. “Reference gene sample” as used herein refers to a sample comprising a reference expression and/or activity of a gene.

More particularly, the expression “reference UNG expression and/or activity” and “reference AID expression and/or activity” and “reference MMR expression and/or activity” refers to the AID expression and/or activity, or UNG expression and/or activity, or MMR expression and/or activity, respectively, used as a control for the measure performed in a sample from a subject. “Reference UNG sample” or “reference AID sample” or “reference MMR sample” as used herein refers to a sample comprising a reference UNG expression and/or activity or reference AID expression and/or activity or reference MMR expression and/or activity, respectively.

Depending on the type of assay performed, the reference UNG expression and/or activity or reference AID expression and/or activity or reference MMR expression and/or activity can be selected from an established standard, a corresponding UNG or AID or MMR expression and/or activity, respectively, determined in the subject (in a sample from the subject) at an earlier time; a corresponding UNG or AID or MMR expression and/or activity, respectively, determined in one or more control subject(s) known to not being predisposed to an AID-associated neoplastic disease, known to not having an AID-associated neoplastic disease, or known to have a good prognosis; known to have a predisposition to an AID-associated neoplastic disease or known to have an AID-associated neoplastic disease (e.g., a specific tumor subtype) or known to have a poor prognosis. In another embodiment, the reference UNG expression and/or activity or reference AID expression and/or activity or reference MMR expression and/or activity is the average or median value obtained following determination of UNG or AID or MMR expression or activity, respectively, in a plurality of samples (e.g., samples obtained from several healthy subjects or samples obtained from several subjects having an AID-associated neoplastic disease (e.g., cancer)).

Similarly, the reference expression and/or activity of a gene known to regulate AID mutator activity by controlling or repairing DNA damage can be selected from an established standard, a corresponding expression and/or activity determined in one or more control subject(s) known to not being predisposed to an AID-associated neoplastic disease, known to not having an AID-associated neoplastic disease, or known to have a good prognosis; known to have a predisposition to an AID-associated neoplastic disease or known to have an AID-associated neoplastic disease or known to have a poor prognosis. In another embodiment, the reference expression and/or activity of a gene known to regulate AID mutator activity by controlling or repairing DNA damage is the average or median value obtained following determination of expression or activity of the gene known to regulate AID mutator activity by controlling or repairing DNA damage in a plurality of samples (e.g., samples obtained from several healthy subjects or samples obtained from several subjects having an AID-associated neoplastic disease (e.g., cancer)).

“Corresponding normal tissue” or “corresponding tissue” as used herein refers to a reference sample obtained from the same tissue as that obtained from a subject. Corresponding tissues between organisms (e.g., human subjects) are thus tissues derived from the same origin (e.g., two B lymphocyte populations).

Measurement of UNG or AID in a sample

The present invention encompasses methods comprising detecting the presence of UNG and/or AID and/or MMR activity and/or expression in a subject sample. In a specific embodiment, the present invention encompasses detecting the presence of UNG and AID activity and/or expression in a subject sample. In another specific embodiment, the present invention encompasses detecting the presence of UNG and MMR activity and/or expression in a subject sample. In another specific embodiment, the present invention encompasses detecting the presence of UNG and AID and MMR activity and/or expression in a subject sample.

In another embodiment, the present invention encompasses methods comprising determining whether UNG and/or AID and/or MMR activity and/or expression in a subject sample is substantially similar to that in a reference expression and/or activity. In a specific embodiment, the present invention encompasses determining whether UNG and AID activity and/or expression in a subject sample is substantially similar to that in a reference expression and/or activity. In a specific embodiment, the present invention encompasses determining whether UNG and MMR activity and/or expression in a subject sample is substantially similar to that in a reference expression and/or activity. In a specific embodiment, the present invention encompasses determining whether UNG and AID and MMR activity and/or expression in a subject sample is substantially similar to that in a reference expression and/or activity

In another embodiment, the present invention encompasses methods comprising determining whether UNG and/or AID and/or MMR activity and/or expression in a subject sample is higher than a reference expression and/or activity. In a specific embodiment, the present invention encompasses determining whether UNG and AID activity and/or expression in a subject sample is higher than that in a reference expression and/or activity. In a specific embodiment, the present invention encompasses determining whether UNG and MMR activity and/or expression in a subject sample is higher than that in a reference expression and/or activity. In a specific embodiment, the present invention encompasses determining whether UNG and AID and MMR activity and/or expression in a subject sample is higher than that in a reference expression and/or activity

The present invention also encompasses method comprising determining whether UNG or AID or MMR expression in B cells of a subject sample is substantially similar to a reference expression but in the context of an independent predisposing condition (e.g., (a) a reduced capacity for controlling/preventing/repairing DNA damage; and/or (b) a deficiency in specific DNA repair enzymes known to repair uracil in DNA) which results from a genetic mutation leading to an increase of the mutator activity of AID in the B cells (e.g., a loss-of-function mutation in TP53, ATM, or UNG2).

In cases where the reference UNG or AID or MMR sample is from the subject at an earlier time; from subject(s) known not to being predisposed to an AID-associated neoplastic disease, known not to have an AID-associated neoplastic disease, or known to have a good prognosis, an increased/higher UNG and/or AID and/or MMR expression and/or activity, respectively in the sample from the subject relative to the reference UNG and/or AID and/or MMR expression and/or activity, respectively, is indicative that the subject has an AID-associated neoplastic disease, has a predisposition to an AID-associated neoplastic disease (e.g., has a higher risk of developing an AID-associated neoplastic disease and/or of experiencing an AID-associated neoplastic disease progression) or has a poor prognosis (e.g., lower survival probability, higher probability of AID-associated disease recurrence), while a comparable or lower expression or activity in a sample from the subject relative to the reference expression and/or activity is indicative that the subject does not have an AID-associated neoplastic disease, is not predisposed to an AID-associated neoplastic disease or has a good prognosis (e.g., higher survival probability, lower probability of cancer recurrence).

In cases where the reference UNG or AID or MMR sample is from subject(s) known to have a predisposition to an AID-associated neoplastic disease, known to have an AID-associated neoplastic disease or known to have a poor prognosis, a comparable or increased/higher UNG and/or AID expression and/or activity, respectively, in a sample from the subject relative to the reference UNG and/or AID expression and/or activity, respectively, is indicative that the subject has an AID-associated neoplastic disease, has a predisposition to an AID-associated neoplastic disease or has a poor prognosis (e.g., lower survival probability, higher probability of AID-associated disease recurrence), while a lower expression or activity in a sample from the subject relative to the reference expression and/or activity is indicative that the subject does not have an AID-associated neoplastic disease, is not predisposed to an AID-associated neoplastic disease or has a good prognosis (e.g., higher survival probability, lower probability of AID-associated neoplastic disease recurrence).

As used herein, a “higher” or “increased” level refers to levels of expression or activity in a sample (i.e. sample from the subject) which exceeds with statistical significance that in the reference sample (e.g., an average corresponding level of expression or activity a healthy subject or of a population of healthy subjects, or when available, the normal counterpart of the affected or pathological tissue) measured through direct (e.g., Anti-AID antibody, Anti-UNG antibody quantitative PCR) or indirect methods. The increased level of expression and/or activity refers to level of expression and/or activity in a sample (i.e. sample from the subject) which is at least 10% higher, in an other embodiment at least 15% higher, in an other embodiment at least 20% higher, in an other embodiment at least 25%, in an other embodiment at least 30% higher, in a further embodiment at least 40% higher; in a further embodiment at least 50% higher, in a further embodiment at least 60% higher, in a further embodiment at least 100% higher (i.e. 2-fold), in a further embodiment at least 200% higher (i.e. 3-fold), in a further embodiment at least 300% higher (i.e. 4-fold), relative to the reference expression and/or activity (e.g., in corresponding normal adjacent tissue or alternatively, in a define group of subject).

As used herein, a “substantially similar level” refers to a difference in the level of expression or activity between the level determined in a first sample (e.g., sample from the subject) and the reference expression and/or activity which is less than about 10%; in a further embodiment, 5% or less, in a further embodiment, 2% or less.

As used herein, “aberrant AID expression and/or activity” refers to an increased expression of AID compared to equivalent normal tissue or to the presence of AID expression and/or activity in a tissue not normally expressing AID.

As used herein the term “AID-positive tissue” refers to tissue containing cells in which expression and/or activity AID is detectable.

As used herein the term “AID expressing neoplastic cells” refers to neoplastic cells (e.g., tumor) in which expression and/or activity AID is detectable.

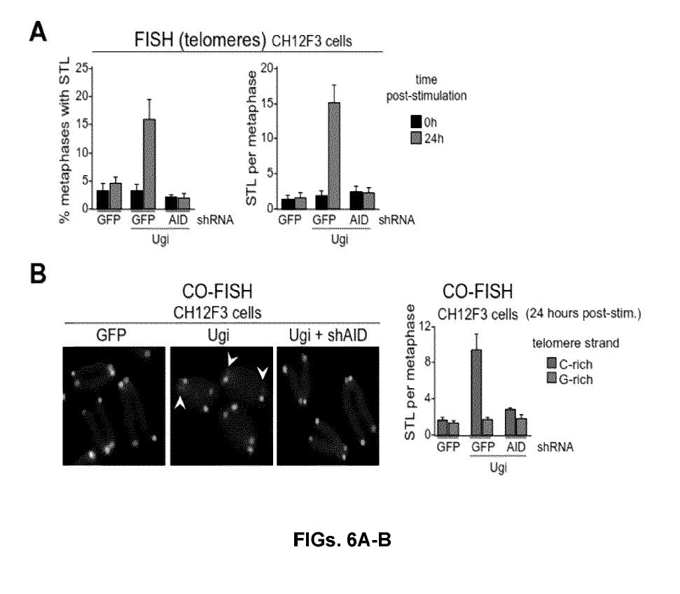

Methods for measuring AID and/or UNG and/or MMR expression and/or activity are well known. See in particular under title “UNG expression or AID expression or MMR expression” above. Without being so limited, such methods are described in FIG. 6. Additional methods for measuring UNG are described by Di Noia ⁶⁹.

Subjects stratification methods

The methods of the present invention may also be used for classifying or stratifying a subject into subgroups based on UNG and/or AID and/or MMR (in a specific embodiment, UNG and AID; or UNG and MMR; or most specifically UNG and AID and MMR) expression and/or activity enabling a better characterization of the subject disease and eventually a better selection of treatment depending on the subgroup to which the subject belongs.

In one aspect, the present invention provides a method for stratifying a subject, said method comprising: (a) detecting/determining the expression and/or activity of AID and/or UNG and/or MMR (e.g., MSH2) in a sample from the subject, and optionally (b) comparing said expression and/or activity to a reference expression and/or activity; and (c) stratifying said subject based on said detection and/or said comparison.

The invention provides a method for stratifying a subject based on the expression and/or activity of AID and/or UNG and/or MMR (e.g., MSH2) as determined in a tissue sample (e.g., a biopsy) from the subject using the assays/methods described herein.

In another aspect, the present invention provides a method for stratification of a subject having cancer, said method comprising: (a) detecting an expression and/or activity of AID and/or UNG and/or MMR (in a specific embodiment, UNG and AID; or UNG and MMR; or most specifically UNG and AID and MMR) in a sample (e.g., a tumor sample) from the subject, and (b) stratifying said subject based on said detection or absence of detection; wherein the detection (i.e. presence) in said sample is indicative that said subject is suitable for a treatment with an UNG inhibitor of the present invention.

Combination of therapies

In an embodiment, the above-mentioned prevention/treatment comprises the use/administration of more than one (i.e. a combination of) therapies (e.g., active/therapeutic agent (e.g., an agent capable of inhibiting AID expression and/or activity)). The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one prophylactic or therapeutic agent in the course of a coordinated treatment to achieve an improved clinical outcome. Such co-administration may also be coextensive, that is, occurring during overlapping periods of time. For example, a first agent may be administered to a subject before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat the disorder in question (e.g., an antineoplastic agent).

Currently used combined therapies for treating cancer include the administration of radiation therapy with therapeutic antineoplastic agents.

UNG inhibitors combined treatment in AID expressing cells

In one embodiment, the treatment of AID-positive neoplastic cells with a compound reducing the expression and/or activity of UNG is combined with at least one other active agent (e.g., antineoplastic agent in order to reduce tumor progression and/or development of drug resistance).

In an embodiment, the UNG inhibitor is used in combined therapy with an agent favoring AID nuclear localization such as an inhibitor of elongation factor eukaryotic elongation factor 1 α (eEF1A), an enzyme that retains AID in the cytoplasm. Without being so limited such eEF1A include didemnin B (DidB) and cytotrienin A (CytA)).

In one embodiment for the treatment of an AID-expressing neoplastic disease, at least one UNG inhibitor is used in combined chemotherapy for the treatment of AID-positive cancer. In specific aspects of the present invention, an UNG inhibitor is combined to at least one of Bay 43-9006, paclitaxel, gemcitabine, cisplatin, docetaxel (Taxol™) (Taxotere™), and AraC for the treatment of AID-positive solid tumors or to imatinib mesylate (Gleevec) for subjects with AID-positive chronic myeloid leukemia (CML) or AID-positive ALL. Additional antineoplastic agents may also include one or more of cyclopamine, CUR0199691, Etoposide, Camptothesin, Cisplatin™, Oxaliplatin™ and their derivatives, cyclophosphamide compound (Cy), 13-cis retinoic acid, histone deacetylase inhibitor (SAHA), nucleotide analogues (e.g., 5-fluoro uracyl, azacitidine (Vidaza), Gemcitabine (Gemzar), cytarabine (Ara-C)), kinase inhibitors (e.g., imatinib), etc

### Dosage

The amount of the agent or pharmaceutical composition which is effective in the prevention and/or treatment of a particular disease, disorder or condition (e.g., neoplastic disease) will depend on the nature and severity of the disease, the chosen prophylactic/therapeutic regimen (i.e., compound, DNA construct, protein, cells), systemic administration versus localized delivery, the target site of action, the patient's body weight, patient's general health, patient's sex, special diets being followed by the patient, concurrent medications being used (drug interaction), the administration route, time of administration, and other factors that will be recognized and will be ascertainable with routine experimentation by those skilled in the art. The dosage will be adapted by the clinician in accordance with conventional factors such as the extent of the disease and different parameters from the patient. Typically, 0.001 to 1000 mg/kg of body weight/ of subject per day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a subject, in the context of the present invention should be sufficient to effect a beneficial prophylactic and/or therapeutic response in the patient over time. The size of the dose will also be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

Adjustment of dose of UNG inhibitors

In one embodiment of the present invention, the dose of the at least one UNG inhibitor administered to inhibit UNG expression and/or activity, is adjusted to the level of UNG in the sample (e.g., tumor tissue).

In another aspect, the present invention provides a method for adjusting a treatment, for example the dose of an UNG inhibitor to administer to a subject. Such method comprising: (a) determining the expression and/or activity of UNG in a sample from said patient; (b) comparing said expression and/or activity to a reference expression and/or activity of UNG determined in a biological sample obtained from said patient at an earlier time (e.g., at the start of treatment); wherein a decrease in said expression and/or activity relative to a corresponding expression and/or activity of UNG determined in a biological sample obtained from said patient at an earlier time (at the start of treatment) is indicative that the dose of the at least one UNG inhibitor administered is appropriate whereas a similar level or an increase of UNG expression over time is indicative that the dose of the at least one UNG inhibitor administered to the subject should be increased.

Pharmaceutical composition

The invention also provides a pharmaceutical composition (medicament) comprising at least one agent of the invention (e.g., an UNG inhibitor) (alone or in combination with another agent- sea combined treatment above), and a pharmaceutically acceptable diluent, carrier, salt or adjuvant. Such carriers include, for example, saline, buffered saline, dextrose, water, glycerol, ethanol, and combinations thereof. The pharmaceutical composition may be adapted for the desired route of administration (e.g., oral, sublingual, nasal, parenteral, intravenous, intramuscular, intraperitoneal, aerosol).

Kit or package

The present invention also provides a kit or package comprising the above-mentioned inhibitor or pharmaceutical compositions. Such kit may further comprise, for example, instructions for the prevention and/or treatment of an AID-associated disease (e.g., neoplastic disease (e.g., B-cell lymphomas)), containers, devices for administering the agent/composition, etc.

The present invention also provides a kit or package comprising a reagent useful for determining UNG expression and/or activity and/or AID expression and/or activity and/or MMR expression and/or activity (e.g., a ligand that specifically binds AID or UNG or MMR polypeptide such as an anti-AID or anti-UNG or anti-MMR antibody, or a ligand that specifically binds a AID or UNG or MMR nucleic acid such as an oligonucleotide). Such kit may further comprise, for example, instructions for the prognosis and/or diagnosis of cancer, control samples, containers, reagents useful for performing the methods (e.g., buffers, enzymes), etc.

As used herein the term “subject” is meant to refer to any animal, such as a mammal including human, mice, rat, dog, cat, pig, cow, monkey, horse, etc. In a particular embodiment, it refers to a human.

A “subject in need thereof” or a “patient” in the context of the present invention is intended to include any subject that will benefit or that is likely to benefit from the decrease in the expression or activity of AID. In an embodiment, a subject in need thereof is a subject diagnosed as expressing (e.g., overexpressing) AID in normal or tumor cells.

As used herein, the term “a” or “the” means “at least one”.

Although the present invention has been described hereinabove by way of specific embodiments thereof, it can be modified, without departing from the spirit and nature of the subject invention as defined in the appended claims.

The present invention is illustrated in further details by the following non-limiting examples.

### EXAMPLE 1

### Materials and Methods

Mice, mouse cohorts and immunization.

C57BL6/J mice were from Jackson labs (Bar Harbor, Me.). and NRG mice were from Jackson labs (Bar Harbor, Me.). AID-GFPtg mice- ⁶, a gift of Dr R Casellas (NCI, Bethesda, Md.), Aicda−/− mice ⁵⁹, a gift of Dr T Honjo (Kyoto University, Japan) and Ung−/−⁶⁰, a gift from Dr H Krokan (NUST, Trondheim, Norway) were all in C57BL6/J background. Aicda−/− Ung−/− mice were bred at the IRCM animal facility. Experimental cohorts for lymphoma follow up were observed daily for spontaneous signs of malaise or visible tumors and sacrificed when reaching one of the predefined endpoints or at 30 months old. Spleens, enlarged lymph nodes and/or any other tumors were harvested at necropsy and prepared for histology or analyzed by flow cytometry. Where indicated, mice were immunized with 50 ug of NP18-CGG (Biosearch Technologies) in Imject™ Alum adjuvant (Thermo Scientific) intra-peritoneally and analyzed by flow cytometry 8 days later. All experiments were approved by the Animal protection committee at the IRCM, according to the guidelines of the Canadian Council on Animal Care.

Histological analysis

Tissues were fixed with 4% formaldehyde overnight at room temperature and embedded in paraffin. Sections were stained with H&E. IHC was performed on 5-μm sections of paraffin-embedded tissues, de-paraffinized, rehydrated, and subjected to antigen retrieval. After blocking endogenous peroxidase activity with 3% hydrogen peroxide, sections were incubated overnight with rat anti-mouse CD45R (1:50; BD Pharmingen) at 4° C. and/or AID (1:100 ; eBioscience) at 37° C. followed by goat anti-rat IgG-HRP (1:400; Vector Laboratories) or goat bio-anti-rabbit IgG (1:200; Vector Laboratories), or directly with Biotinylated peanut agglutinin (PNA, 20 [symbol] g/ml; Vector Laboratories) for 60 min at RT and developed using avidin, bio-HRP and HRP substrate included in the ImmPACT™ NovaRED™ HRP substrate (Vector Laboratories). Formalin fixed and paraffin embedded tissues were analyzed by a pathologist after H&E and IHC staining. For visualization of AID-GFP in the spleen of AID-GFPtg mice, organs were embedded in CryoMatrix (Thermo Scientific) and snap frozen before sectioning. Sections were fixed in 3.7% (WN) paraformaldehyde for 10 min, permeabilized with 0.5% Triton-X for 10 min, washed, and incubated for 15 min with DAPI before mounting in Aqua Mount (Thermo Scientific). Images of the whole tissue section was composed multiple fields imaged using an LSM700 confocal microscope (Zeiss), and the GFP+area was scored in ImageJ software (NIH).

Flow cytometry

Mononuclear cells from mouse spleens or other organs were extracted using a cell strainer and stained with anti-B220-APC and anti-CD3-PE, anti-CD21-FITC, anti-CD23- PE, IgD-FITC (all BD Pharmingen) anti-IgM-PE (Invitrogen), and biotin-Ig kappa/lambda light chain followed by anti-biotin-PE-Vio770 (Miltenyi Biotech). Leukemic cells from NRG mice transplanted with infected BM were stained with rat anti-mouse B220-APC (BD Biosciences). Propidium iodide was used to gate out dead cells. Results were acquired using a BD LSR I (BD biosciences) and analyzed using FlowJo™.

For the BrdU incorporation analysis CH12F3 cells (2×10⁵/ml) were incubated for 30 min in culture medium containing 10 μM BrdU. Then, cells were harvested, washed twice with PBS, and fixed in cold 70% ethanol ON at 4 C. After removal of ethanol, DNA was denatured with 2 N HCl/0.5% Triton X-100 for 30 min at RT, then neutralized with two washes of 0.1 M sodium tetraborate (pH 9), and resuspended in 70% ethanol. Cells were recovered by centrifugation, washed once with PBS and resuspended in 100 [symbol] l of blocking buffer (0.5% Tween-20, 1% BSA in PBS) containing 10 μl mouse anti-BrdU antibody (Becton Dickinson), and incubated at RT for 30 min. After a wash with PBS, cells were incubated 15 min at RT with goat anti-mouse Alexa 647 antibody diluted in blocking buffer. Finally cells were washed with PBS once and resuspended in PBS containing 5 μg/ml propidium iodide and analyzed using an Accuri flow™cytometer.

### RT-PCR

RNA was isolated from cultured BM and leukemic cells using TRIzol™(Life Technologies) and cDNA synthesized using the ProtoScript™ M-MuLV Taq RT-PCR kit (New england Biolabs). End point PCR was performed on the cDNA for 35 cycles [940C 30 sec, 550C 30 sec, 680C 1 min] and 30 cycles [940C 30 sec, 510C 20 sec, 680C 1 min] using Taq DNA polymerase with primers fwd GTGCCACCTCCTGCTCACTGG (SEQ ID NO: 30) and rev TTCATGTAGCCCTTCCCAGGC (SEQ ID NO: 31) for mouse AID (490 bp PCR product) and fwd ACTCCACTCACGGCAAATTCA (SEQ ID NO: 32) and rev GCCTCACCCCATTTGATGTT (SEQ ID NO: 33) for mouse GAPDH (121 bp PCR product).

Cell lines and CSR Analysis

B cell lymphoma cells, CH12F3-2 cells, were cultured in RPMI medium supplemented with 10% FBS, 0.05% 2- mercaptoethanol and 5% NCTC 109 (Sigma). CH12F3-2 cells stably expressing Ugi were obtained by retroviral delivery using pMIG vector as described before ³⁹. Class switching induction to IgA in CH12F3-2 cells was performed as previously published ³⁹. CSR to IgA in CH12F3-2 cells was determined with an Accuri C6™ Flow Cytometer (BD Biosciences). Mouse B cells were purified from freshly isolated splenocytes using anti-CD43 magnetic beads as described ⁷⁰. CSR was induced using 5 [symbol] g/ml LPS (Sigma) and 20 ng/ml IL-4 (Preprotech). Cells were harvested for ChIP or metaphase spreads and FISH at 24 h post stimulation.

Chromatin immunoprecipitation assays

ChIPs to evaluate interaction with the telomeric chromatin or the Ig locus were performed as described previously ^{39,61}. Briefly, cells were cross-linked with 1% formaldehyde for 20 min at room temperature and reaction was stopped by addition of glycine to 125 mM final concentration. Cells were washed twice with cold PBS, harvested and kept at −80° C. O.N. Samples were resuspended in RIPA buffer (150 mM NaCl, 1% [v/v] Igepal CA-630, 0.5% [w/v] Sodium Deoxycholate, 0.1% [w/v] SDS, 50 mM Tris-HCl at pH 8, 5 mM EDTA, protease and phosphatase inhibitors) and sonicated to generate DNA fragments <500 bp using a Bioruptor™ Next Gen (Diagenode).

In order to increase the sensitivity of the ChIP assays and evaluate the presence of AID at the telomeres we performed the immunoprecipitations with fractions enriched in crosslinked chromatin. For this goal B cells fixed with 1% formaldehyde as described above were resuspended in RIPA buffer, sonicated during 90 seconds using a Bioruptor™ Next Gen (Diagenode), and the lysates were then spun down briefly (2000g, for 5 min at 4 C) to remove debris. Then sucrose was added to the extract (5% final concentration) and layered onto 20% sucrose in RIPA buffer for ultracentrifugation at 40k rpm for 1.5 h using a Beckman™ rotor TLA-100. The chromatin-enriched pellets were then sonicated to generate DNA fragments <500 bp as described above and used for the immunoprecipitation step. Samples were then clarified by centrifugation at 20,000 RPM (4° C.) and their protein content was measured using the BCA method (Biorad).

For immunoprecipitation, 0.5 mg (2mg/ml) of protein extract was pre-cleared for 2 h with 30 μL of 50% G protein-Sepharose™ slurry before addition of indicated antibodies. Between two and five micrograms of each antibody was added to the samples and incubated overnight at 4° C. Immunocomplexes were eluted from Agarose NG plus (Santa Cruz Biotech) for 10 min at 65° C. with 100 μl of Elution buffer (1% [w/v] SDS), and cross-linking was reversed by adjusting to 200 Mm NaCl, 1 mM EDTA, 1mM DTT) and incubating ON at 65° C. in the presence of 5 μg proteinase K. DNA was purified using QlAquick™ PCR purification kit (Qiagen), and DNA resuspended in 60 μl of Tris-HCl pH 8 was used as template in real-time PCR reactions to evaluate co-immunoprecipitation of Ig DNA 39, or Southern blot with a telomeric probe or ALU repeat probe as described previously ⁶¹. IgG and input DNA values were used to subtract/normalize the values from ChIP samples. All primer sequences used for the ChIP analyses are available upon request.

### FISH

For metaphase analysis cells were incubated with 50 ng/ml colcemide in cell culture media for 3 hours, harvested by trypsinization, incubated for 10 min at RT in 75 mM KCl, and fixed in freshly prepared methanol:glacial acetic acid (3:1 v/v). Cells were stored at 4° C. and when needed dropped onto wet slides and air-dried. For FISH analysis of the metaphases the cells were pre-treated with 0.05% w/v pepsin in 10 mM HCl during 10 minutes at 37° C. After washes with 1X PBS, cells were fixed with 1% formaldehyde in 1X PBS during 10 minutes at RT, washed again with 1X PBS and dehydrated with ethanol series (70-90-100%, 2 min each at RT) and air-dried ON. Then, cells were denatured with hybridization solution (70% deionized formamide; 2.5% 50× Denhardt solution; 10 mM Tris pH 7.5; 1.5 mM MgCl₂) containing Alexa488-conjugated PNA probe (Alexa488-OO-(CCCTAA)₃) 2 min at 80° C. on a heat block. After 4 hours incubation at RT in the dark, the samples were washed twice with wash solution (70% deionized formamide and 10 mM Tris-HCl [pH 7.2]) at RT and then twice with PBS. For DAPI staining of DNA, slides with metaphase spreads were incubated 10 min in 0.5 [symbol] g/ml 4′, 6-diamino-2-phenylindole (DAPI) (Sigma) in PBS, washed with PBS for 2 min, and mounted in SlowFade™ Gold antifade mounting reagent (Life Technologies). Finally, the samples were analyzed as described above. Where indicated, metaphases were spread as described ⁷¹.

Anaphase cells were visualized by DAPI staining of cells fixed with 2% paraformaldehyde in 1X PBS and attached to slides pre-treated with poly-lysine and analyzed as described previously ³³.

### CO-FISH

CO-FISH was performed as described ³³, with the variation that cells were incubated with BrdU and BrdC simultaneously for 16 hours, and hybridization was performed with CY5-OO-(TTAGGG)₃ and Alexa488-OO-(CCCTAA)₃ probes (Panagene, KOREA). In brief, BrdU and BrdC are incorporated into chromosomes throughout one S-phase, metaphases are spread on slides, the BrdU substituted DNA strands are degraded with Exonuclease III and the remaining strands hybridized with fluorescence-labeled DNA probes of different colors, specific either for the G-rich telomere strand ([TTAGGG]n, polymerized by lagging strand synthesis), or the C-rich telomere strand ([CCCTAA]n, polymerized by leading strand synthesis). Hybridization with PNA probes and DAPI staining were performed as described above for FISH analysis. The resulting chromosomes show dual staining and allow distinction between leading and lagging strands. Chromosomes were visualized using a Leica™ DMI6000B microscope.

Antibodies sources and Western blot

Western blot for the different proteins was performed as previously described ⁶¹. The following antibodies were used in this study: Actin (Sigma Aldrich, A2066); AID (Active Motif, 39885); EXO1 (Novus Biologicals, NBP1-19709); yH2AX (EMD Millipore, 05-636); MSH2 (Santa Cruz Biotech sc-22771); SPT5 (Santa Cruz Biotech sc-28678).

shRNA knockdowns

Stable shRNA-mediated knockdowns were generated in CH12F3-2 cells following protocols from the RNAi Consortium (TRC). Transfected cells were selected with puromycin at 1 μg/ml final concentration. For double knockdowns, CH12F3-2 cells were first transfected with a pLKO.1 neomycin version of the shRNA (shAID or shGFP) and selected, then transduced with the second shRNA and selected again with puromycin before analysis. The shRNAs used herein are listed in Table IV. CH12F3 Ugi cells transduced with pMIG-Ugi-IRES-GFP were generated as described ³⁹, and human lymphoma Ugi cells were generated by retroviral delivery using a pLPC-PURO system (pLPC-Ugi-PURO) and transduced cells were selected with 1 ug/m1 of puromycin. The Ugi protein from bacteriophage PBS2 and its ability to inhibit eukaryotic UNG have been described ^{69,31}.

TABLE IV
shRNAs used herein.

| shRNA | Species | Sequence(5′ to 3′) |
|---|---|---|
| AID-1 | Mouse | GCGAGATGCATTTCGTATGTT (SEQ ID NO: 34) |
| AID-2 | Mouse | GAAGTCGATGACTTGCGAGAT (SEQ ID NO: 35) |
| hAID-1 NM_020661.1-211s1c1 | Human | CTTTGGTTATCTTCGCAATAA (SEQ ID NO: 36) |
| hAID-2 M_020661.1-485s1c1 | Human | GCCATCATGACCTTCAAAGAT (SEQ ID NO: 37) |
| TERT NM_009354.1-1011s1c1 | Mouse | CCATTTATTGAGACCAGACAT (SEQ ID NO: 38) |
| hMSH2-1 | Human | GCCTTGCTGAATAAGTGTAAA (SEQ ID NO: 39) |
| NM_000251.1-1050s1c1 | | |
| hMSH2-2 | Human | CCTGGCAATCTCTCTCAGTTT (SEQ ID NO: 40) |
| NM_000251.1-441s1c1 | | |
| MSH2-1 | Mouse | CGAGATCATTTCACGGATAAA (SEQ ID NO: 41) |
| NM_008628.1-2811s1c1 | | |
| MSH2-2 | Mouse | GCTCACTTAGACGCCATTGTT (SEQ ID NO: 42) |
| NM_008628.1-1837s1c1 | | |
| GFP | — | GCAAGCTGACCCTGAAGTTCAT (SEQ ID NO: 43) |

UNG activity assay

UNG activity assay was performed as previously described ⁶⁹ with minor modifications. Briefly, exponentially growing cells were washed in 1X PBS buffer, resuspended in HED buffer (25 mM Hepes, 5 mM EDTA, 1 mM dithiothreitol and 10% glycerol at pH 7.8) with complete protease inhibitors (Roche) and lysed by sonication (five pulses of 30 seconds) in a Bioruptor™ (Diagenode). After centrifugation at 14,000 rcf for 20 min at 4° C., the supernatant was frozen in aliquots into liquid nitrogen and stored at 80° C. UNG assays were carried out in HED buffer by mixing 10 μg of cell extract with 1 pmol of fluorescein-labeled oligonucleotide substrate in a final volume of 10 μl for 3 hours at 37° C. The double-stranded oligonucleotide with a single dU/dG mismatch was made by annealing 5′-ATTATTATTATTCCGUGGATTTATTTATTTATTTATTTATTT(SEQ ID NO: 45)-fluorescein (FITC) to the complementary oligonucleotide, 5′-AAATAAATAAATAAATAAATAAATCCGCGGAATAATAATAAT-3′(SEQ ID NO: 46). The reaction was terminated by the addition of 10 UI of formamide loading dye and the products were resolved on 15% TBE-urea polyacrylamide gels. The apyrimidinic endonuclease (APE) activity present in the extracts was sufficient to cleave all the abasic substrate generated by UNG during the reaction. The FITC signal of the reaction products was visualized using a Typhoon Phosphorlmager™ (GE Healthcare).

### EXAMPLE 2

### AID at the Telomeres in Activated B Cells

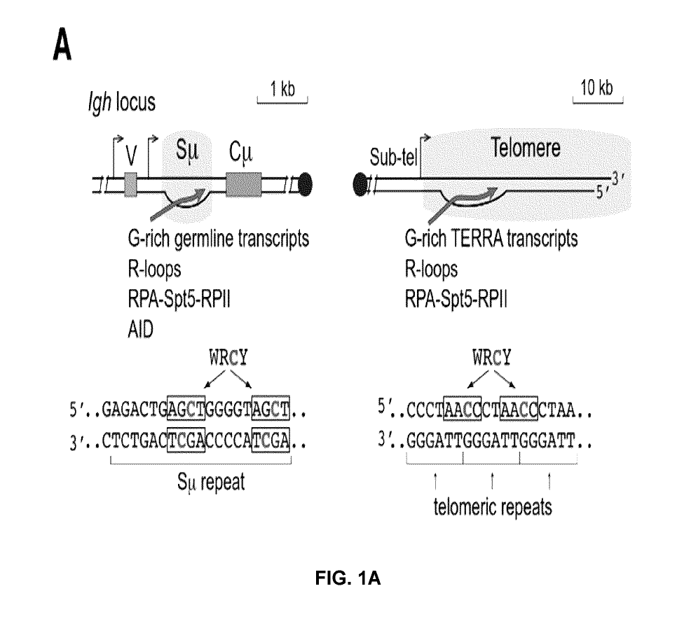

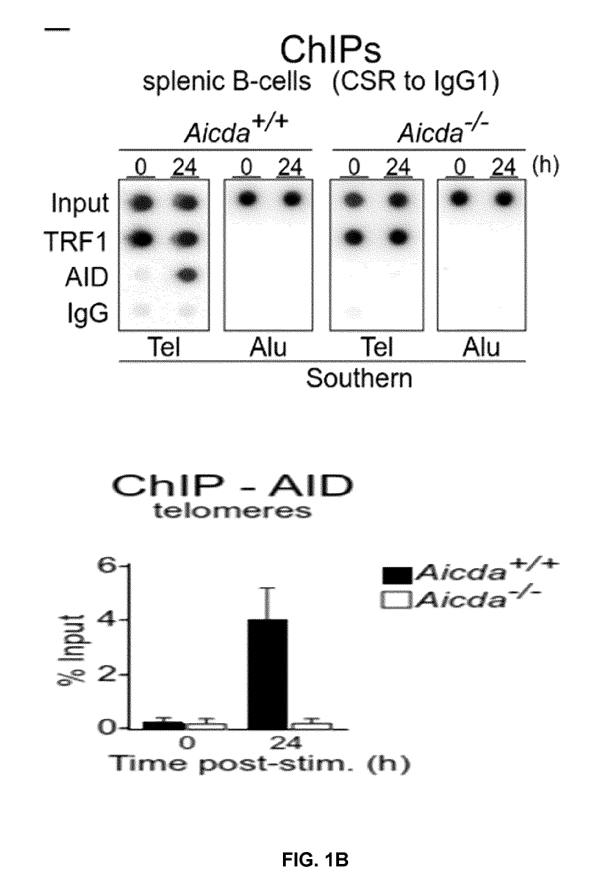

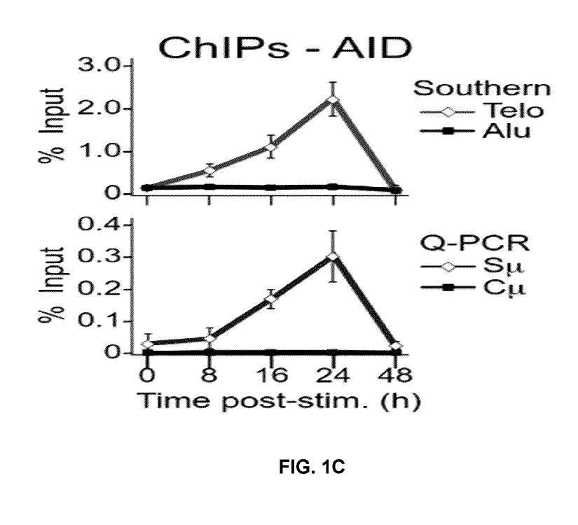

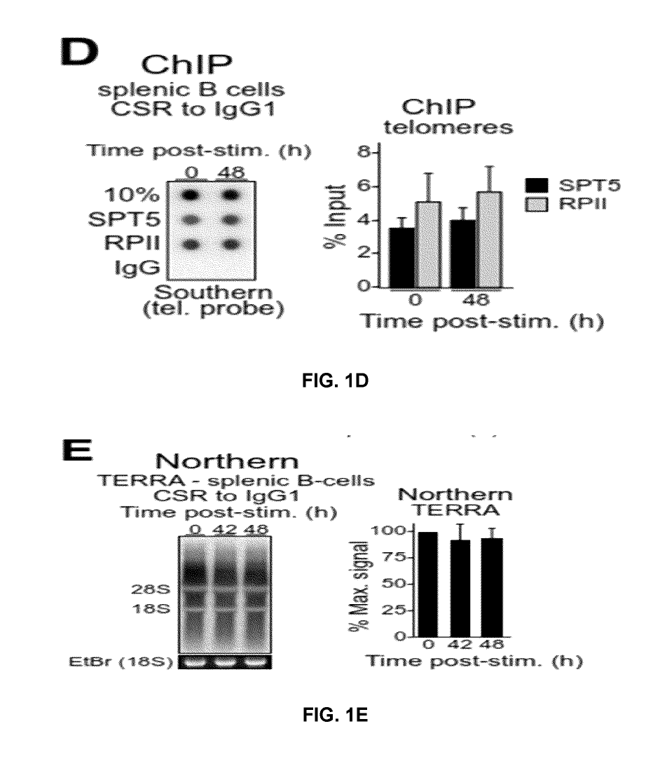

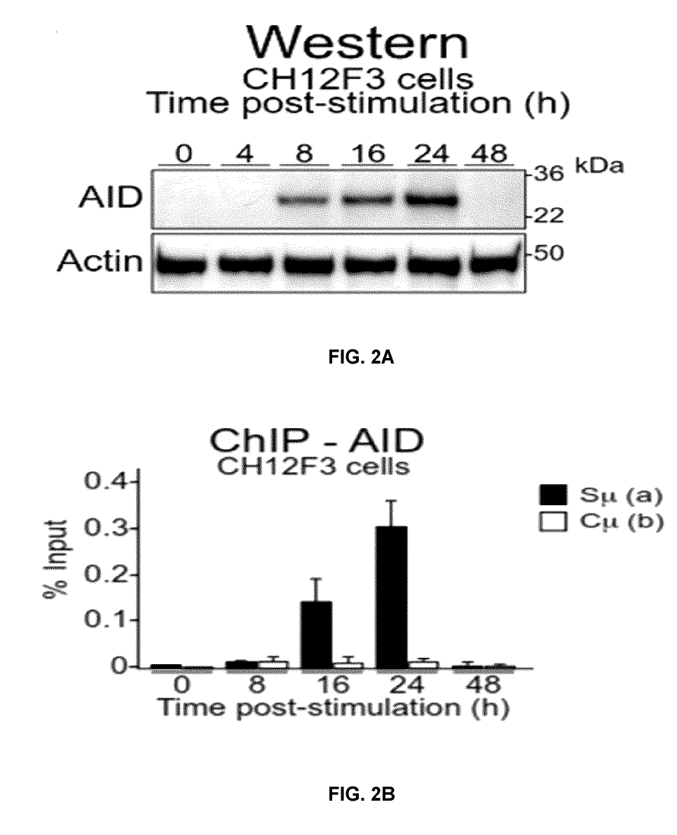

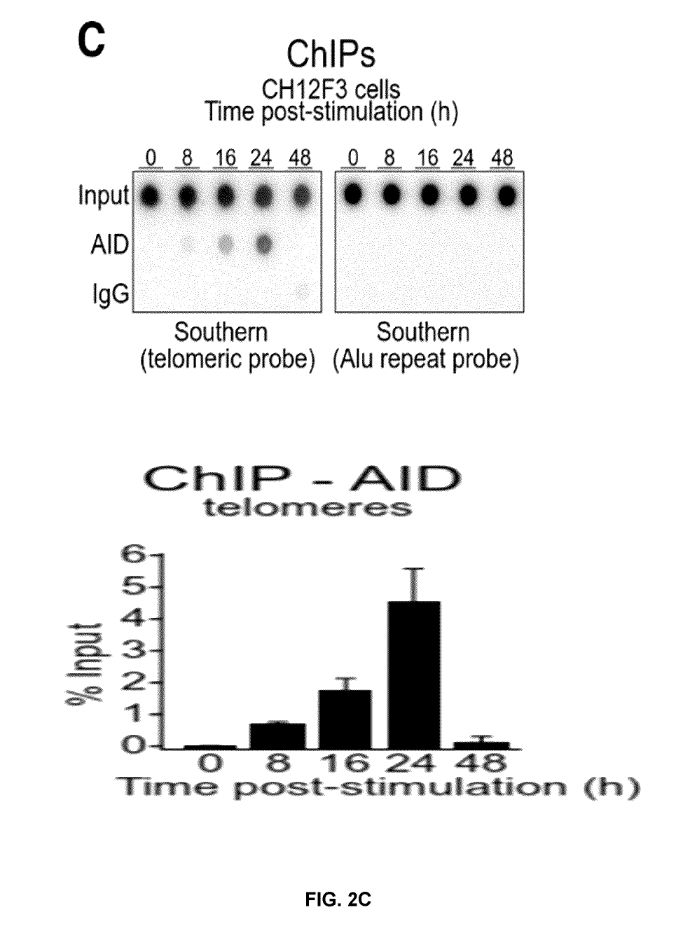

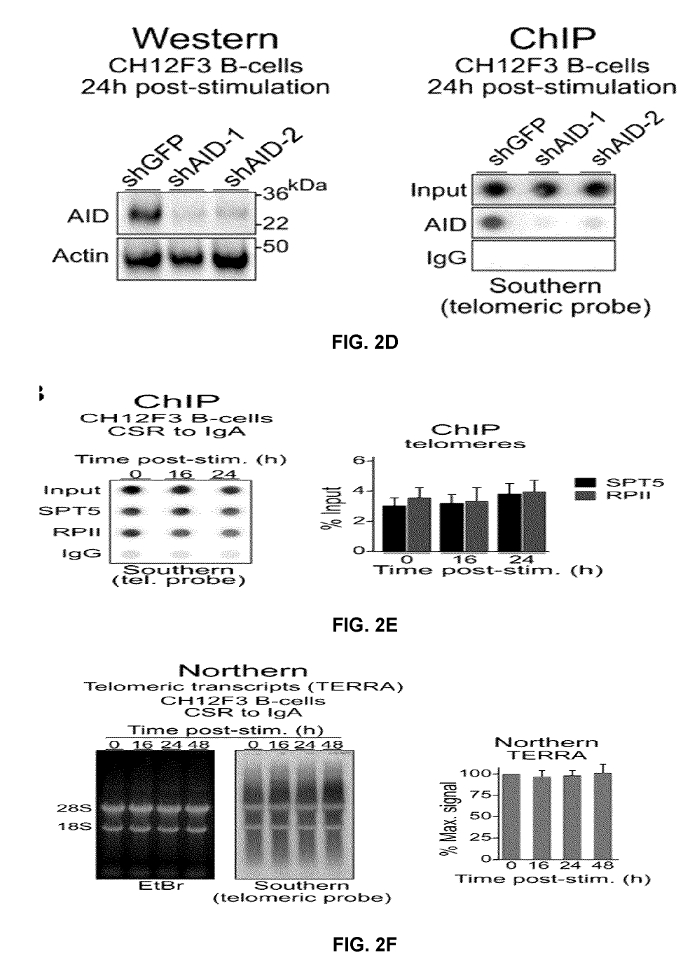

The results described above suggested that AID could target telomeric DNA during CSR. In support of this, an analysis of the telomeric DNA sequence revealed the presence of a putative AID hotspot target (5′-WRCY-3′) every two telomeric repeats (FIG. 1A). Telomeres and S-regions share many similarities: both are located downstream of an RNA polymerase II (RPII) promoter producing sterile transcripts, which plays a relevant role in AID recruitment to the chromatin ^{3-5,18,19,} and have C-rich template DNA strands enriched in AID hotspot sequences (FIG. 1A). However, unlike the DNA sequence in the S-regions of the immunoglobulin locus where hotspots for AID are present in both DNA strands, telomeres show those exclusively in the C-rich strand (FIG. 1A). Telomere transcription initiation occurs in the subtelomeric region, likely at all telomeres, and terminates within the telomeric tract, creating telomeric repeat-containing RNA or TERRA molecules that are very heterogeneous in length ^{47,48}. The inventors used chromatin immunoprecipitation (ChIP) assays with chromatin of the CH12F3 B cell lymphoma line and from mouse splenic B-cells stimulated for CSR. The inventors found that AID associated with similar kinetics to telomeres and SUI after CSR stimulation in both B cell types (FIGS. 1-2-). AID was also present at the S-region (Sμ) but not at the Eμ and Cμ regions of the immunoglobulin locus (consistent with previous data showing that AID-dependent DSBs are found in S-regions, but not in the Eμ and Cμ ³⁸⁻³⁸, or Alu repeats showing the specificity of the AID signal at the Sμ and the telomeres (FIGS. 1B-2). The lack of AID signal in ChIPs performed with AID-null B-cells stimulated for class switching also confirmed the specificity of the assay (FIGS. 1B, D). ChIPs with antibodies against the telomeric protein TRF1 were used as positive control (FIG. 1D). Similarly, ChIP assays with chromatin from CH12F3 B-cells showed that AID interacts with the Ig locus and the telomeres from ˜8h to ˜36h post-stimulation of CSR, which correlates with the higher concentration of AID in the cell as revealed by Western analysis (FIGS. 2A-C). The specificity of the AID signal in the ChIP assays was confirmed by knockdown of AID via shRNA (shAID) (FIG. 2D and ³⁹). In addition RPII and the transcription factor SPT5 are present at the telomeres before and during CSR in splenic B-cells and CH12F3 cells (FIG. 1D and not shown), suggesting that the transcription of the telomeric C-rich strand is not affected during CSR. Indeed, Northern blot analysis in splenic B-cells revealed that the levels of TERRA do not vary significantly after CSR stimulation (FIG. 1E). Depleting AID in CH12F3 cells by shRNA (FIGS. 2D-F) or using AID-null splenic B cells (FIG. 1B) eliminated the AID ChIP signal from S [symbol] and the telomeres. Telomere occupancy by RNA polymerase II (RPII) and the transcription factor Spt5, which are necessary for recruiting AID to the DNA, and steady state levels of telomeric transcripts (TERR) did not change upon inducing CSR (FIGS. 2D-F), suggesting unaltered telomeric transcription. The inventors conclude that AID interacts with, and might deaminate, the telomeric DNA in B cells concomitantly with CSR.

Together these data revealed that the telomeric DNA is a target of AID in activated B-cells.

### EXAMPLE 3

### UNG Protects B Cells from AID-Dependent Telomeres Loss

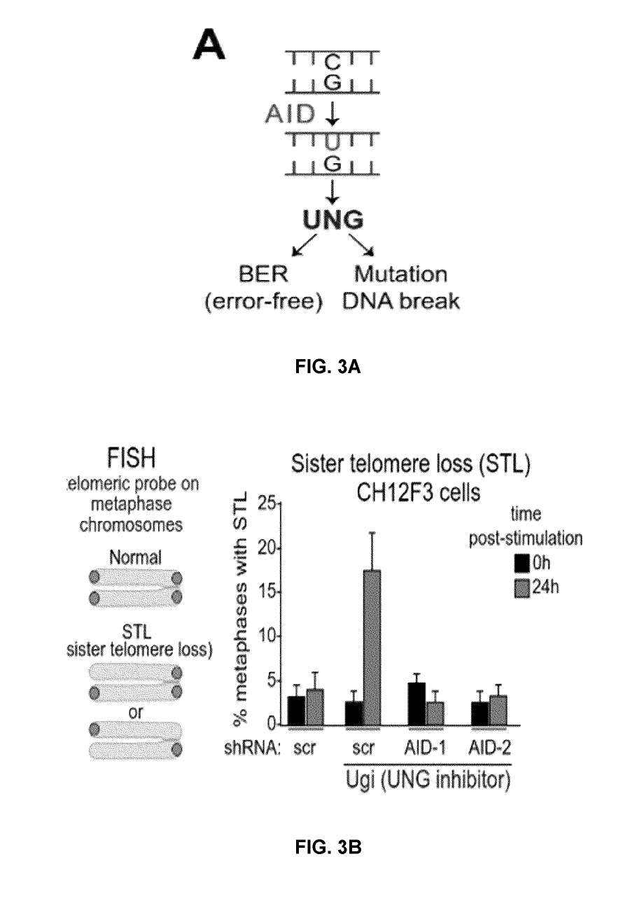

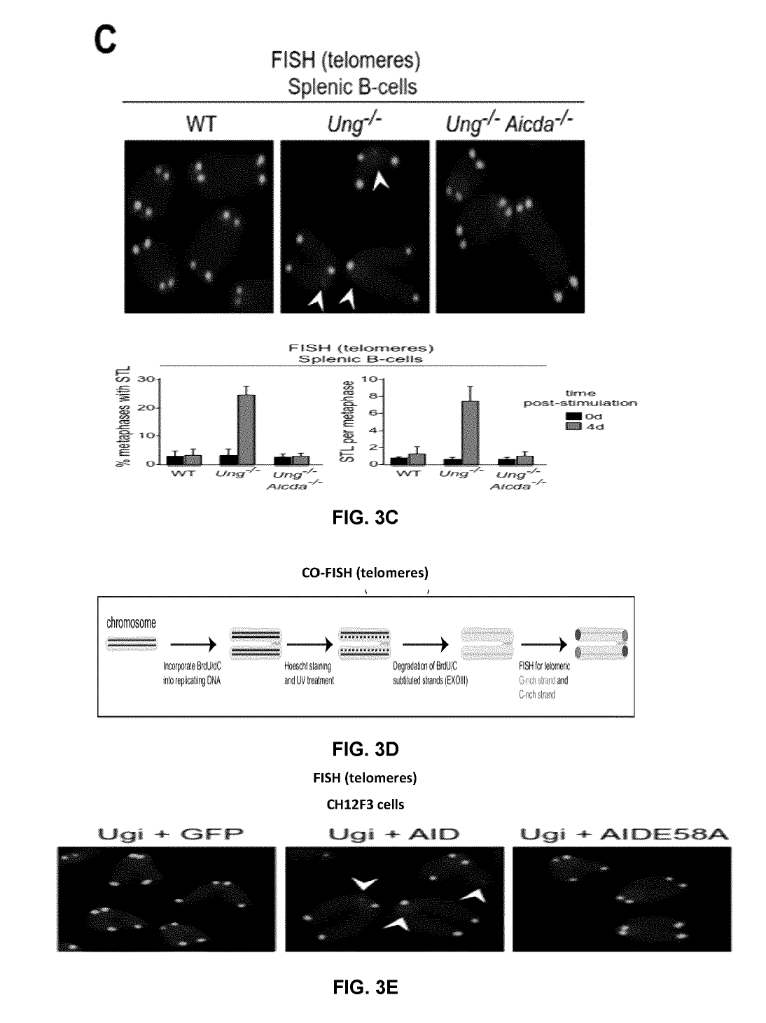

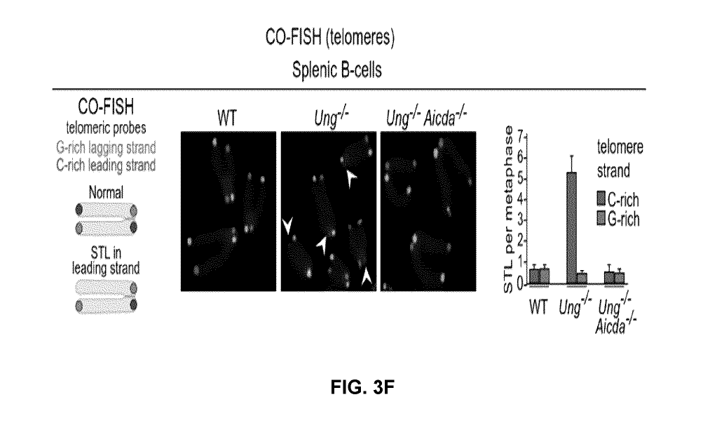

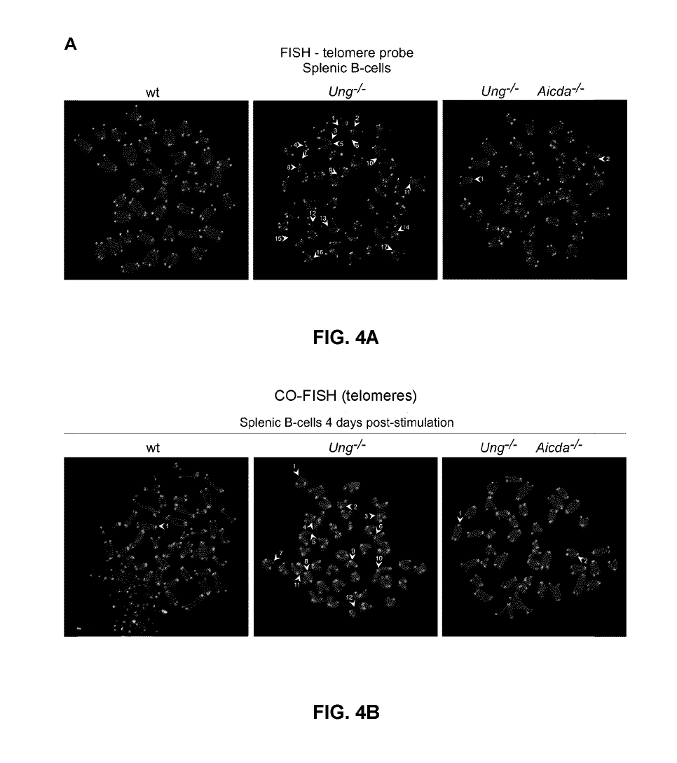

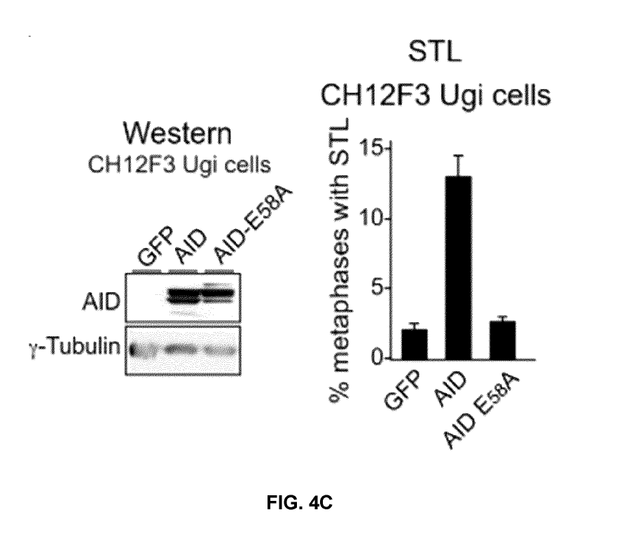

Because UNG-dependent BER is involved in the faithful as well as mutagenic processing of the DNA lesions induced by AID ^{32,20} the inventors wanted to determine whether the observed AID-dependent toxicity in UNG-null B-cells was associated with accumulation of unrepaired DNA breaks. To test this, the inventors analyzed the chromosome integrity of B-cells deficient in UNG before and after in vitro CSR stimulation. The inventors used fluorescent in situ hybridization (FISH) to label metaphase telomeres and visualize the chromosomes ends of WT and UNG−/−splenic B-cells stimulated in vitro for CSR to IgG1. Although activated UNG−/−B-cells did not show a significant accumulation of intra-chromatid breaks the inventors observed a significant loss of telomere signals when compared to WT B-cells (FIGS. 3B, C and 4A). Indeed, Ung−/−B-cells presented ˜8-fold increase in metaphases with lost telomere signals (24% Ung−/−vs 3% WT), and ˜5-fold increase in the number of telomere signal lost per metaphase when compared to WT B-cells (FIG. 3C). Further, loss of telomere signal was mostly at a single chromatid, resembling a phenotype known as sister telomere loss (STL) (FIG. 3B and ³³). Importantly, Ung−/−Aicda−/−B-cells showed a normal telomere staining pattern when compared to WT splenic B-cells (FIGS. 3C and 4A), revealing that the telomere loss observed in Ung−/−B-cells was AID-dependent. Although activated Ung−/−B-cells showed a clear loss of telomere signals the inventors did not observe an increase in telomere end-to-end fusions in Ung−/−B-cells when compared to WT B-cells (FIG. 4A), suggesting that telomeres were refractive to recombination events usually observed with dysfunctional or uncapped telomeres ³⁴.

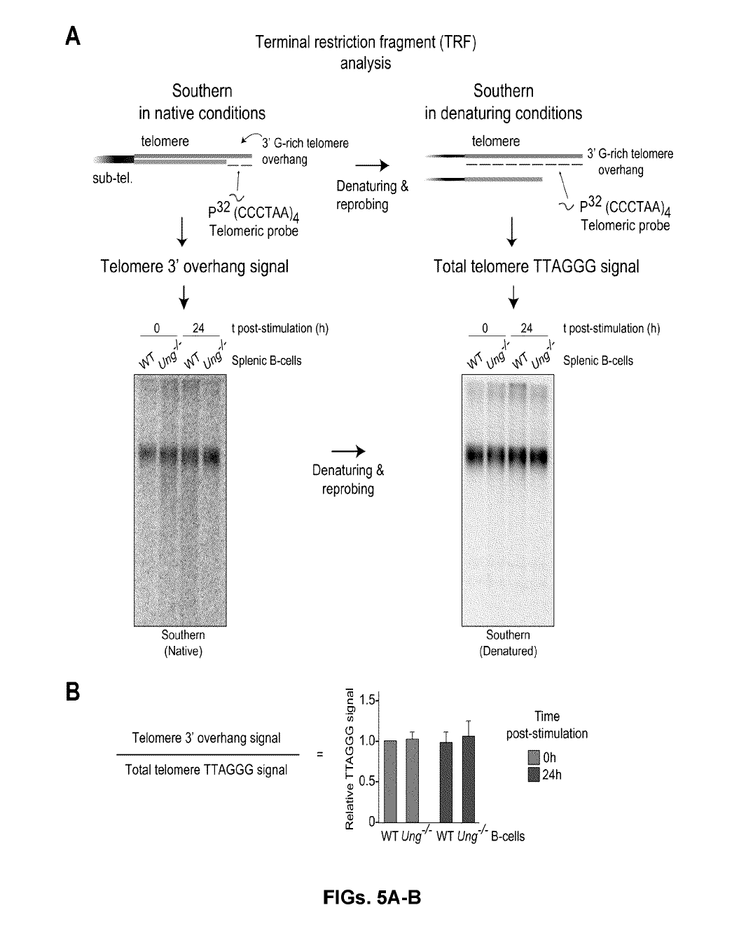

Also the inventors did not observe a change in the average telomere length as evaluated by terminal restriction fragment (TRF) Southern blot (FIGS. 5A-B) between activated Ung−/−and WT B-cells showing that the B-cells with sudden loss of telomeres do not stay in culture for too long. The inventors obtained similar results when studying activated CH12F3 B-cells. CH12F3 Ugi cells showed a significant increase in metaphase chromosomes with STL when compared with CH12F3 cells expressing GFP alone (FIG. 6A). Moreover, expression of human AID protein in CH12F3 Ugi cells produced a significant increase in STL when compared to control CH12F3 GFP cells, even without inducing class switching (FIG. 3E). However, expression of a deaminase-dead mutant AID (AIDE58A), showed a normal telomere staining even though the expression levels of both AID and AIDE58A were equivalent when analyzed via Western (FIGS. 3E and 4C). Based on these results the inventors conclude that AID's deaminase activity affects telomere stability in UNG-deficient B-cells. Since AID deaminates dC and that in the telomeres most, if not all, dC is present at the telomere strand replicated by the leading-strand replication machinery, the inventors wanted to assess whether UNG deficiency would preferentially affect one telomere strand (i.e. C-rich versus G-rich). The inventors used two-color chromosome orientation FISH (CO-FISH) ³⁵, in which bromo-deoxyuridine (BrdU) and bromo-deoxcytidine (BrdC) are incorporated into the freshly synthesized DNA strands. After degradation of the BrdU/dC containing DNA the template telomeric G-rich and C-rich strands can be discriminated by FISH using Alexa488-(CCCATT)₄ and Alexa594-(TTAGGG)₄ probes, respectively (FIG. 3D). Due to the strand-orientation in the telomeres CO-FISH yields two telomeric signals of each color per metaphase chromosome (i.e. green: telomeric G-rich strand (paler in FIG. 3D); red: telomeric C-rich strand (darker in FIG. 3D)). CO-FISH assays revealed that almost all STL in UNG-deficient B-cells were product of the replication of the C-rich telomeric strand (FIG. 3F). The normal CO-FISH staining observed in Ung−/−Aicda−/−splenic B-cells revealed that loss of C-rich telomeric DNA was AID-dependent (FIGS. 3F and 4B). The loss of telomeric DNA showed no clear preference in p- or q-arm (p/q STL ratio=0.91). Similar results were observed in CH12F3 cells expressing Ugi after stimulation for class switching to IgA (FIG. 6B). Of note, the inventors did not observe a significant difference in telomere sister chromatid exchange (T-SCE) in the splenic B-cells or CH12F3 cells analyzed (not shown), again suggesting that the damaged telomeres were refractive to recombination events. Altogether, these results show that UNG is essential to maintain the telomeres of B-cells expressing AID. The inventors conclude that AID deaminates telomeres in activated B cells, but the telomeres are efficiently protected from further DNA damage by UNG.

### EXAMPLE 4

### Knockdown of MSH2 prevents telomere loss in UNG-deficient B-cells

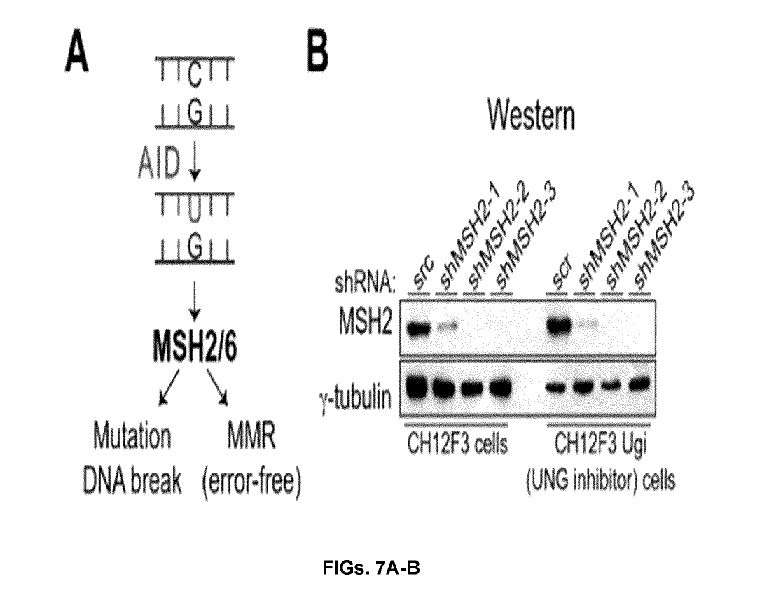

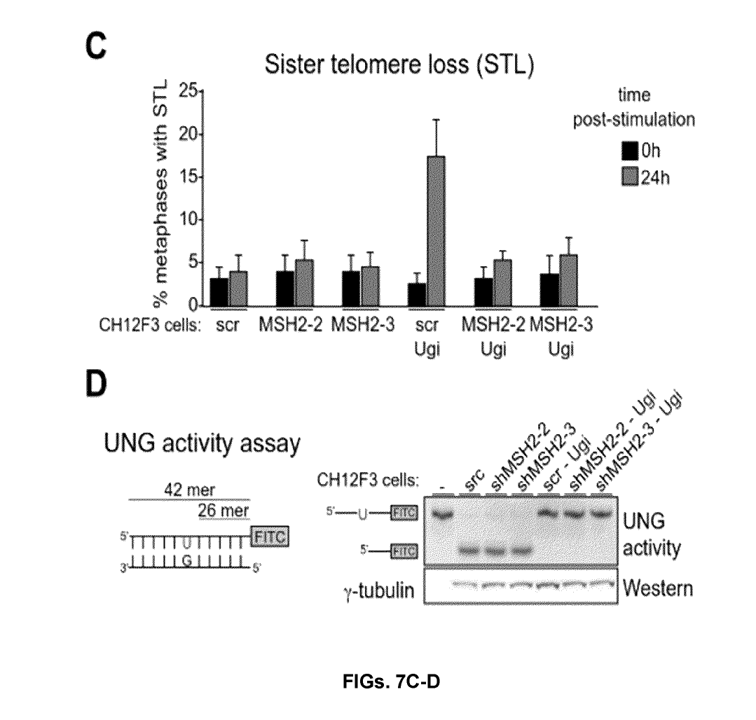

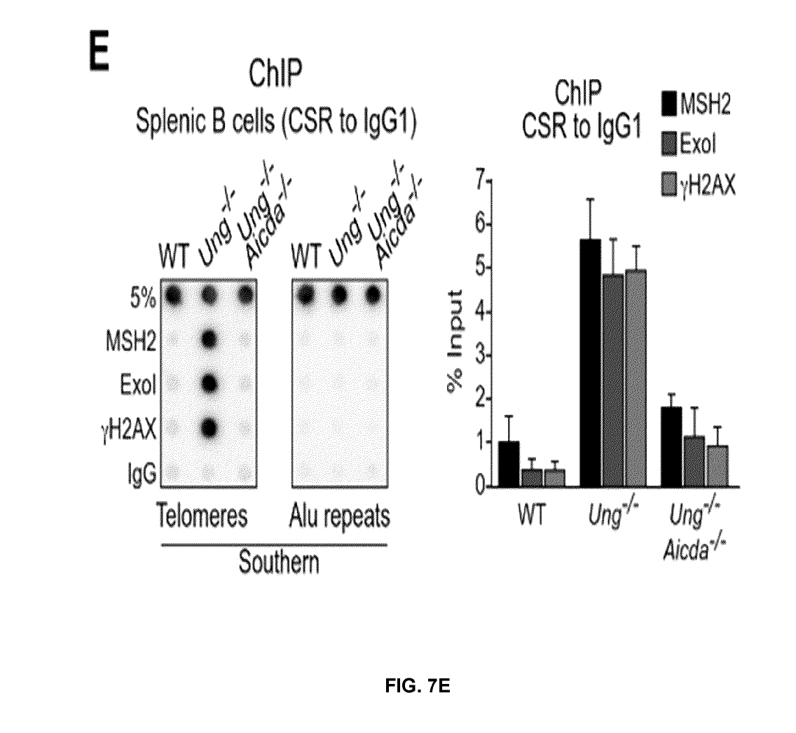

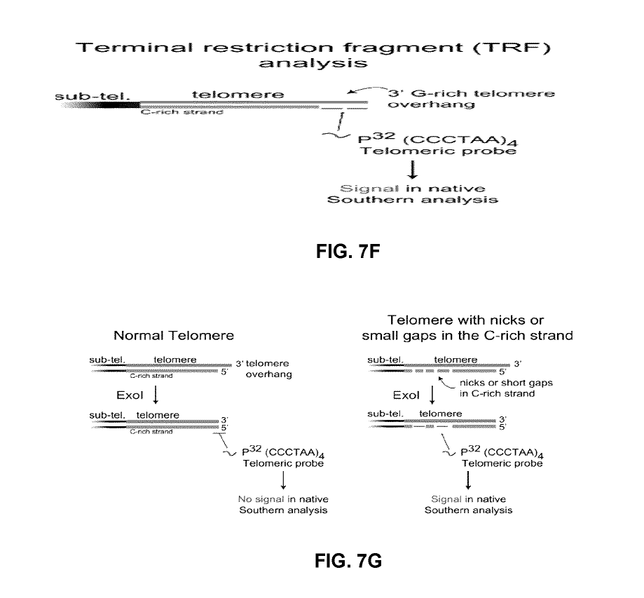

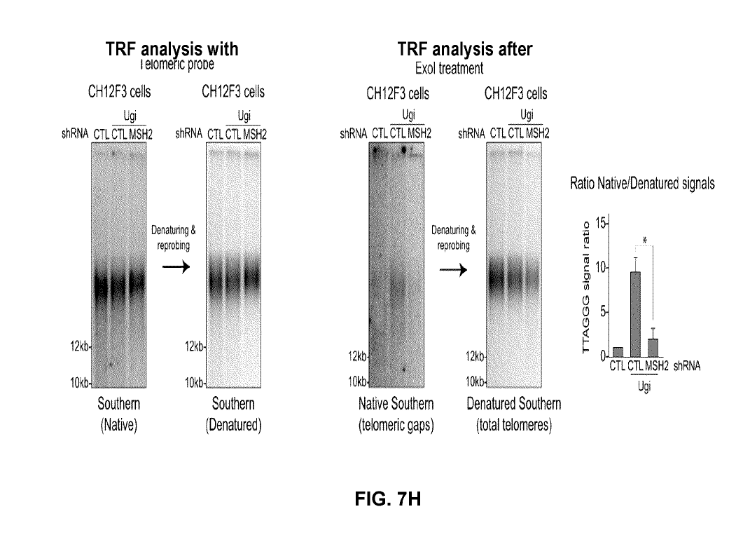

Although BER is the main mechanism used by B-cells for the processing of U produced by AID ², the G:U mismatches can also be processed by mismatch repair (MMR), for either error-prone or faithful repair ^{2,20,40}. Therefore, to determine whether MMR plays a similar role to BER in the protection of the telomeres during class switching the inventors knocked-down the MMR core factor MSH2 in CH12F3 and CH12F3 Ugi cells. FISH analysis of metaphase chromosomes from CH12F3 shMSH2 cells showed no increase in STL when compared to control cells (CH12F3 shGFP) before or after stimulation of CSR (FIG. 7C) Depleting MSH2 did not affect telomere stability in stimulated CH12F3 cells but prevented the increase in STL observed in CH12F3 Ugi cells, which lacked UNG activity (FIGS. 7B-D). These results suggested that MMR is not essential for maintenance of telomeres in activated WT B-cells. However, knockdown of MSH2 in UNG-deficient B-cells (CH12F3 Ugi shMSH2) showed STL values closer to control cells (CH12F3 GFP) and significantly lower than CH12F3 Ugi cells. Consistent with these findings, ChIP experiments showed MMR proteins MSH2 and EXO1 accumulation at the telomeres only in stimulated Ung−/−(FIG. 7E) and stimulated CH12F3 Ugi cells (data not shown). Importantly, ChIPs in Ung−/− Aicda−/− B-cells showed that the accumulation of MSH2 and EXO1 at the telomeres was AID-dependent (FIG. 7E). These results revealed that MMR mediates telomere loss in activated UNG-deficient B-cells but not in UNG-proficient B-cells suggesting that UNG normally outcompetes MMR for the processing of U induced by AID at the chromosome ends. Furthermore, although terminal restriction fragment analysis of CH12F3 Ugi cells showed that they had a normal telomere G-rich 3′ overhang signal (FIG. 7F), performing the same assay after treating the DNA with exonuclease to degrade this overhang revealed an MSH2-dependent increase in intra-telomeric G-rich single-stranded DNA (FIGS. 7G-H), indicative of ssDNA gaps.

The inventors conclude that, in the absence of UNG, MMR-dependent processing of AID lesions creates gaps in the telomeric C-rich strand thereby mediating STL in replicating B cells.

### EXAMPLE 5

### Short telomeres in UNG-deficient B-cells trigger a DNA damage response

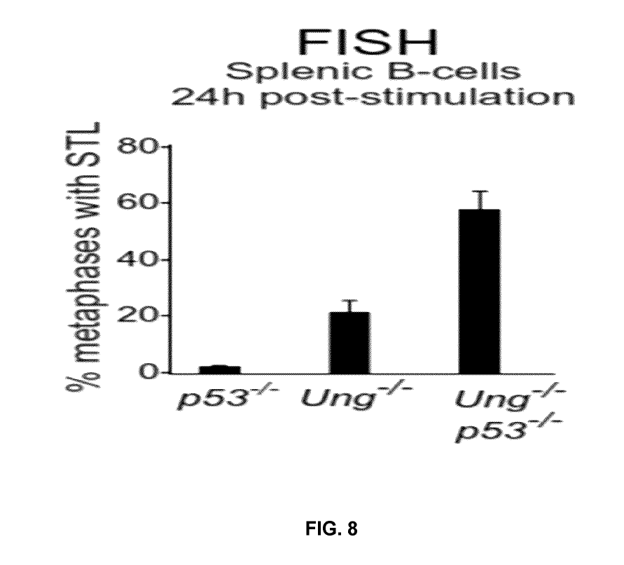

Excessive loss of telomeric DNA leads to induction of the DNA damage machinery at the chromosome ends ⁴¹⁻⁴³. Indeed, the inventors detected AID-dependent accumulation of phospho-ser129-H2AX (a marker of DNA damage often found in dysfunctional telomeres in stimulated Ung^{-/-} B cells ⁴²), at the telomeres of UNG-deficient B-cells (CH12F3 Ugi) when compared with control B-cells (CH12F3 GFP) (FIG. 7E). The accumulation of ser129-H2AX was AID dependent as revealed by the lack of ser129-H2AX signal in the extracts from CH12F3 Ugi shAID cells (FIG. 7E). A DNA damage response at the telomeres generally triggers a p53-dependent cell cycle arrest ⁴²⁻⁴⁶. To establish whether the observed loss of telomeric DNA in activated UNG-deficient B-cells leads to a p53-dependent arrest, the inventors analyzed the frequency of STL in splenic Ung−/−and p53−/−Ung−/− splenic B-cells stimulated for class switching. As predicted, the number of STL was sharply elevated in p53−/−Ung−/− splenic B-cells when compared to Ung−/−splenic B-cells (FIG. 8), indicating that the AID-dependent telomere loss in activated UNG-deficient B-cells triggers a cell division arrest or apoptosis.

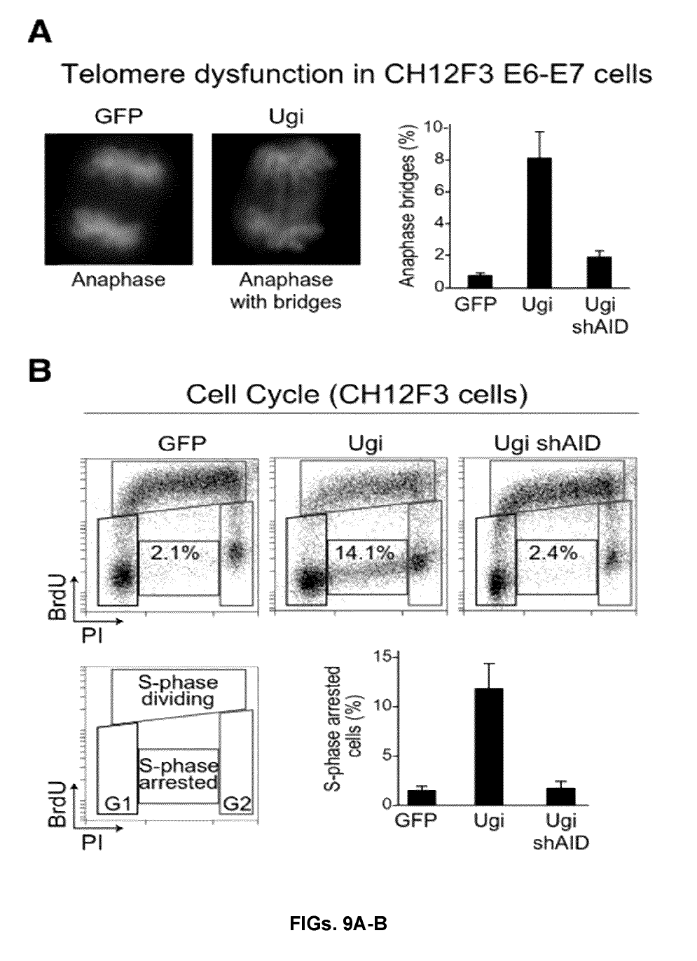

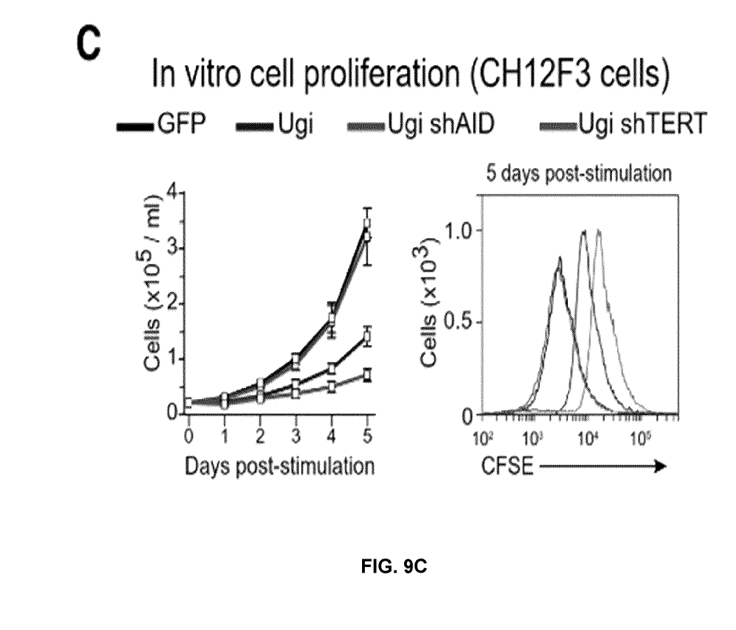

By suppressing the p53 and p16INK4a/pRb-dependent pathways via the expression of papillomavirus proteins E6 and E7 to prevent B cell death, the inventors found that CH12F3 Ugi cells significantly accumulated anaphase bridges compared to CH12F3 Ugi shAID and control GFP cells (FIG. 9A). Thus, AID expression causes telomere dysfunction in UNG-deficient B cells.

A DNA damage response due to telomere dysfunction usually causes cell division defects. Accordingly, cell cycle profiling revealed that stimulated UNG-deficient B cells had a ˜6-fold increase in cells arrested in S-phase, which was AID-dependent (FIG. 9B). These results predicted that UNG-deficient B cells expressing AID should have a limited proliferation capacity. Indeed, CH12F3 Ugi cells stimulated for CSR showed reduced growth, as evaluated by total cell number or CFSE dilution assay, which was prevented by AID knockdown (FIG. 9C). In addition, consistent with the role of telomerase in protecting against cell proliferation defects caused by excessive telomere shortening, knockdown of the catalytic subunit of telomerase in CH12F3 Ugi cells further decreased their proliferation (FIG. 9C).

The inventors conclude that in the absence of UNG, AID induces telomere dysfunction and a DNA damage response that compromises B cell proliferation.

### EXAMPLE 6

### Compromised Germinal Center B Cell Expansion in Ung^{-/-} Mice

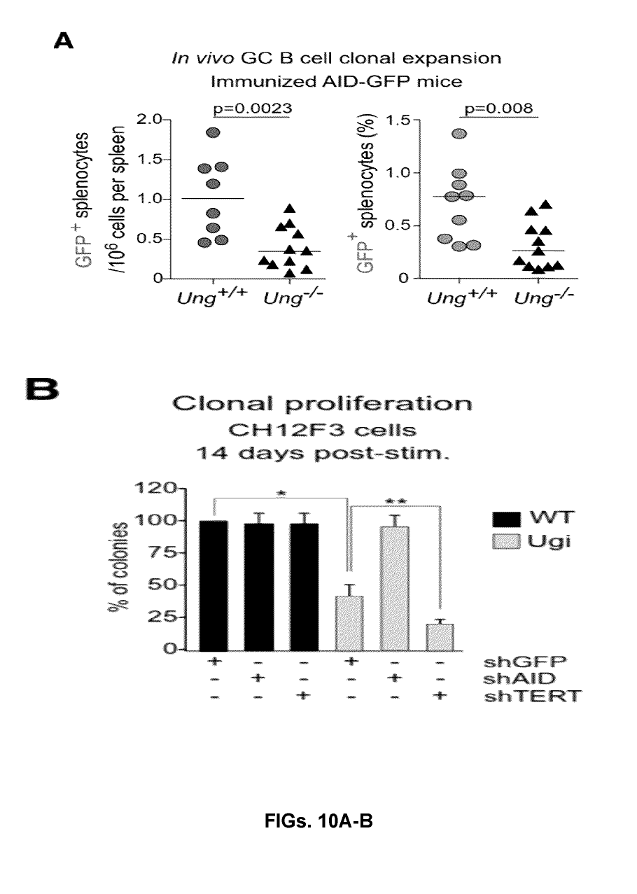

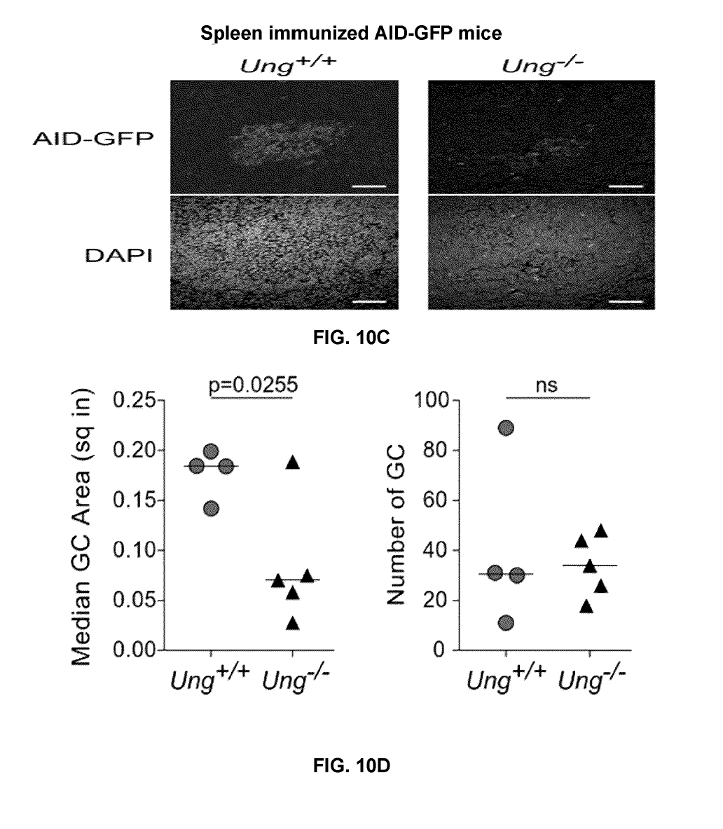

B cells must expand clonally while expressing AID during the GC reaction. Based on the results described above the inventors asked whether UNG deficiency might limit the proliferation capacity of transformed B-cells expressing AID. To assess the effect of UNG deletion on the proliferation of B-cells expressing AID, the inventors immunized WT and UNG-null mice with a T-dependent antigen to stimulate germinal center (GC) B-cell expansion and AID expression in vivo. The inventors used an AID-GFPtg reporter mouse that allows identifying AID+ GC B-cells in response to immunization ^{6,28}. Seven days post-immunization, Ung−/−AID-GFPtg mice showed a ˜50% fewer splenic AID+(i.e. GC) B-cells than control AID-GFPtg mice (FIG. 10A). This reduction was explained by the ˜3-fold smaller average GC size in Ung^{-/-} versus Ung^{+/+} AID-GFPtg mice, rather than any difference in the number of GC (FIGS. 10C-D). As previously shown for purified splenic Ung−/−B-cells ²⁹, AID-GFPtg Ung−/−B-cells did not show reduced proliferation in vitro compared to AID-GFPtg B-cells after stimulation with LPS and IL-4, as judged the number of live cells (not shown). However, it must be noted that at variance with immunization, which stimulates a few B-cells that need to undergo clonal expansion to form the GC, stimulation in vitro with cytokines causes massive proliferation accompanied by a high level of spontaneous cell death, which likely obscures the additional increase due to UNG-deficiency (not shown). To address this limitation, the inventors evaluated the cloning efficiency of single B-cells. The inventors used the CH12F3-2, a mouse lymphoma cell line that induces AID and undergoes class switching to IgA in response to stimulation with transforming growth factor b (TGFb), CD40 ligation, and IL-4 ³⁰. Hence, CH12F3-2 cells expressing GFP control (CH12F3 GFP) or expressing the bacteriophage PSB-2 gene encoding the UNG inhibitor Ugi ³¹ were grown during 14 days after single-cell dilution in presence of cytokines to induce CSR to IgA (FIG. 10B). In these assays the inventors observed a significant decrease in the cloning capacity of CH12F3-2 Ugi cells when compared to control cells (CH12F3-2 GFP). This proliferation defect was AID-dependent as revealed by the CH12F3-2 Ugi shAID cells presenting proliferation values similar to control cells (FIG. 10B). Altogether, these results strongly suggest that UNG deficiency substantially impairs proliferation of B-cells expressing AID in vitro and in vivo.

### EXAMPLE 7

### UNG deficiency Leads to proliferation Defects in AID+ B-cell lymphomas

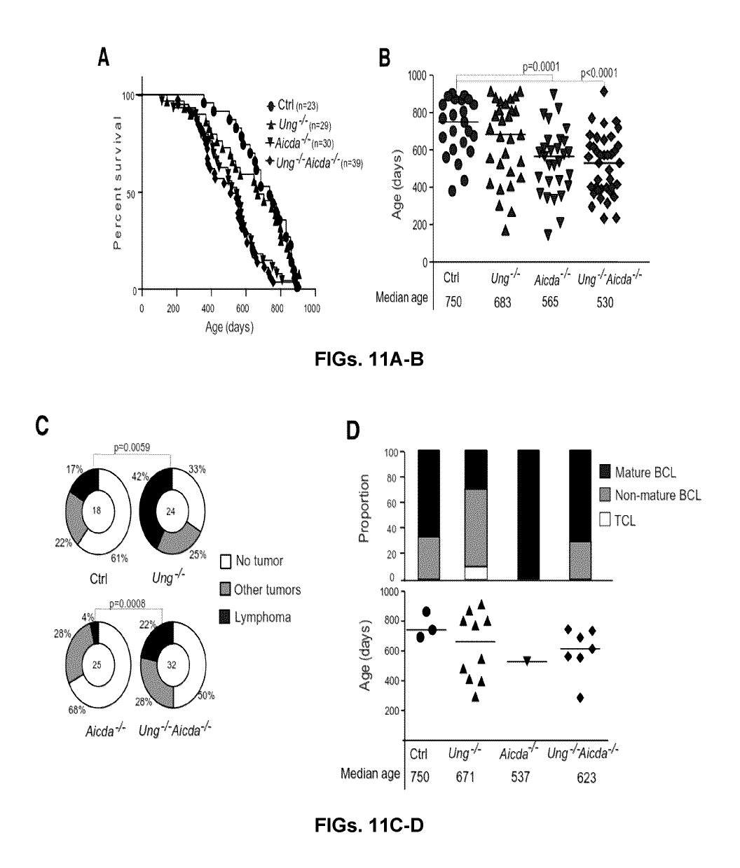

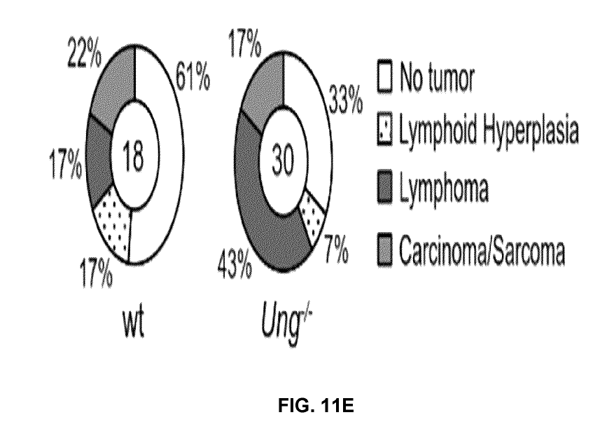

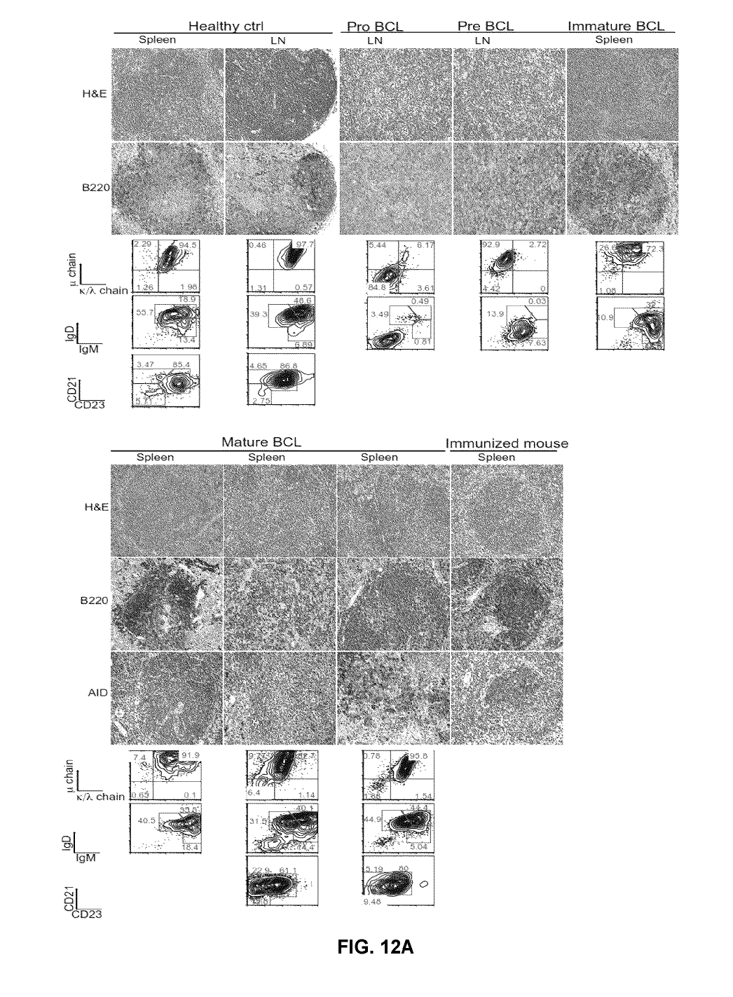

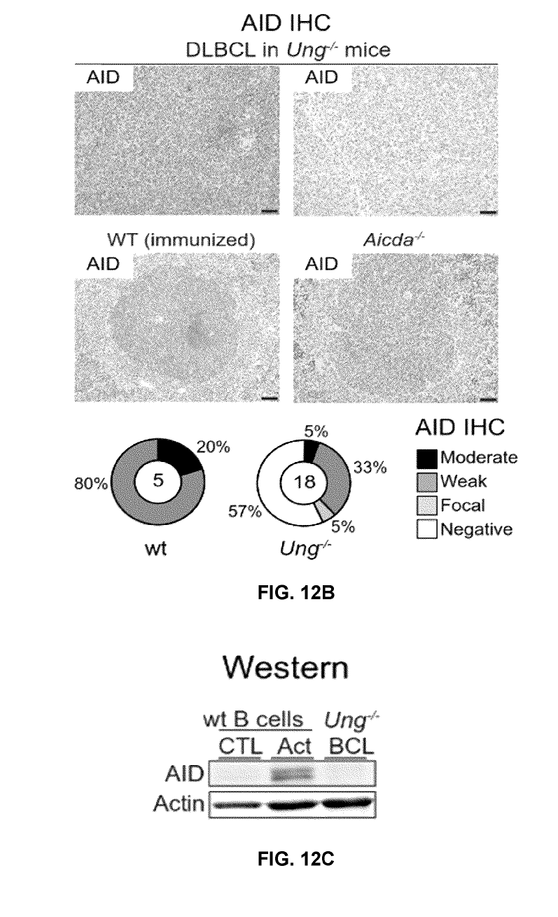

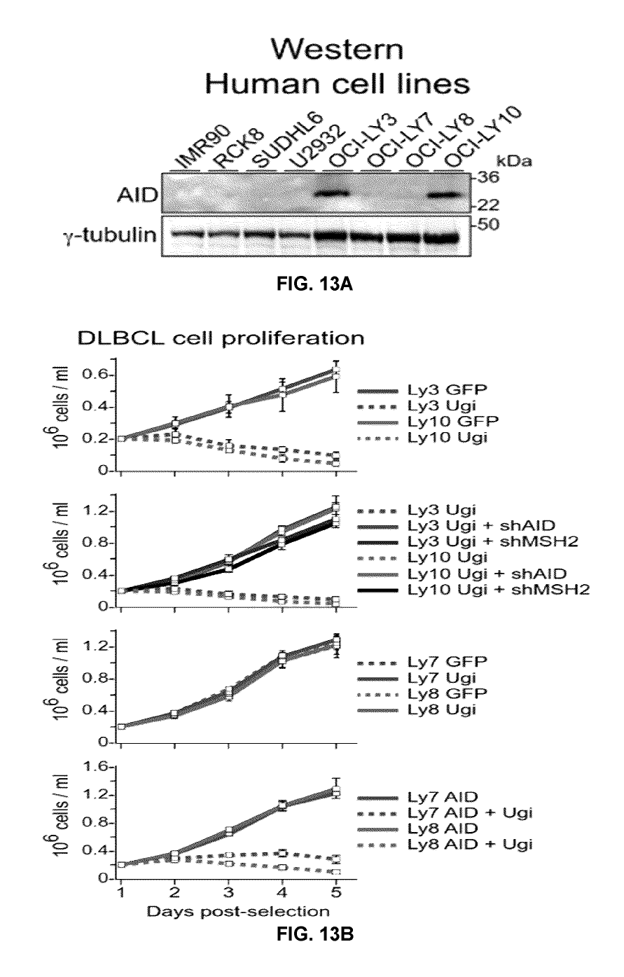

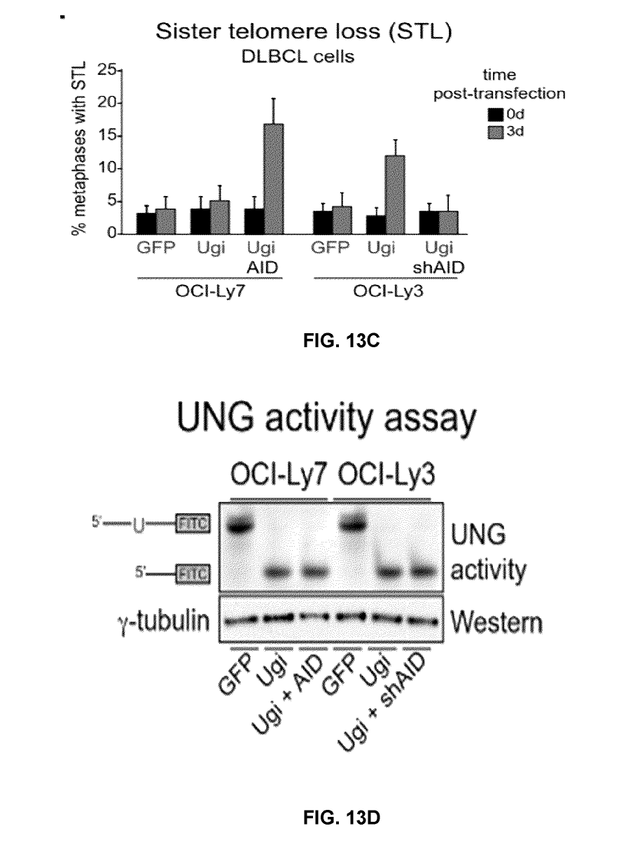

Old Ung-deficient mice are prone to develop B cell lymphomas ²². Although it has been speculated that AID might be etiological in those tumors, the AID status of the actual lymphomas has not been studied. The inventors analyzed lymphomas that developed spontaneously in wt and Ung^{-/-} mice. The median survival time of Ung^{-/-} mice was ˜10 weeks shorter tin median survival than wt (FIGS. 11A, B). 13 out of 30 Ung^{-/-} mice (versus only 3 of 18 control mice) developed lymphoma (FIG. 11E) which in most cases had a histopathology consistent with mature B cell lymphoma (FIG. 13 and Table V below). This difference was not significant for the used cohorts of 20-30 mice, but is very similar to the significant 13 weeks reduction previously reported for a cohort of >100 mice ²². Further, and as previously described ^{22,23}, the inventors observed a significant increase in the incidence of lymphoma in Ung−/− mice, which were mostly but not exclusively B-cell lymphomas (FIGS. 11C, D). Interestingly, detailed phenotyping of the lymphomas in Ung−/−mice by flow cytometry showed that a majority of them were progenitor or immature B-cell lymphomas (Non-mature BCL) (FIGS. 11D and 12D), which are B-cell stages that do not express AID. In fact, AID expression was detected in only one of the three Ung−/−mice that developed mature BCL, and in none of the progenitor or immature, B-cell lymphomas; as shown by IHC (FIG. 2 and Table V). The majority (57%) of Ung^{-/-} lymphomas were negative for AID by IHC (FIGS. 12B, 13 and Table V and Western analysis (FIG. 12C). The low or negative expression of AID in Ung^{-/-} B cell lymphomas is consistent with the notion that high AID expression is not well tolerated by UNG-deficient B cells.

TABLE V
Phenotype of spontaneous lymphomas in WT, Ung-/-, Aicda-/- and Ung-/- Aicda-/- mice

| | Mouse | | | Flow cytometry | | | | | IHC | | | |
|---|---|---|---|---|---|---|---|---|---|---|---|---|
| | | Age | Enlarged | Analyzed | | | κ/ | | | B22 | | |
| | ID | (d) | organs | organs | B220 | μ | λ | IgD | CD3 | 0 | AID | Pathology observations |
| WT | 1484 | 872 | LN (Ms), | Mes LN | + | − | + | − | − | + | Weak | Diffuse large cell lymphoma |
| | | | Li | | | | | | | | | |
| | 19-135 | 888 | LN (Mes), | Mes LN | + | − | − | − | − | + | Weak | Diffuse large cell lymphoma |
| | (1) | | Li | Spleen | + | + | − | − | − | + | Weak | Large cell lymphoma, nodular pattern |
| | 1517 | 750 | LN (Ms, RI) | Spleen | + | − | − | − | − | + | Moderate | Multiple Nodular lymphoid expansion |
| | | | | Mes LN | + | − | − | − | − | + | Weak | Diffuse large cell lymphoma |
| Ung^{-/-} | 1076 | 874 | | Spleen | + | + | + | + | − | + | − | Expanded lymphoid nodules, some confluents |
| | | | | | | | | | | | | with large atypical lymphoid cells and mitoses |
| | | | | | | | | | | | | consistent with large B cell lymphoma |
| | 1175 | 923 | LN (Md) | Med LN | ND | ND | N | ND | − | + | Weak | Diffuse large cell lymphoma morphology |
| | | | | | | | D | | | | | |
| | 1445 | 813 | LN (Ms, | Mes LN | ND | ND | N | ND | − | + | − | Diffuse large cell morphology. Numerous |
| | | | Md), Li | | | | D | | | | | mitoses. B220 weakly positive |
| | | | | Liver | ND | ND | N | ND | − | + | Focally | Focal, intrasinusoidal clusters. |
| | | | | | | | D | | | | | |
| | 1645 | 783 | LN (Md, | Spleen | U | U* | U* | U | − | + | − | Nodular pattern, large lymphoid cells |
| | | | Ms, Lu) | | | | | | | | | positive for B220 |
| | | | | Med LN | + | + | + | + | − | + | − | Diffuse and vaguely nodular, large cell |
| | | | | | | | | | | | | morphology, some plasmablastic-like. |
| | | | | | | | | | | | | Numerous mitoses. B220 weakly positive |
| | 1237 | 494 | LN (Su, | Spleen | + | + | + | − | − | + | − | Lymphoid hyperplasia, nodular pattern, |
| | | | Ax, Ig, Lu, | | | | | | | | | intermediate to large lymphoid cells |
| | | | Md) | | | | | | | | | |
| | 1151 | 881 | LN (Ms, | Med LN | + | − | − | − | − | + | − | Diffuse large cell lymphoma |
| | | | Md) | | | | | | | | | |
| | 1300 | 814 | LN (Ms), | Mes LN | + | + | − | − | − | | Weak | Diffuse large cell lymphoma |
| | | | Li | | | | | | | | | |
| | 1664 | 559 | Sp, LN | Spleen | + | + | − | − | − | + | − | Nodular pattern, similar to follicular |
| | | | (Ms, Md, | | | | | | | | | lymphoma |
| | | | Su, Lu) | | | | | | | | | |
| | 2182 | 809 | Sp, LN | Spleen | + | + | − | − | − | + | Weak | Large cell lymphoma, nodular |
| | | | (Ms, Md, | | | | | | | | | and diffuse patterns |
| | | | Su, Ax, | Mes LN | + | + | − | − | | | − | Diffuse large cell lymphoma, |
| | | | Ig), Li | | | | | | | | | numerous plasma cells are also seen |
| | | | | Med LN | + | + | − | − | | | − | Diffuse large cell lymphoma, numerous |
| | | | | | | | | | | | | plasma cells are also seen |

L, localized, D, disseminated (≥2 locations)

These results may appear contradictory to previous works that reported that B-cell lymphomas Ung−/−0 mice were of the FL or DLBCL type ^{22,23}. However, the inventors note that in those works, lymphomas were analyzed only by histology and not through detailed surface marker phenotyping, as the inventors have performed. The inventors could not compare survival of Ung−/− versus Ung−/− Aicda−/− mice because AID-deficiency alone resulted in reduced lifespan (FIG. 11A). The reason why AID-deficiency reduced median survival was not obvious or explored further but it is likely due autoimmune complications ²⁴⁻²⁶, although the inventors note that the Lag-3 mutation linked to the original Aicda knock-out ²⁷ was not present in the cohorts used herein (not shown). In any case, Ung−/− Aicda−/− mice still showed significantly higher incidence of B-cell lymphoma compared to Aicda−/− mice (FIGS. 11C, D). In this case most lymphomas were of mature B-cell type (FIGS. 11D and 12). These results confirmed that UNG-deficiency indeed increases the incidence of spontaneous B-cell lymphomas in mice, but this effect is AID-independent.

### EXAMPLE 8

### UNG deficiency affects the proliferation capacity of DLBCL cells expressing AID

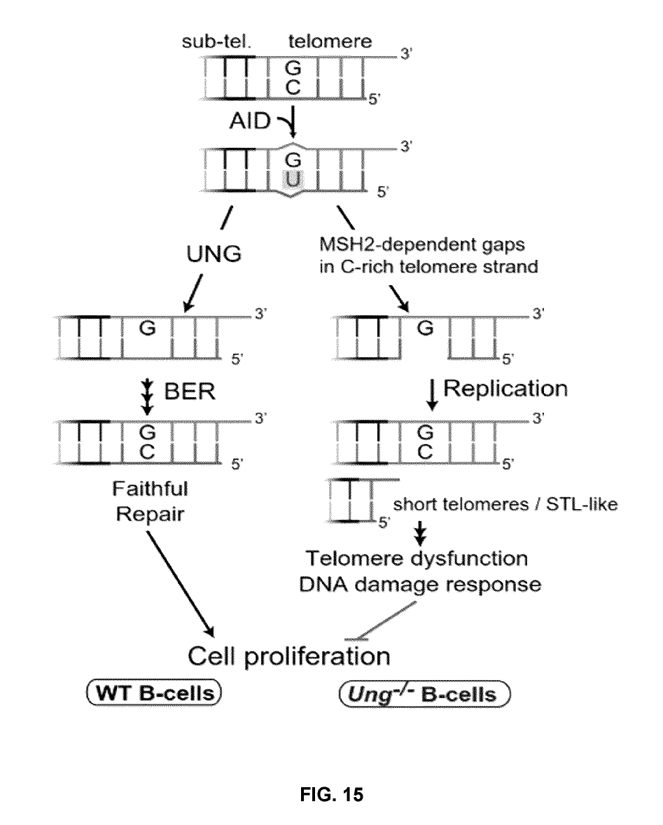

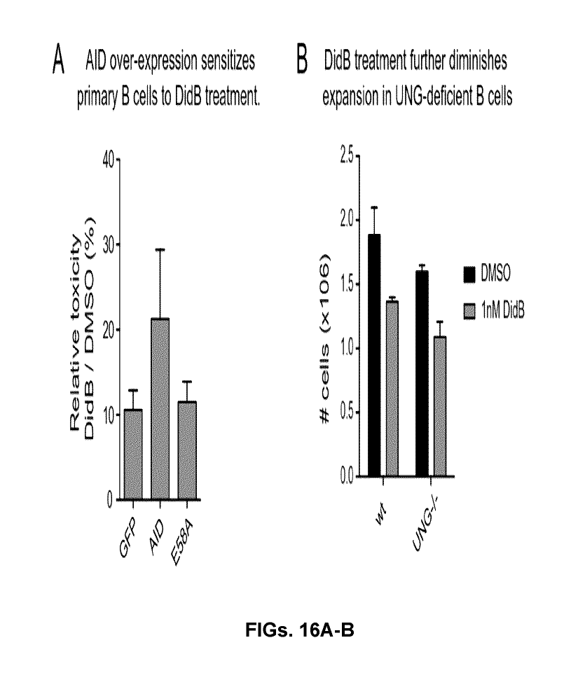

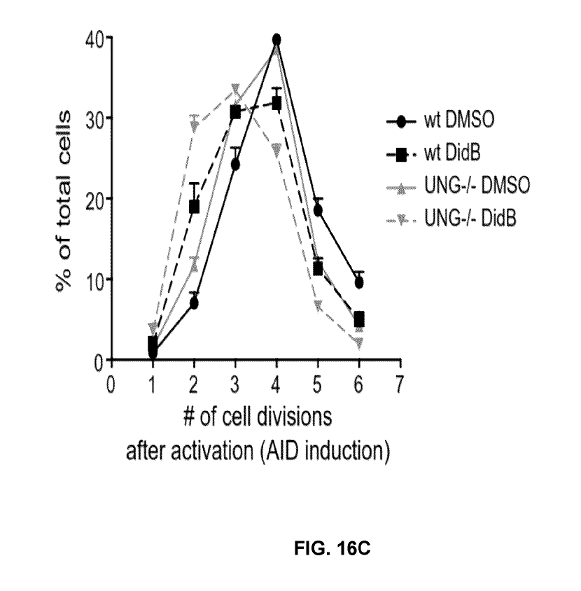

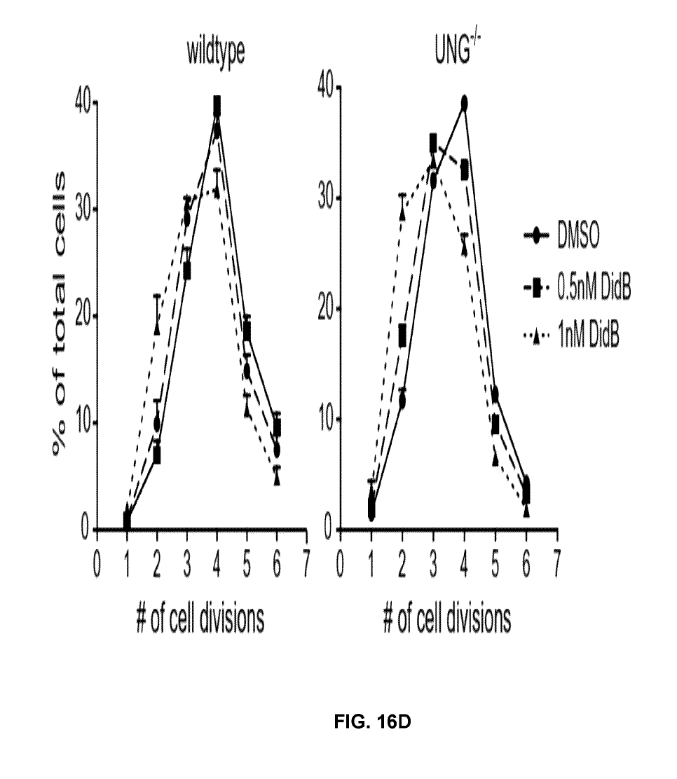

In contrast to GC B Bells in which AID is acutely induced human non-Hodgkin's lymphoma (NHL) cells such as diffuse large B cell lymphoma (DLBCL) can steady express of AID to various levels ^{8,9}. The inventors therefore stratified human DLBCL cell lines on the basis of their AID protein levels into high (DLBCL AID⁺) (Ocy-Ly3 and Ocy-Ly10 in FIG. 13A) or low expressing (DLBCL AID⁻) lines (Ocy-Ly7 and Ocy-Ly8 in FIG. 13A) and established derivatives expressing Ugi for each one. DLBCL AID+, cells showed a lower rate of cell proliferation when compared to DLBCL AID−. (FIG. 13B) Although DLBCL AID+ cells showed a lower rate of cell proliferation when compared to DLBCL AID− cells (FIG. 13B), Ugi only impaired the growth of DLBCL AID+ cells (FIG. 12B). The poor growth and sensitivity to Ugi of DLBCL AID+ cells was both AID− and MMR-dependent, as knockdown of AID or MSH2 allowed their proliferation at levels comparable to DLBCL AID⁻ cells (FIG. 13B. Furthermore, DLBCL AID⁻ cells became sensitive to UNG inhibition after transfection with AID (FIG. 13B). Importantly, the inventors observed a significant increase in the number of metaphases with STL only when DLBCL cells expressed AID and Ugi but not with Ugi alone (FIG. 13C), despite having a similar level of UNG inhibition (FIG. 13D). These results revealed that human B-cell lymphoma cells expressing AID depend on UNG activity for maintenance of telomeres and their proliferation capacity and points to MMR as a main player on this UNG-dependent proliferation defect (FIG. 15).

### EXAMPLE 9

### Synthesis of UNG inhibitors

The synthesis of UNG inhibitors useful for the present invention is described in Krosky et al.⁶⁴ and in WO2006135763. Briefly, solutions (0.15 M) of 4-carboxybenzaldehyde (888 μl, 0.165 mmol), 6-formyluracil (888 μl, 0.165 mmol) and acetic acid (888 μl, 0.165 mmol) in DMSO are added to a reaction vessel. The reaction is initiated by the addition of 0.15 M O,O′-diaminoethanediol (888 ′l, 0.165 mmol) in DMSO, and incubated at 37° C. for 36 h. The desired heterosubstituted compound 1 is purified by direct injection of the reaction mixture onto a Phenomenex Aqua reversed phase C-18 HPLC column (250 mm, 10 mm, 5 μm) using gradient elution from 0 to 65% CH₃CN in 0.1 M aqueous TEAA over the course of 2 h using UV detection at 320 nm. Fractions containing the resulting compound are combined and concentrated in vacuo. The compound is precipitated using ice-cold water, centrifuged, washed twice with ice-cold water and dried in vacuo. This yields a compound as a white powder.

Another useful compound (see compound 2 below) is synthesized and purified as described above except that 0.15 M solutions of 3-carboxybenzaldehyde and O,O′-diaminopropanediol are used.

### EXAMPLE 10

### Effect of combination of UNG deficiency with EEF1A inhibition on AID toxicity in primary B cells

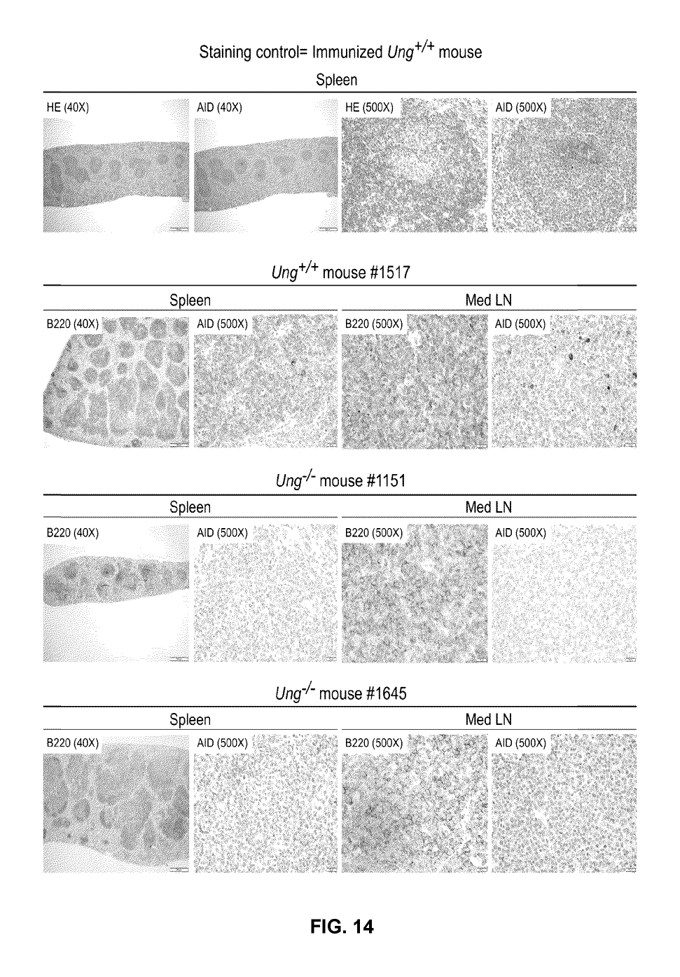

Primary B cells were harvested from AID UNG double deficient mice and cultured with anti-CD180, IL-4 and LPS. 24 h later cells were infected with either GFP alone, AID-ires-GFP or AIDE58A-ires-GFP (a catalytically inactive AID variant). 24 h post infection, cells were treated with either DMSO vehicle or 1 nM of the EEF1A inhibitor DidB. Toxicity was measured after 72 h as the % difference of GFP+ cells in the DidB-treated culture compared to DMSO control. Data from two mice are averaged, bars are SEM. The experiment shows that UNG-deficient B cells are substantially more sensitive to DidB when AID is expressed. Results are shown in FIG. 14A.

B cells were purified from wt or UNG−/− mice, and cultured directly with LPS and IL-4 and were counted 48 h post-treatment in order to measure B cell expansion (FIG. 14B); or after loading with the CFSE dye and cell divisions for treated cells were measured by analyzing the dilution of CFSE dye at 24 h post-treatment. Dilution of CFSE in cells is proportional to proliferation (FIGS. 14C-D).

24 h later, cells were treated with either DMSO or DidB. Results are shown in FIGS. 14B-D. Result show that DidB treatment further diminishes expansion in Ung-deficient B cells (FIG. 14B); that UNG-deficient B cells are more sentitive to DidB treatment than wt B cells (FIG. 14C); and that dose-dependent inhibition of proliferation is more marked in Ung-deficient B cells than wt B cells.

### REFERENCES

1 MacLennan, I. C. Germinal centers. Annu Rev Immunol 12, 117-139, (1994). 2 Stavnezer, J., Guikema, J. E. & Schrader, C. E. Mechanism and regulation of class switch recombination. Annu Rev Immunol 26, 261-292, (2008).

3 Nambu, Y., Sugai, M., Gonda, H., Lee, C. G., Katakai, T., Agata, Y., Yokota, Y. & Shimizu, A. Transcription-coupled events associating with immunoglobulin switch region chromatin. Science 302, 2137-2140, (2003).

4 Pavri, R., Gazumyan, A., Jankovic, M., Di Virgilio, M., Klein, I., Ansarah-Sobrinho, C., Resch, W., Yamane, A., Reina San-Martin, B., Barreto, V., Nieland, T. J., Root, D. E., Casellas, R. & Nussenzweig, M. C. Activation-induced cytidine deaminase targets DNA at sites of RNA polymerase II stalling by interaction with Spt5. Cell 143, 122-133,(2010).

5 Basu, U., Meng, F. L., Keim, C., Grinstein, V., Pefanis, E., Eccleston, J., Zhang, T., Myers, D., Wasserman, C. R., Wesemann, D. R., Januszyk, K., Gregory, R. I., Deng, H., Lima, C. D. & Alt, F. W. The RNA exosome targets the AID cytidine deaminase to both strands of transcribed duplex DNA substrates. Cell 144, 353-363, (2011).

6 Crouch, E. E., Li, Z., Takizawa, M., Fichtner-Feigl, S., Gourzi, P., Montano, C., Feigenbaum, L., Wilson, P., Janz, S., Papavasiliou, F. N. & Casellas, R. Regulation of AID expression in the immune response. J Exp Med 204, 1145-1156, (2007).

7 Orthwein, A. & Di Noia, J. M. Activation induced deaminase: how much and where? Semin Immunol 24, 246-254, (2012).

8 Lossos, I. S., Levy, R. & Alizadeh, A. A. AID is expressed in germinal center B-cell-like and activated B-cell-like diffuse large-cell lymphomas and is not correlated with intraclonal heterogeneity. Leukemia 18, 1775-1779, (2004).

9 Pasqualucci, L., Guglielmino, R., Houldsworth, J., Mohr, J., Aoufouchi, S., Polakiewicz,R., Chaganti, R. S. & Dalla-Favera, R. Expression of the AID protein in normal and neoplastic B-cells . Blood 104, 3318-3325, (2004).

10 Daniel, J. A. & Nussenzweig, A. The AID-induced DNA damage response in chromatin. Mol Cell 50, 309-321, (2013).

11 Manis, J. P., Tian, M. & Alt, F. W. Mechanism and control of class-switch recombination. Trends Immunol 23, 31-39, (2002).

12 Alt, F. W., Zhang, Y., Meng, F. L., Guo, C. & Schwer, B. Mechanisms of programmed DNA lesions and genomic instability in the immune system. Cell 152, 417-429, (2013).

13 Bemark, M. & Neuberger, M. S. The c-MYC allele that is translocated into the IgH locus undergoes constitutive hypermutation in a Burkitt's lymphoma line. Oncogene 19, 3404-3410, (2000).

14 Muschen, M., Re, D., Jungnickel, B., Diehl, V., Rajewsky, K. & Kuppers, R. Somatic mutation of the CD95 gene in human B-cells as a side-effect of the germinal center reaction. J Exp Med 192, 1833-1840, (2000).

15 Pasqualucci, L., Migliazza, A., Fracchiolla, N., William, C., Neri, A., Baldini, L., Chaganti, R. S., Klein, U., Kuppers, R., Rajewsky, K. & Dalla-Favera, R. BCL-6 mutations in normal germinal center B-cells : evidence of somatic hypermutation acting outside Ig loci. Proc Natl Acad Sci U S A 95, 11816-11821, (1998).

16 Pasqualucci, L., Neumeister, P., Goossens, T., Nanjangud, G., Chaganti, R. S., Kuppers, R. & Dalla-Favera, R. Hypermutation of multiple proto-oncogenes in B-cell diffuse large-cell lymphomas. Nature 412, 341-346, (2001).

17 Shen, H. M., Peters, A., Baron, B., Zhu, X. & Storb, U. Mutation of BCL-6 gene in normal B-cells by the process of somatic hypermutation of Ig genes. Science 280, 1750-1752, (1998).

18 Qian, J., Wang, Q., Dose, M., Pruett, N., Kieffer-Kwon, K. R., Resch, W., Liang, G., Tang, Z., Mathe, E., Benner, C., Dubois, W., Nelson, S., Vian, L., Oliveira, T. Y., Jankovic, M., Hakim, 0., Gazumyan, A., Pavri, R., Awasthi, P., Song, B., Liu, G., Chen, L., Zhu, S., Feigenbaum, L., Staudt, L., Murre, C., Ruan, Y., Robbiani, D. F., Pan Hammarstrom, Q., Nussenzweig, M. C. & Casellas, R. B-cell super-enhancers and regulatory clusters recruit AID tumorigenic activity. Cell 159, 1524-1537, (2014).

19 Meng, F. L., Du, Z., Federation, A., Hu, J., Wang, Q., Kieffer-Kwon, K. R., Meyers, R. M., Amor, C., Wasserman, C. R., Neuberg, D., Casellas, R., Nussenzweig, M. C., Bradner, J. E., Liu, X. S. & Alt, F. W. Convergent transcription at intragenic super-enhancers targets AID-initiated genomic instability. Cell 159, 1538-1548, (2014).

20 Liu, M., Duke, J. L., Richter, D. J., Vinuesa, C. G., Goodnow, C. C., Kleinstein, S. H. & Schatz, D. G. Two levels of protection for the B-cell genome during somatic hypermutation. Nature 451, 841-845, (2008).

21 Zaheen, A., Boulianne, B., Parsa, J. Y., Ramachandran, S., Gommerman, J. L. & Martin, A. AID constrains germinal center size by rendering B-cells susceptible to apoptosis. Blood 114, 547-554, (2009).

22 Nilsen, H., Stamp, G., Andersen, S., Hrivnak, G., Krokan, H. E., Lindahl, T. & Barnes, D. E. Gene-targeted mice lacking the Ung uracil-DNA glycosylase develop B-cell lymphomas. Oncogene 22, 5381-5386, (2003).

23 Andersen, S., Ericsson, M., Dai, H. Y., Pena-Diaz, J., Slupphaug, G., Nilsen, H., Aarset, H. & Krokan, H. E. Monoclonal B-cell hyperplasia and leukocyte imbalance precede development of B-cell malignancies in uracil-DNA glycosylase deficient mice. DNA Repair (Amst) 4, 1432-1441, (2005).

24 Hase, K., Takahashi, D., Ebisawa, M., Kawano, S., Itoh, K. & Ohno, H. Activationinduced cytidine deaminase deficiency causes organ-specific autoimmune disease. PloS one 3, e3033, (2008).

25 Kuraoka, M. & Kelsoe, G. A novel role for activation-induced cytidine deaminase: central B-cell tolerance. Cell Cycle 10, 3423-3424, (2011).

26 Meyers, G., Ng, Y. S., Bannock, J. M., Lavoie, A., Walter, J. E., Notarangelo, L. D., Kilic, S. S., Aksu, G., Debre, M., Rieux-Laucat, F., Conley, M. E., Cunningham-Rundles, C., Durandy, A. & Meffre, E. Activation-induced cytidine deaminase (AID) is required for B-cell tolerance in humans. Proc Nati Aced Sci USA 108, 11554-11559, (2011).

27 Okazaki, T., Okazaki, I. M., Wang, J., Sugiura, D., Nakaki, F., Yoshida, T., Kato, Y., Fagarasan, S., Muramatsu, M., Eto, T., Hioki, K. & Honjo, T. PD-1 and LAG-3 inhibitory co-receptors act synergistically to prevent autoimmunity in mice. J Exp Med 208, 395-407, (2011).

28 Zahn, A., Daugan, M., Safavi, S., Godin, D., Cheong, C., Lamarre, A. & Di Noia, J. M. Separation of function between isotype switching and affinity maturation in vivo during acute immune responses and circulating autoantibodies in UNG-deficient mice. J Immunol 190, 5949-5960, (2013).

29 Rada, C., Williams, G. T., Nilsen, H., Barnes, D. E., Lindahl, T. & Neuberger, M. S. Immunoglobulin isotype switching is inhibited and somatic hypermutation perturbed in UNG-deficient mice. Curr Biol 12, 1748-1755, (2002).

30 Nakamura, M., Kondo, S., Sugai, M., Nazarea, M., Imamura, S. & Honjo, T. High frequency class switching of an IgM+ B lymphoma clone CH12F3 to IgA+ cells. International immunology 8, 193-201, (1996).

31 Wang, Z. & Mosbaugh, D. W. Uracil-DNA glycosylase inhibitor gene of bacteriophage PBS2 encodes a binding protein specific for uracil-DNA glycosylase. J Biol Chem 264, 1163-1171, (1989).

32 Rada, C., Di Noia, J. M. & Neuberger, M. S. Mismatch recognition and uracil excision provide complementary paths to both Ig switching and the A/T-focused phase of somatic mutation. Mob Cell 16, 163-171, (2004).

33 Crabbe, L., Verdun, R. E., Haggblom, C. I. & Karlseder, J. Defective telomere lagging strand synthesis in cells lacking WRN helicase activity. Science 306, 1951-1953, (2004).

34 Verdun, R. E. & Karlseder, J. Replication and protection of telomeres. Nature 447, 924-931, (2007). 35 Bailey, S. M., Cornforth, M. N., Kurimasa, A., Chen, D. J. & Goodwin, E. H. Strandspecific Postreplicative Processing of Mammalian Telomeres. Science, 2462-2465, (2001).

36 Catalan, N., Selz, F., Imai, K., Revy, P., Fischer, A. & Durandy, A. The block in immunoglobulin class switch recombination caused by activation-induced cytidine deaminase deficiency occurs prior to the generation of DNA double strand breaks in switch mu region. J Immunol 171, 2504-2509, (2003).

37 Rush, J. S., Fugmann, S. D. & Schatz, D. G. Staggered AID-dependent DNA double strand breaks are the predominant DNA lesions targeted to S mu in Ig class switch recombination. International immunology 16, 549-557, (2004).

38 Schrader, C. E., Linehan, E. K., Mochegova, S. N., Woodland, R. T. & Stavnezer, J. Inducible DNA breaks in Ig S regions are dependent on AID and UNG. J Exp Med 202, 561-568, (2005).

39 Cortizas, E. M., Zahn, A., Najjar, M. E., Patenaude, A. M., Di Noia, J. M. & Verdun, R. E. Alternative end-joining and classical nonhomologous end-joining pathways repair different types of double-strand breaks during class-switch recombination. J Immunol 191, 5751-5763, (2013).

40 Pena-Diaz, J., Bregenhom, S., Ghodgaonkar, M., Follonier, C., Artola-Boran, M., Castor, D., Lopes, M., Sartori, A. A. & Jiricny, J. Noncanonical mismatch repair as a source of genomic instability in human cells. Mol Cell 47, 669-680, (2012).

41 Chin, L., Artandi, S. E., Shen, Q., Tam, A., Lee, S. L., Gottlieb, G. J., Greider, C. W. & DePinho, R. A. p53 deficiency rescues the adverse effects of telomere loss and cooperates with telomere dysfunction to accelerate carcinogenesis. Cell 97, 527-538, (1999).

42 d'Adda di Fagagna, F., Reaper, P. M., Clay-Farrace, L., Fiegler, H., Carr, P., Von Zglinicki, T., Saretzki, G., Carter, N. P. & Jackson, S. P. A DNA damage checkpoint response in telomere-initiated senescence. Nature 426, 194-198, (2003).

43 Herbig, U., Jobling, W. A., Chen, B. P., Chen, D. J. & Sedivy, J. M. Telomere shortening triggers senescence of human cells through a pathway involving ATM, p53, and p21(CIP1), but not p16(INK4a). Mol Cell 14, 501-513, (2004).

44 Brown, J. P., Wei, W. & Sedivy, J. M. Bypass of senescence after disruption of p21CIP1/WAF1 gene in normal diploid human fibroblasts. Science 277, 831-834, (1997).

45 Karlseder, J., Broccoli, D., Dai, Y., Hardy, S. & de Lange, T. p53- and ATM-dependent apoptosis induced by telomeres lacking TRF2. Science 283, 1321-1325, (1999).

46 Karlseder, J., Smogorzewska, A. & de Lange, T. Senescence induced by altered telomere state, not telomere loss. Science 295, 2446-2449, (2002).

47 Azzalin, C. M., Reichenbach, P., Khoriauli, L., Giulotto, E. & Lingner, J. Telomeric repeat containing RNA and RNA surveillance factors at mammalian chromosome ends. Science 318, 798-801, (2007).

48 Schoeftner, S. & Blasco, M. A. Developmentally regulated transcription of mammalian telomeres by DNA-dependent RNA polymerase II. Nat Cell Biol 10, 228-236, (2008).

49 Yu, K., Chedin, F., Hsieh, C. L., Wilson, T. E. & Lieber, M. R. R-loops at immunoglobulin class switch regions in the chromosomes of stimulated B-cells . Nature immunology 4, 442-451, (2003).

50 Pfeiffer, V., Crittin, J., Grolimund, L. & Lingner, J. The THO complex component Thp2 counteracts telomeric R-loops and telomere shortening. EMBO J 32, 2861-2871, (2013).

51 Krokan, H. E., Saetrom, P., Aas, P. A., Pettersen, H. S., Kavli, B. & Slupphaug, G. Errorfree versus mutagenic processing of genomic uracil--relevance to cancer. DNA Repair (Amst) 19, 38-47, (2014).

52 de Lange, T. How Shelterin Solves the Telomere End-Protection Problem. Cold Spring Herb Symp Quant Biol, (2011).

53 Petersen, S., Casellas, R., Reina-San-Martin, B., Chen, H. T., Difilippantonio, M. J., Wilson, P. C., Hanitsch, L., Celeste, A., Muramatsu, M., Pilch, D. R., Redon, C., Ried, T., Bonner, W. M., Honjo, T., Nussenzweig, M. C. & Nussenzweig, A. AID is required to initiate Nbs1/gamma-H2AX focus formation and mutations at sites of class switching. Nature 414, 660-665, (2001).

54 Sharbeen, G., Yee, C. W., Smith, A. L. & Jolly, C. J. Ectopic restriction of DNA repair reveals that UNG2 excises AID-induced uracils predominantly or exclusively during G1 phase. J Exp Med 209, 965-974, (2012).

55 de Miranda, N. F., Peng, R., Georgiou, K., Wu, C., Falk Sorqvist, E., Berglund, M., Chen, L., Gao, Z., Lagerstedt, K., Lisboa, S., Roos, F., van Wezel, T., Teixeira, M. R., Rosenquist, R., Sundstrom, C., Enblad, G., Nilsson, M., Zeng, Y., Kipling, D. & Pan- Hammarstrom, Q. DNA repair genes are selectively mutated in diffuse large B-cell lymphomas. J Exp Med 210, 1729-1742, (2013).

56 Couronne, L., Ruminy, P., Waultier-Rascalou, A., Rainville, V., Comic, M., Picquenot, J. M., Figeac, M., Bastard, C., Tilly, H. & Jardin, F. Mutation mismatch repair gene deletions in diffuse large B-cell lymphoma. Leuk Lymphoma 54, 1079-1086, (2013).

57 Imai, K., Slupphaug, G., Lee, W. I., Revy, P., Nonoyama, S., Catalan, N., Yel, L., Forveille, M., Kavli, B., Krokan, H. E., Ochs, H. D., Fischer, A. & Durandy, A. Human uracil-DNA glycosylase deficiency associated with profoundly impaired immunoglobulin class-switch recombination. Nature immunology 4, 1023-1028, (2003).

58 Nilsen, H., An, Q. & Lindahl, T. Mutation frequencies and AID activation state in B-cell lymphomas from Ung-deficient mice. Oncogene 24, 3063-3066, (2005).

59 Muramatsu, M., Kinoshita, K., Fagarasan, S., Yamada, S., Shinkai, Y. & Honjo, T. Class switch recombination and hypermutation require activation-induced cytidine deaminase (AID), a potential RNA editing enzyme. Cell 102, 553-563, (2000).

60 Nilsen, H., Steinsbekk, K. S., Otterlei, M., Slupphaug, G., Aas, P. A. & Krokan, H. E. Analysis of uracil-DNA glycosylases from the murine Ung gene reveals differential expression in tissues and in embryonic development and a subcellular sorting pattern that differs from the human homologues. Nucleic Acids Res 28, 2277-2285, (2000).

61 Verdun, R. E., Crabbe, L., Haggblom, C. & Karlseder, J. Functional human telomeres are recognized as DNA damage in G2 of the cell cycle. Mol Cell 20, 551-561, (2005).

62 Albesiano, E. et al. Activation-induced cytidine deaminase in chronic lymphocytic leukemia B cells: expression as multiple forms in a dynamic, variably sized fraction of the clone. Blood 102, 3333-3339, doi:10.1182/blood-2003-05-1585 (2003).

63 Durandy, A., Peron, S., Taubenheim, N. & Fischer, A. Activation-induced cytidine deaminase: structure-function relationship as based on the study of mutants. Hum Mutat 27, 1185-1191 (2006).

64 Krosky et al., Mimicking damaged DNA with a small molecule inhibitor of human UNG2 Nucl. Acids Res. (2006) 34 (20): 5872-5879. doi: 10.1093/nar/gkl747 First published online: Oct. 24, 2006.

65 Acharya et al. 2003, Microbiology. 2003 Jul;149(Pt 7):1647-58; Mol et al, 1995, Cell, Vol 82, p 701-708.

66 Methot SP, et al. “Consecutive interactions with HSP90 and eEF1A underlie a functional maturation and storage pathway of AID in the cytoplasm” J Exp Med. 2015 Apr. 6; 212(4):581-96. doi: 10.1084/jem.20141157. Epub 2015 Mar. 30.

67 Zhang M, Xiang S, Joo HY, Wang L, Williams KA, Liu W, Hu C, Tong D, Haakenson J, Wang C, Zhang S, Pavlovicz RE, Jones A, Schmidt KH, Tang J, Dong H, Shan B, Fang B, Radhakrishnan R, Glazer PM, Matthias P, Koomen J, Seto E, Bepler G, Nicosia SV, Chen J, Li C, Gu L, Li GM, Bai W, Wang H, Zhang X. HDAC6 deacetylates and ubiquitinates MSH2 to maintain proper levels of MutSa. Mol Cell. 2014 Jul. 3;55(1):31-46. doi: 10.10161j.molce1.2014.04.028. Epub 2014 May 29. PubMed PMID: 24882211; PubMed Central PMCID: PMC4188514.

68 Matsumoto, Y. et aL Helicobacter pylori infection triggers aberrant expression of activation-induced cytidine deaminase in gastric epithelium. Nat Med 13, 470-476 (2007).

69 Di Noia J1, Neuberger MS Altering the pathway of immunoglobulin hypermutation by inhibiting uracil-DNA glycosylase. Nature. 2002 Sep. 5;419 (6902):43-8. Epub 2002 Jul. 31.

70 Zahn, A., Eranki, A.K., Patenaude, A.M., Methot, S.P., Fifield, H., Cortizas, E.M., Foster, P., Imai, K., Durandy, A., Larijani, M., et al. (2014). Activation induced deaminase C-terminal domain links DNA breaks to end protection and repair during class switch recombination. Proc Natl Acad Sci U S A.71 Verdun, R.E., Crabbe, L., Haggblom, C., and Karlseder, J. (2005). Functional human telomeres are recognized as DNA damage in G2 of the cell cycle. Mol Cell 20, 551-561.

1. A method for the prevention and/or treatment of an activation-induced deaminase (AID)-associated disease in a subject in need thereof, said method comprising administering an effective amount of an uracil-DNA glycosylase (UNG) inhibitor, or a composition comprising the inhibitor, and a pharmaceutically acceptable carrier, to a subject having pathogenic cells expressing AID, uracil-DNA glycosylase (UNG) and mismatch repair pathway (MMR).

2. The method of claim 1, wherein the AID-associated disease is an AID-associated neoplastic disease and the pathogenic cells are neoplastic cells.

3. The method of claim 2, wherein the AID-associated neoplastic disease is a B-cell lymphoma or leukemia.

4. The method of claim 1, further comprising detecting (i) AID expression and/or activity; (ii) UNG expression and/or activity; (iii) MMR expression and/or activity; or (iv) a combination of at least two of (i) to (iii) in the pathogenic cells.

5. The method of claim 1, further comprising detecting (i) AID expression and/or activity; (ii) UNG expression and/or activity; and (iii) MMR expression and/or activity in the pathogenic cells.

6. The method of claim 1, wherein the UNG inhibitor is an Ugi peptide.

7. The method of claim 1, further comprising administering at least one further therapeutic agent to the subject.

8. The method of claim 7, wherein the at least one further therapeutic agent comprises at least one compound that favors AID nuclear localization.

9. The method of claim 8, wherein the compound that favors AID nuclear localization is an eukaryotic elongation factor 1 α (eEF1A) inhibitor.

10. The method of claim 9, wherein the eEF1A inhibitor is didemnin B (DidB) or cytotrienin A (CytA)).

11. A method for stratifying a subject having an activation-induced deaminase (AID)-associated disease comprising:
(i) detecting AID expression and/or activity;
(ii) detecting uracil-DNA glycosylase (UNG) expression and/or activity;
(iii) detecting mismatch repair pathway (MMR) expression and/or activity; or
(iv) detecting a combination of at least two of (i) to (iii), in the pathogenic cells, wherein said detecting enables the stratification of the subject.

12. The method of claim 11, further comprising administering an effective amount of an uracil-DNA glycosylase (UNG) inhibitor to the subject.

13. The method of claim 11, further comprising administering at least one further therapeutic agent to the subject.

14. The method of claim 13, wherein the at least one further therapeutic agent comprises at least one compound that favors AID nuclear localization.

15. The method of claim 14, wherein the compound that favors AID nuclear localization is an eukaryotic elongation factor 1 α (eEF1A) inhibitor.

16. The method of claim 15, wherein the eEF1A inhibitor is didemnin B (DidB) or cytotrienin A (CytA)).

17-38. (canceled)

39. A kit for preventing and/or treating an activation-induced deaminase (AID)-associated disease in a subject, comprising an uracil-DNA glycosylase (UNG) inhibitor or a composition comprising the inhibitor, and a pharmaceutically acceptable carrier, and at least one further therapeutic agent.

40. The kit of claim 39, wherein the AID-associated disease is an AID-associated neoplastic disease.

41. The kit of claim 39, wherein the UNG inhibitor is an Ugi peptide.

42. The kit of claim 39, wherein the at least one further therapeutic agent comprises at least one compound that favors AID nuclear localization, preferably the compound that favors AID nuclear localization is an eukaryotic elongation factor 1 α (eEF1A) inhibitor, more preferably the eEF1A inhibitor is didemnin B (DidB) or cytotrienin A (CytA)).

43-44. (canceled)
